SEQUENCE LISTING (84 sequences)

SEQ ID NO: 1 - protein, 217 aa, synthetic construct
  REGION 1..217: note = Synthetic Polypeptide
APEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGK

SEQ ID NO: 2 - protein, 217 aa, synthetic construct
  REGION 1..217: note = Synthetic Polypeptide
APEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGK

SEQ ID NO: 3 - protein, 217 aa, synthetic construct
  REGION 1..217: note = Synthetic Polypeptide
APEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 4 - protein, 11 aa, synthetic construct
  REGION 1..11: note = Synthetic Polypeptide
SKYGPPCPSCP

SEQ ID NO: 5 - protein, 11 aa, synthetic construct
  REGION 1..11: note = Synthetic Polypeptide
SKYGPPCPPCP

SEQ ID NO: 6 - protein, 6 aa, synthetic construct
  REGION 1..6: note = Synthetic Polypeptide
SKYGPP

SEQ ID NO: 7 - protein, 6 aa, synthetic construct
  REGION 1..6: note = Synthetic Polypeptide
IEGRMD

SEQ ID NO: 8 - protein, 9 aa, synthetic construct
  REGION 1..9: note = Synthetic Polypeptide
GGGVPRDCG

SEQ ID NO: 9 - protein, 15 aa, synthetic construct
  REGION 1..15: note = Synthetic Polypeptide
IEGRMDGGGGAGGGG

SEQ ID NO: 10 - protein, 8 aa, synthetic construct
  REGION 1..8: note = Synthetic Polypeptide
GGGSGGGS

SEQ ID NO: 11 - protein, 12 aa, synthetic construct
  REGION 1..12: note = Synthetic Polypeptide
GGGSGGGGSGGG

SEQ ID NO: 12 - protein, 14 aa, synthetic construct
  REGION 1..14: note = Synthetic Polypeptide
EGKSSGSGSESKST

SEQ ID NO: 13 - protein, 4 aa, synthetic construct
  REGION 1..4: note = Synthetic Polypeptide
GGSG

SEQ ID NO: 14 - protein, 12 aa, synthetic construct
  REGION 1..12: note = Synthetic Polypeptide
GGSGGGSGGGSG

SEQ ID NO: 15 - protein, 15 aa, synthetic construct
  REGION 1..15: note = Synthetic Polypeptide
EAAAKEAAAKEAAAK

SEQ ID NO: 16 - protein, 20 aa, synthetic construct
  REGION 1..20: note = Synthetic Polypeptide
EAAAREAAAREAAAREAAAR

SEQ ID NO: 17 - protein, 17 aa, synthetic construct
  REGION 1..17: note = Synthetic Polypeptide
GGGGSGGGGSGGGGSAS

SEQ ID NO: 18 - protein, 9 aa, synthetic construct
  REGION 1..9: note = Synthetic Polypeptide
GGGGAGGGG

SEQ ID NO: 19 - skipped

SEQ ID NO: 20 - protein, 6 aa, synthetic construct
  REGION 1..6: note = Synthetic Polypeptide
GSGSGS

SEQ ID NO: 21 - protein, 10 aa, synthetic construct
  REGION 1..10: note = Synthetic Polypeptide
GSGSGSGSGS

SEQ ID NO: 22 - protein, 7 aa, synthetic construct
  REGION 1..7: note = Synthetic Polypeptide
GGGGSAS

SEQ ID NO: 23 - protein, 20 aa, synthetic construct
  REGION 1..20: note = Synthetic Polypeptide
APAPAPAPAPAPAPAPAPAP

SEQ ID NO: 24 - protein, 4 aa, synthetic construct
  REGION 1..4: note = Synthetic Polypeptide
CPPC

SEQ ID NO: 25 - protein, 5 aa, synthetic construct
  REGION 1..5: note = Synthetic Polypeptide
GGGGS

SEQ ID NO: 26 - protein, 10 aa, synthetic construct
  REGION 1..10: note = Synthetic Polypeptide
GGGGSGGGGS

SEQ ID NO: 27 - protein, 15 aa, synthetic construct
  REGION 1..15: note = Synthetic Polypeptide
GGGGSGGGGSGGGGS

SEQ ID NO: 28 - protein, 20 aa, synthetic construct
  REGION 1..20: note = Synthetic Polypeptide
GGGGSGGGGSGGGGSGGGGS

SEQ ID NO: 29 - protein, 25 aa, synthetic construct
  REGION 1..25: note = Synthetic Polypeptide
GGGGSGGGGSGGGGSGGGGSGGGGS

SEQ ID NO: 30 - protein, 30 aa, synthetic construct
  REGION 1..30: note = Synthetic Polypeptide
GGGGSGGGGSGGGGSGGGGSGGGGSGGGGS

SEQ ID NO: 31 - protein, 35 aa, synthetic construct
  REGION 1..35: note = Synthetic Polypeptide
GGGGSGGGGSGGGGSGGGGSGGGGSGGGGSGGGGS

SEQ ID NO: 32 - protein, 40 aa, synthetic construct
  REGION 1..40: note = Synthetic Polypeptide
GGGGSGGGGSGGGGSGGGGSGGGGSGGGGSGGGGSGGGGS

SEQ ID NO: 33 - protein, 16 aa, synthetic construct
  REGION 1..16: note = Synthetic Polypeptide
GGSGGSGGGGSGGGGS

SEQ ID NO: 34 - protein, 8 aa, synthetic construct
  REGION 1..8: note = Synthetic Polypeptide
GGGGGGGG

SEQ ID NO: 35 - protein, 6 aa, synthetic construct
  REGION 1..6: note = Synthetic Polypeptide
GGGGGG

SEQ ID NO: 36 - protein, 5 aa, synthetic construct
  REGION 1..5: note = Synthetic Polypeptide
EAAAK

SEQ ID NO: 37 - protein, 10 aa, synthetic construct
  REGION 1..10: note = Synthetic Polypeptide
EAAAKEAAAK

SEQ ID NO: 38 - protein, 15 aa, synthetic construct
  REGION 1..15: note = Synthetic Polypeptide
EAAAKEAAAKEAAAK

SEQ ID NO: 39 - protein, 12 aa, synthetic construct
  REGION 1..12: note = Synthetic Polypeptide
AEAAAKEAAAKA

SEQ ID NO: 40 - protein, 17 aa, synthetic construct
  REGION 1..17: note = Synthetic Polypeptide
AEAAAKEAAAKEAAAKA

SEQ ID NO: 41 - protein, 22 aa, synthetic construct
  REGION 1..22: note = Synthetic Polypeptide
AEAAAKEAAAKEAAAKEAAAKA

SEQ ID NO: 42 - protein, 27 aa, synthetic construct
  REGION 1..27: note = Synthetic Polypeptide
AEAAAKEAAAKEAAAKEAAAKEAAAKA

SEQ ID NO: 43 - protein, 46 aa, synthetic construct
  REGION 1..46: note = Synthetic Polypeptide
AEAAAKEAAAKEAAAKEAAAKALEAEAAAKEAAAKEAAAKEAAAKA

SEQ ID NO: 44 - protein, 5 aa, synthetic construct
  REGION 1..5: note = Synthetic Polypeptide
PAPAP

SEQ ID NO: 45 - protein, 18 aa, synthetic construct
  REGION 1..18: note = Synthetic Polypeptide
KESGSVSSEQLAQFRSLD

SEQ ID NO: 46 - protein, 12 aa, synthetic construct
  REGION 1..12: note = Synthetic Polypeptide
GSAGSAAGSGEF

SEQ ID NO: 47 - protein, 5 aa, synthetic construct
  REGION 1..5: note = Synthetic Polypeptide
GGGSE

SEQ ID NO: 48 - protein, 5 aa, synthetic construct
  REGION 1..5: note = Synthetic Polypeptide
GSESG

SEQ ID NO: 49 - protein, 5 aa, synthetic construct
  REGION 1..5: note = Synthetic Polypeptide
GSEGS

SEQ ID NO: 50 - protein, 35 aa, synthetic construct
  REGION 1..35: note = Synthetic Polypeptide
GEGGSGEGSSGEGSSSEGGGSEGGGSEGGGSEGGS

SEQ ID NO: 51 - protein, 234 aa, synthetic construct
  REGION 1..234: note = Synthetic Polypeptide
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 52 - protein, 234 aa, synthetic construct
  REGION 1..234: note = Synthetic Polypeptide
SKYGPPCPSCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 53 - protein, 234 aa, synthetic construct
  REGION 1..234: note = Synthetic Polypeptide
SKYGPPCPSCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 54 - protein, 234 aa, synthetic construct
  REGION 1..234: note = Synthetic Polypeptide
SKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 55 - protein, 234 aa, synthetic construct
  REGION 1..234: note = Synthetic Polypeptide
SKYGPPCPPCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 56 - protein, 234 aa, synthetic construct
  REGION 1..234: note = Synthetic Polypeptide
SKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 57 - protein, 235 aa, synthetic construct
  REGION 1..235: note = Synthetic polypeptide
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 58 - protein, 182 aa, synthetic construct
  REGION 1..182: note = Synthetic polypeptide
TMAIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 59 - protein, 131 aa, synthetic construct
  REGION 1..131: note = Synthetic polypeptide
SSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 60 - protein, 264 aa, synthetic construct
  REGION 1..264: note = Synthetic polypeptide
PRFMGTISDVKTNVQLLKGRVDNISTLDSEIKKNSDGMEAAGVQIQMVNESLGYVRSQFLKLKTSVEKANAQIQILTRSWEEVSTLNAQIPELKSDLEKASALNTKIRALQGSLENMSKLLKRQNDILQVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEP

SEQ ID NO: 61 - protein, 136 aa, synthetic construct
  REGION 1..136: note = Synthetic polypeptide
QVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEP

SEQ ID NO: 62 - protein, 346 aa, synthetic construct
  REGION 1..346: note = Synthetic polypeptide
QVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 63 - protein, 125 aa, synthetic construct
  REGION 1..125: note = Synthetic polypeptide
HPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICK

SEQ ID NO: 64 - protein, 168 aa, synthetic construct
  REGION 1..168: note = Synthetic polypeptide
TYHFTYGETGKRLSELHSYHSSLTCFSEGTKVPAWGCCPASWKSFGSSCYFISSEEKVWSKSEQNCVEMGAHLVVFNTEAEQNFIVQQLNESFSYFLGLSDPQGNNNWQWIDKTPYEKNVRFWHLGEPNHSAEQCASIVFWKPTGWGWNDVICETRRNSICEMNKIYL

SEQ ID NO: 65 - protein, 118 aa, synthetic construct
  REGION 1..118: note = Synthetic polypeptide
FGSSCYFISSEEKVWSKSEQNCVEMGAHLVVFNTEAEQNFIVQQLNESFSYFLGLSDPQGNNNWQWIDKTPYEKNVRFWHLGEPNHSAEQCASIVFWKPTGWGWNDVICETRRNSICE

SEQ ID NO: 66 - protein, 655 aa, synthetic construct
  REGION 1..655: note = Synthetic polypeptide
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDTMAIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 67 - protein, 604 aa, synthetic construct
  REGION 1..604: note = Synthetic polypeptide
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 68 - protein, 737 aa, synthetic construct
  REGION 1..737: note = Synthetic polypeptide
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDPRFMGTISDVKTNVQLLKGRVDNISTLDSEIKKNSDGMEAAGVQIQMVNESLGYVRSQFLKLKTSVEKANAQIQILTRSWEEVSTLNAQIPELKSDLEKASALNTKIRALQGSLENMSKLLKRQNDILQVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEP

SEQ ID NO: 69 - protein, 609 aa, synthetic construct
  REGION 1..609: note = Synthetic polypeptide
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDQVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEP

SEQ ID NO: 70 - protein, 819 aa, synthetic construct
  REGION 1..819: note = Synthetic polypeptide
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 71 - protein, 598 aa, synthetic construct
  REGION 1..598: note = Synthetic polypeptide
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICK

SEQ ID NO: 72 - protein, 641 aa, synthetic construct
  REGION 1..641: note = Synthetic polypeptide
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDTYHFTYGETGKRLSELHSYHSSLTCFSEGTKVPAWGCCPASWKSFGSSCYFISSEEKVWSKSEQNCVEMGAHLVVFNTEAEQNFIVQQLNESFSYFLGLSDPQGNNNWQWIDKTPYEKNVRFWHLGEPNHSAEQCASIVFWKPTGWGWNDVICETRRNSICEMNKIYL

SEQ ID NO: 73 - protein, 232 aa, synthetic construct
  REGION 1..232: note = Synthetic polypeptide
EPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 684 aa, synthetic construct
  REGION 1..684: note = Synthetic polypeptide
PRFMGTISDVKTNVQLLKGRVDNISTLDSEIKKNSDGMEAAGVQIQMVNESLGYVRSQFLKLKTSVEKANAQIQILTRSWEEVSTLNAQIPELKSDLEKASALNTKIRALQGSLENMSKLLKRQNDILQVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEPEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDTMAIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 75 - protein, 505 aa, synthetic construct
  REGION 1..505: note = Synthetic polypeptide
QVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEPEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 76 - protein, 848 aa, synthetic construct
  REGION 1..848: note = Synthetic polypeptide
PRFMGTISDVKTNVQLLKGRVDNISTLDSEIKKNSDGMEAAGVQIQMVNESLGYVRSQFLKLKTSVEKANAQIQILTRSWEEVSTLNAQIPELKSDLEKASALNTKIRALQGSLENMSKLLKRQNDILQVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEPEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 77 - protein, 499 aa, synthetic construct
  REGION 1..499: note = Synthetic polypeptide
QVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEPEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICK

SEQ ID NO: 78 - protein, 670 aa, synthetic construct
  REGION 1..670: note = Synthetic polypeptide
PRFMGTISDVKTNVQLLKGRVDNISTLDSEIKKNSDGMEAAGVQIQMVNESLGYVRSQFLKLKTSVEKANAQIQILTRSWEEVSTLNAQIPELKSDLEKASALNTKIRALQGSLENMSKLLKRQNDILQVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEPEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDTYHFTYGETGKRLSELHSYHSSLTCFSEGTKVPAWGCCPASWKSFGSSCYFISSEEKVWSKSEQNCVEMGAHLVVFNTEAEQNFIVQQLNESFSYFLGLSDPQGNNNWQWIDKTPYEKNVRFWHLGEPNHSAEQCASIVFWKPTGWGWNDVICETRRNSICEMNKIYL

SEQ ID NO: 79 - protein, 542 aa, synthetic construct
  REGION 1..542: note = Synthetic polypeptide
QVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEPEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDTYHFTYGETGKRLSELHSYHSSLTCFSEGTKVPAWGCCPASWKSFGSSCYFISSEEKVWSKSEQNCVEMGAHLVVFNTEAEQNFIVQQLNESFSYFLGLSDPQGNNNWQWIDKTPYEKNVRFWHLGEPNHSAEQCASIVFWKPTGWGWNDVICETRRNSICEMNKIYL

SEQ ID NO: 80 - protein, 235 aa, synthetic construct; identical to SEQ ID NO: 57

SEQ ID NO: 81 - protein, 643 aa, Synthetic construct
VPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKIEGRMDGHNSGRNPEEKDNFLSRNKENHKPTESSLDEKVAPSKASQTTGGFSQPCLPNWIMHGKSCYLFSFSGNSWYGSKRHCSQLGAHLLKIDNSKEFEFIESQTSSHRINAFWIGLSRNQSEGPWFWEDGSAFFPNSFQVRNTAPQESLLHNCVWIHGSEVYNQICNTSSYSICEKEL

SEQ ID NO: 82 - protein, 635 aa, Synthetic construct
VPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKIEGRMDVKVYKIPSSQEENNQMNVYQELTQLKAGVDRLCRSCPWDWTHFQGSCYFFSVAQKSWNDSATACHNVGAQLVVIKSDEEQNFLQQTSKKRGYTWMGLIDMSKESTWYWVDGSPLTLSFMKYWSKGEPNNLGEEDCAEFRDDGWNDTKCTNKKFWICKKLSTSCPSK

SEQ ID NO: 83 - protein, 635 aa, Synthetic construct
VPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKIEGRMDIMDQPSRRLYELHTYHSSLTCFSEGTMVSEKMWGCCPNHWKSFGSSCYLISTKENFWSTSEQNCVQMGAHLVVINTEAEQNFITQQLNESLSYFLGLSDPQGNGKWQWIDDTPFSQNVRFWHPHEPNLPEERCVSIVYWNPSKWGWNDVFCDSKHNSICEMKKIYL

SEQ ID NO: 84 - protein, 738 aa, Synthetic construct
VPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKIEGRMDQAVFYPRLMGKILDVKSDAQMLKGRVDNISTLGSDLKTERGRVDDAEVQMQIVNTTLKRVRSQILSLETSMKIANDQLQILTMSWGEVDSLSAKIPELKRDLDKASALNTKVQGLQNSLENVNKLLKQQSDILEMVARGWKYFSGNFYYFSRTPKTWYSAEQFCISRKAHLTSVSSESEQKFLYKAADGIPHWIGLTKAGSEGDWYWVDQTSFNKEQSRRFWIPGEPNNAGNNEHCANIRVSALKCWNDGPCDNTFLFICKRPYVQTTE

(19) United States
(12) Patent Application Publication
(10) Pub. No.: US 2026/0139030 A1
(43) Pub. Date: May 21, 2026

(54) CHIMERIC PROTEINS FOR TREATING CUTANEOUS INFLAMMATION

(71) Applicant: Shattuck Labs, Inc., Austin, TX (US)

(72) Inventors: Taylor SCHREIBER, Austin, TX (US); George FROMM, Austin, TX (US); Casey SHUPTRINE, Austin, TX (US)

(21) Appl. No.: 18/705,810

(22) PCT Filed: Nov. 1, 2022
(86) PCT No.: PCT/US2022/079046

§ 371 Date: Apr. 29, 2024

Related U.S. Application Data
(60) Provisional application No. 63/274,232, filed on Nov. 1, 2021.
(60) Provisional application No. 63/320,628, filed on Mar. 16, 2022.
(60) Provisional application No. 63/325,568, filed on Mar. 30, 2022.
(60) Provisional application No. 63/369,836, filed on Jul. 29, 2022.

(51) Int. Cl. C07K 14/705; A61K 31/192; A61K 31/196; A61K 31/235; A61K 31/405; A61K 31/407; A61K 31/421; A61K 31/573; A61K 31/58; A61K 31/60; A61K 31/616; A61K 31/618; A61K 31/635; A61K 38/00; A61K 38/17; A61K 45/06; C07K 16/24

(52) U.S. Cl. CPC C07K 14/70578; A61K 31/192; A61K 31/196; A61K 31/235; A61K 31/405; A61K 31/407; A61K 31/421; A61K 31/573; A61K 31/58; A61K 31/60; A61K 31/616; A61K 31/618; A61K 31/635; A61K 38/177; A61K 38/178; A61K 45/06; C07K 14/7056; C07K 16/244; A61K 38/00; C07K 2319/30

(57) ABSTRACT

The present disclosure relates to, inter alia, compositions and methods, including heterologous chimeric proteins that find use, inter alia, in the treatment of inflammatory conditions of the integumentary system.

### PRIORITY

This application claims the benefit of, and priority to, U.S. Provisional Application No. 63/274,232, filed Nov. 1, 2021, U.S. Provisional Application No. 63/320,628, filed Mar. 16, 2022, U.S. Provisional Application No. 63/325,568, filed Mar. 30, 2022, and U.S. Provisional Application No. 63/369,836, filed Jul. 29, 2022, the contents of each of which are hereby incorporated by reference in their entirety.

### TECHNICAL FIELD

The present disclosure relates to, inter alia, compositions and methods, including heterologous chimeric proteins, or nucleic acids encoding the chimeric proteins, that find use, inter alia, in the treatment of inflammatory conditions of the integumentary system.

### SEQUENCE LISTING

The instant application contains a sequence listing, which has been submitted in XML format via Patent Center. The contents of the XML copy named “SHK-054PC_116981-5054_Sequence Listing”, which was created on Oct. 31, 2022 and is 117,810 bytes in size, are incorporated herein by reference in their entirety.

### BACKGROUND

Psoriasis is an inflammatory skin disorder characterized by red, itchy and scaly patches on skin. Psoriasis is believed to affect 2-3% people worldwide and be caused by immune pathways. Traditionally, topical corticosteroids (e.g., triamcinolone acetonide and clobetasol propionate), topical keratolytics (e.g., salicylic acid), topical vitamin D3 analogs (e.g., calcitriol), oral or topical retinoids (e.g., tazarotene and acitretin), systemic cytotoxic agents (e.g., methotrexate), and immunosuppressive drugs (e.g., cyclosporin) are used to treat psoriasis. Recently, targeted agents have been developed. These agents include the TNFα antagonists such as anti-TNFα antibodies (e.g., infliximab and adalimumab) and etanercept, a fusion protein of TNF receptor TNFR2 and an Fc domain; anti-IL-12B (anti-IL-12 and anti-IL-23) antibodies (e.g., ustekinumab); anti-IL-23 antibodies (e.g., guselkumab); and anti-IL-17 antibodies (e.g., secukinumab).

There is no cure for psoriasis because all aforementioned treatments provide temporary relief of symptoms rather than the treatment of the underlying disease. As a consequence, these treatments at best cause remission of symptoms, but relapse of usually follows. In exchange for the temporary relief of symptoms, these treatments can cause mild to severe side effects such as skin irritation to susceptibility to serious infections, including tuberculosis. the aforementioned treatments also provide temporary relief of symptoms rather than the treatment of the underlying disease for psoriatic arthritis (PsA), plaque psoriasis, rheumatoid arthritis (RA), juvenile arthritis, ankylosing spondylitis, inflammatory bowel disease (IBD), ulcerative colitis (UC), Crohn's disease. Accordingly, new therapies and therapeutic approaches are required to treat psoriasis and other inflammatory conditions, including inflammatory skin conditions and inflammatory GI tract conditions.

### SUMMARY

Accordingly, in various aspects, the present disclosure provides compositions and methods that are useful, inter alia, in the treatment of inflammatory conditions of the integumentary system, e.g., cutaneous inflammation of the integumentary system, including, for instance, psoriasis.

In one aspect, the present disclosure provides a chimeric protein having a general structure of: N terminus-(a)-(b)-(c)-C terminus, or a nucleic acid encoding the chimeric protein, wherein: (a) is a first domain comprising a portion of TNF receptor (TNFR2) that is capable of binding TNFa and/or capable of oligomerizing with a cellular TNF receptor; (b) is a linker adjoining the first domain and a second domain, optionally comprising a hinge-CH2-CH3 Fc domain, and (c) is a second domain comprising a portion of a C-type lectin receptor (CLR) capable of binding a ligand.

In embodiments, the portion of TNFR2 comprises the extracellular domain of TNFR2, or a fragment thereof. In embodiments, the portion of TNFR2 comprises an amino acid sequence that is at least about 90%, or at least about 95%, or at least about 97%, or at least about 98%, or at least about 99% identical to the amino acid sequence of SEQ ID NO: 57.

In embodiments, the CLR is selected from C-Type Lectin Domain Containing 7A(Clec7A), langerin, Dendritic Cell-Specific Intercellular adhesion molecule-3-Grabbing Non-integrin (DC-SIGN), and dendritic cell-associated C-type lectin-2 (Dectin-2).

In embodiments, the second domain comprises a portion of Clec7a. In embodiments, the portion of Clec7a comprises the extracellular domain of Clec7a, or a fragment thereof capable of binding a beta-1,3-linked and/or beta-1,6-linked glucan. In embodiments, the portion of Clec7a comprises an amino acid sequence that is at least about 90%, or at least about 95%, or at least about 97%, or at least about 98%, or at least about 99% identical to the amino acid sequence of SEQ ID NO: 58 or SEQ ID NO: 59. In embodiments, the chimeric protein comprises: an extracellular domain of TNFR2 comprising an amino acid sequence that is at least about 90%, or at least about 95%, or at least about 97%, or at least about 98%, or at least about 99% identical to the amino acid sequence of SEQ ID NO: 57; a portion of Clec7a comprising an amino acid sequence that is at least about 90%, or at least about 95%, or at least about 97%, or at least about 98%, or at least about 99% identical to the amino acid sequence of SEQ ID NO: 58 or SEQ ID NO: 59; and a linker adjoining the extracellular domains.

In embodiments, the second domain comprises a portion of langerin. In embodiments, the portion of langerin comprises the extracellular domain of langerin, or a fragment thereof capable of binding a sulfated glycan, a mannosylated glycan, a keratan sulfate (KS) and/or a beta-glucan. In embodiments, the portion of langerin comprises an amino acid sequence that is at least about 90%, or at least about 95%, or at least about 97%, or at least about 98%, or at least about 99% identical to the amino acid sequence of SEQ ID NO: 60 or SEQ ID NO: 61. In embodiments, the chimeric protein comprises: an extracellular domain of TNFR2 comprising an amino acid sequence that is at least about 90%, or at least about 95%, or at least about 97%, or at least about 98%, or at least about 99% identical to the amino acid sequence of SEQ ID NO: 57; a portion of langerin comprising an amino acid sequence that is at least about 90%, or at least about 95%, or at least about 97%, or at least about 98%, or at least about 99% identical to the amino acid sequence of SEQ ID NO: 60 or SEQ ID NO: 61; and a linker adjoining the extracellular domains.

In embodiments, the second domain comprises a portion of DC-SIGN. In embodiments, the portion of DC-SIGN comprises the extracellular domain of DC-SIGN, or a fragment thereof capable of binding Intercellular Adhesion Molecule 2 (ICAM2) and/or Intercellular Adhesion Molecule 3 (ICAM3). In embodiments, the portion of DC-SIGN comprises an amino acid sequence that is at least about 90%, or at least about 95%, or at least about 97%, or at least about 98%, or at least about 99% identical to the amino acid sequence of SEQ ID NO: 62 or SEQ ID NO: 63. In embodiments, the chimeric protein comprises: an extracellular domain of TNFR2 comprising an amino acid sequence that is at least about 90%, or at least about 95%, or at least about 97%, or at least about 98%, or at least about 99% identical to the amino acid sequence of SEQ ID NO: 57; a portion of DC-SIGN comprising an amino acid sequence that is at least about 90%, or at least about 95%, or at least about 97%, or at least about 98%, or at least about 99% identical to the amino acid sequence of SEQ ID NO: 62 or SEQ ID NO: 63; and a linker adjoining the extracellular domains.

In embodiments, the second domain comprises a portion of Dectin-2. In embodiments, the portion of Dectin-2 comprises the extracellular domain of Dectin-2, or a fragment thereof capable of binding an alpha-mannan. In embodiments, the portion of Dectin-2 comprises an amino acid sequence that is at least about 90%, or at least about 95%, or at least about 97%, or at least about 98%, or at least about 99% identical to the amino acid sequence of SEQ ID NO: 64 or SEQ ID NO: 65. In embodiments, the chimeric protein comprises: an extracellular domain of TNFR2 comprising an amino acid sequence that is at least about 90%, or at least about 95%, or at least about 97%, or at least about 98%, or at least about 99% identical to the amino acid sequence of SEQ ID NO: 57; a portion of Dectin-2 comprising an amino acid sequence that is at least about 90%, or at least about 95%, or at least about 97%, or at least about 98%, or at least about 99% identical to the amino acid sequence of SEQ ID NO: 64 or SEQ ID NO: 65; and a linker adjoining the extracellular domains.

In embodiments, the hinge-CH2-CH3 Fc domain is derived from IgG1 or IgG4, e.g., human IgG1 or IgG4. In embodiments, the hinge-CH2-CH3 Fc domain comprises an amino acid sequence that is at least about 95% identical to the amino acid sequence of SEQ ID NO: 1, SEQ ID NO: 2, SEQ ID NO: 3, or SEQ ID NO: 73. In embodiments, the linker further comprises the linker comprises one or more joining linkers, such joining linkers independently selected from SEQ ID NOs: 4 to 50. In embodiments, the linker comprises two or more joining linkers each joining linker independently selected from SEQ ID NOs: 4 to 50; wherein one joining linker is N terminal to the hinge-CH2-CH3-Fc domain and another joining linker is C terminal to the hinge-CH2-CH3-Fc domain.

In one aspect, the present disclosure provides an isolated polynucleotide encoding the chimeric protein of any of the embodiments disclosed herein. In embodiments, the polynucleotide is or comprises an mRNA such as a modified mRNA (mmRNA). In embodiments, the polynucleotide is or comprises an mmRNA. In embodiments, the mmRNA comprises one or more nucleoside modifications. In embodiments, the mmRNA further comprises a 5′-cap and/or a poly A tail.

In one aspect, the present disclosure provides a pharmaceutical composition comprising a pharmaceutically acceptable excipient or carrier, and the chimeric protein of any of the embodiments disclosed herein, the isolated polynucleotide of any of the embodiments disclosed herein, the mmRNA of any of the embodiments disclosed herein, the vector of any of the embodiments disclosed herein, or the host cell of any of the embodiments disclosed herein. In embodiments, the pharmaceutical composition comprises the mmRNA of any of the embodiments disclosed herein.

In one aspect, the present disclosure provides a method of treating or preventing inflammation of the integumentary system, the method comprising administering to a subject the nucleic acid, e.g., the mmRNA of any of the embodiments disclosed herein.

In one aspect, the present disclosure provides a method of treating or preventing inflammation of the integumentary system, the method comprising administering to a subject the pharmaceutical composition of any of the embodiments disclosed herein.

In one aspect, the present disclosure provides a method of treating or preventing inflammation of the integumentary system, the method comprising administering to a subject an mmRNA encoding the chimeric protein of any of the embodiments disclosed herein.

In one aspect, the present disclosure provides a method of treating or preventing an ailment caused by inflammation. In embodiments, the ailment is selected from psoriasis, psoriatic arthritis (PsA), plaque psoriasis, rheumatoid arthritis (RA), juvenile arthritis, ankylosing spondylitis, inflammatory bowel disease

(IBD), ulcerative colitis (UC), and Crohn's disease.

In embodiments, the inflammation is caused by or associated with a disease or disorder of the integumentary system. In embodiments, the inflammation is caused by or associated with a disease or disorder of the skin. In embodiments, the disease or disorder of the skin is psoriasis, pemphigus vulgaris, scleroderma, atopic dermatitis, sarcoidosis, erythema nodosum, hidradenitis suppurativa, lichen planus, Sweet's syndrome, vitiligo, chronic paronychia, eczema, seborrheic dermatitis, and/or hives. In embodiments, the disease or disorder of the skin is a psoriasis. In embodiments, the psoriasis is plaque psoriasis and/or psoriatic arthritis. Any aspect or embodiment described herein can be combined with any other aspect or embodiment as disclosed herein.

### BRIEF DESCRIPTION OF THE DRAWINGS

FIG. 1A shows schematic illustrations of the Type I transmembrane protein TNF receptor (TNFR2), which has an extracellular amino terminus and an intracellular carboxy terminus (left protein) and the Type II transmembrane proteins C-type lectin receptors (CLR) disclosed herein, which have an extracellular carboxy terminus and an intracellular amino terminus, (right protein). FIG. 1B and FIG. 1C show the illustrations of chimeric proteins of some aspects disclosed herein comprising the portions (e.g., the extracellular domains) of TNF receptor (TNFR2) and a C-type lectin receptor (CLR), with linkers connect the two. FIG. 1D and FIG. 1E show the illustrations of chimeric proteins of some aspects disclosed herein comprising the portions (e.g., the extracellular domains) of two C-type lectin receptors (CLR), with linkers connect the two.

FIG. 2A to FIG. 2E demonstrate the construction of the human TNFR2-Fc-Clec7a, TNFR2-Fc-DC-SIGN, TNFR2-Fc-Dectin2, and TNFR2-Fc-Langerin chimeric proteins. FIG. 2A shows a molecular weight ladder.

FIG. 2B are western blots of the TNFR2-Fc-Clec7a chimeric protein prosed with anti-TNFR2, anti-Fc and anti-Clec7a antibodies, showing the existence of all three parts in the chimeric protein. FIG. 2C are western blots of the TNFR2-Fc-Dectin2 chimeric protein prosed with anti-TNFR2, anti-Fc and anti-Dectin2 antibodies, showing the existence of all three parts in the chimeric protein. FIG. 2D are western blots of the TNFR2-Fc-DC-SIGN chimeric protein prosed with anti-TNFR2, anti-Fc and anti-DC-SIGN antibodies, showing the existence of all three parts in the chimeric protein. FIG. 2E are western blots of the TNFR2-Fc-Langerin chimeric protein prosed with anti-TNFR2, anti-Fc and anti-Langerin antibodies, showing the existence of all three parts in the chimeric protein.

FIG. 3A to FIG. 3G demonstrate the binding of the human TNFR2-Fc-Clec7a, TNFR2-Fc-DC-SIGN, TNFR2-Fc-Dectin2, and TNFR2-Fc-Langerin chimeric proteins to their respective ligands as measured using the Meso Scale Discovery (MSD) platform-based ELISA assays. FIG. 3A demonstrates the contemporaneous binding to an anti-TNFR antibody and an anti-Fc antibody by the human TNFR2-Fc-Clec7a, TNFR2-Fc-DC-SIGN, TNFR2-Fc-Dectin2, and TNER2-Fc-Langerin chimeric proteins. FIG. 3B demonstrates the contemporaneous binding to TNFa and an anti-Fc antibody by the human TNFR2-Fc-Clec7a, TNFR2-Fc-DC-SIGN, TNFR2-Fc-Dectin2, and TNER2-Fc-Langerin chimeric proteins. FIG. 3C demonstrates the contemporaneous binding by the human TNFR2-Fc-Clec7a chimeric protein to an anti-human Clec7a antibody and an anti-Fc antibody. FIG. 3D demonstrates the contemporaneous binding by the human TNFR2-Fc-DC-SIGN chimeric protein to an anti-human DC-SIGN antibody and an anti-Fc antibody. Human DC-SIGN-Fc and human TNFR2-Fc proteins were used as positive and negative controls, respectively. FIG. 3E demonstrates the contemporaneous binding by the human TNFR2-Fc-Dectin 2 chimeric protein to an anti-human Dectin2 antibody and an anti-Fc antibody. An irrelevant protein lacking human Dectin2 was used as negative control. FIG. 3F demonstrates the contemporaneous binding by the human TNFR2-Fc-Langerin chimeric protein to an anti-human langerin antibody and an anti-Fc antibody. Human langerin-Fc and human TNFR2-Fc proteins were used as positive and negative controls, respectively. FIG. 3G demonstrates the contemporaneous binding to laminarin and an anti-Fc antibody by the human TNFR2-Fc-Clec7a, TNFR2-Fc-DC-SIGN, TNFR2-Fc-Dectin2, and TNFR2-Fc-Langerin chimeric proteins.

FIG. 4A to FIG. 4D demonstrate the construction of the mouse TNFR2-Fc-Clec7a, TNFR2-Fc-DC-SIGN, TNFR2-Fc-Dectin2, and TNER2-Fc-Langerin chimeric proteins. FIG. 4A are Western blots showing characterization of the mouse TNFR2-Fc-Clec7 chimeric protein that are probed with an anti-TNFR2 antibody (left blot), an anti-Fc antibody (middle blot) and an anti-Clec7 antibody (right blot). FIG. 4B are Western blots showing characterization of the mouse TNFR2-Fc-Dectin2 chimeric protein that are probed with an anti-TNFR2 antibody (left blot), an anti-Fc antibody (middle blot) and an anti-Dectin2 antibody (right blot). FIG. 4C are Western blots showing characterization of the mouse TNFR2-Fc-DC-SIGN chimeric protein that are probed with an anti-TNFR2 antibody (left blot), an anti-Fc antibody (middle blot) and an anti-DC-SIGN antibody (right blot). FIG. 4D are Western blots showing characterization of the mouse TNFR2-Fc-Langerin chimeric protein that are probed with an anti-TNFR2 antibody (left blot), an anti-Fc antibody (middle blot) and an anti-Langerin antibody (right blot). The Western blots demonstrate the native state and tendency to form a multimer by the TNFR2-Fc-Clec7a, TNFR2-Fc-DC-SIGN, TNFR2-Fc-Dectin2, and TNFR2-Fc-Langerin chimeric proteins. Untreated samples (i.e., without a reducing agent or a deglycosylation agent, yet boiled) of the chimeric proteins, were loaded into lane 1 in all the blots. Samples in lane 2 were treated with a reducing agent, β-mercaptoethanol, and were boiled. Samples in lane 3 were treated with a deglycosylation agent, the reducing agent, and were boiled. A protein size ladder was included in each blot. The change in migration in lane 2 compared to lane 1 in each blot demonstrates oligomerization, and the change in migration in lane 3 compared to lane 2 in each blot demonstrates glycosylation.

FIG. 5A to FIG. 5N demonstrate the binding of the mouse TNFR2-Fc-Clec7a, TNFR2-Fc-DC-SIGN, TNFR2-Fc-Dectin2, and TNER2-Fc-Langerin chimeric proteins to their respective ligands as measured using the Meso Scale Discovery (MSD) platform-based ELISA assays. FIG. 5A demonstrates the contemporaneous binding to an anti-TNFR antibody and an anti-Fc antibody by the TNFR2-Fc-Clec7a, TNFR2-Fc-DC-SIGN, and TNFR2-Fc-Langerin chimeric proteins. FIG. 5B demonstrates the contemporaneous binding to TNFα and an anti-Fc antibody by the TNFR2-Fc-Clec7a, TNFR2-Fc-DC-SIGN, and TNFR2-Fc-Langerin chimeric proteins. FIG. 5C demonstrates the contemporaneous binding by the TNFR2-Fc-Clec7a chimeric protein to an anti-Clec7a antibody and an anti-Fc antibody. FIG. 5D demonstrates the contemporaneous binding by the TNFR2-Fc-DC-SIGN chimeric protein to an anti-DC-SIGN antibody and an anti-Fc antibody. FIG. 5E demonstrates the contemporaneous binding by the TNFR2-Fc-Dectin 2 chimeric protein to an anti-DC-SIGN antibody and an anti-Fc antibody. FIG. 5F demonstrates the contemporaneous binding by the TNFR2-Fc-Langerin chimeric protein to an anti-langerin antibody and an anti-Fc antibody. FIG. 5G demonstrates the contemporaneous binding to laminarin and an anti-Fc antibody by the TNFR2-Fc-Clec7a, TNFR2-Fc-DC-SIGN, TNFR2-Fc-Dectin2, and TNER2-Fc-Langerin chimeric proteins. FIG. 5H demonstrates the contemporaneous binding to galectin-9 and an anti-Fc antibody by the TNFR2-Fc-Clec7a, TNFR2-Fc-DC-SIGN, TNFR2-Fc-Dectin2, and TNFR2-Fc-Langerin chimeric proteins. FIG. 5I demonstrates the contemporaneous binding to dextran sulphate sodium (DSS) and an anti-Fc antibody by the TNFR2-Fc-Clec7a, TNFR2-Fc-DC-SIGN, TNFR2-Fc-Dectin2, and TNFR2-Fc-Langerin chimeric proteins. FIG. 5J demonstrates the contemporaneous binding to zymosan and an anti-Fc antibody by the TNFR2-Fc-Clec7a, TNFR2-Fc-DC-SIGN, TNFR2-Fc-Dectin2, and TNFR2-Fc-Langerin chimeric proteins. FIG. 5K demonstrates the contemporaneous binding to Inter-α-inhibitor heavy chain 4 (ITIH4) and an anti-Fc antibody by the TNFR2-Fc-DC-SIGN chimeric proteins. FIG. 5L demonstrates the contemporaneous binding to hyaluronan binding protein 1 (HABP1) and an anti-Fc antibody by the TNFR2-Fc-DC-SIGN chimeric proteins. FIG. 5M demonstrates the contemporaneous binding to CAECAM1 and an anti-Fc antibody by the TNFR2-Fc-DC-SIGN chimeric proteins. FIG. 5N demonstrates the contemporaneous binding to BTN2A1-His and an anti-Fc antibody by the TNFR2-Fc-DC-SIGN chimeric proteins.

FIG. 6 shows the detection of mRNA encoding the TNFR2-Fc-Clec7a, TNFR2-Fc-DC-SIGN, TNFR2-Fc-Dectin2, and TNFR2-Fc-Langerin chimeric proteins in cells using qPCR 24 hours after transfecting into CHOK1 cells.

FIG. 7A to FIG. 7C show the expression of the TNFR2-Fc-Clec7a, TNFR2-Fc-DC-SIGN, TNFR2-Fc-Dectin2, and TNFR2-Fc-Langerin chimeric proteins as measured using the Meso Scale Discovery (MSD) platform-based ELISA assays 24 hours after transfection with mRNA encoding the chimeric proteins in L929 (FIG. 7A), HEK293 (FIG. 7B), or CHOK1 cells (FIG. 7C).

FIG. 8A and FIG. 8B demonstrate that the chimeric proteins disclosed herein sequester their ligands and block the activation reporter cells. FIG. 8A shows the activation of secreted alkaline phosphatase (SEAP) reporter in HEK-Blue® Dectin2 cells were incubated with TNFa in the presence of the TNFR2-Fc-Clec7a or TNFR2-Fc-Dectin2 chimeric proteins or an irrelevant protein that was used as a negative control. FIG. 8B shows the activation of secreted alkaline phosphatase (SEAP) reporter in HEK-Blue® Dectin1b cells were incubated with the glycan/carbohydrate Zymosan in the presence of buffer alone, the TNFR2-Fc-Clec7a chimeric protein, or a negative control chimeric protein.

FIG. 9A and FIG. 9B demonstrate that the chimeric proteins disclosed herein block the TNFa-induced apoptosis of L929 fibroblast cells. FIG. 9A shows the blockade of apoptosis by mmRNA encoding the chimeric proteins disclosed herein. L929 fibroblast cells were transfected with empty LNP, or LNP comprising mmRNA encoding the TNFR2-Fc-Clec7a or TNFR2-Fc-Dectin2 chimeric proteins or an irrelevant chimeric protein, and then treated with increasing amounts of TNFa. The extent of apoptosis, as measured by cleaved caspase 3/7 activity, was plotted as a function of amount of TNFa. FIG. 9B shows the blockade of apoptosis by the chimeric proteins disclosed herein. L929 fibroblast cells were incubated with the TNFR2-Fc-Clec7a or TNFR2-Fc-Dectin2 chimeric proteins or an irrelevant chimeric protein, in the presence of increasing amounts of TNFa. The extent of apoptosis, as measured by cleaved caspase 3/7 activity, plotted as a function of the molar ratio of the chimeric protein: TNFα. The irrelevant chimeric protein that was used as a negative control.

FIG. 10A and FIG. 10B show the therapeutic activity of the chimeric proteins disclosed herein in vivo in a mouse model of colitis. FIG. 10A is a bar graph showing the change in body weight on day 8. FIG. 10B is a bar graph showing the proportion of CD3+CD45+CD4+ and CD3+CD45+CD8+ cells out of total CD4+/CD8+ cells.

### DETAILED DESCRIPTION

Disclosed herein are dual action chimeric proteins, and nucleic acids encoding the chimeric proteins, that, for instance, disrupt, block, reduce, and/or inhibit (1) the activity of TNFα, a cytokine that causes tissue inflammation, by, e.g., and without limitation sequestering TNFa and/or inhibiting the function of the cellular TNF receptor species; and (2) the transmission of overactive/aberrant sensing of pathogen associated molecular patterns (PAMPs) and damage associated molecular patterns (DAMPs, alarmins) cells (e.g., by sequestering the ligands that activate PAMPS/DAMPs receptors), and thereby preventing overactivation/aberrant activation of macrophages, monocytes and/or dendritic cells. Thus, without wishing to be bound by theory the chimeric proteins disclosed herein or a nucleic acid encoding the same provide an anti-inflammatory effect and/or an anti-autoimmune effect by two distinct pathways; this dual-action is more likely to provide a therapeutic effect in a patient and/or to provide an enhanced therapeutic effect in a patient.

Furthermore, without wishing to be bound by theory, since such chimeric proteins can act via two distinct pathways, they can be efficacious, at least, in patients who respond poorly to treatments that target one of the two pathways. Thus, a patient who is a poor responder to treatments acting via one of the two pathways can receive a therapeutic benefit by targeting the other pathway.

Disclosed herein is a chimeric protein, and a nucleic acid encoding the same, the chimeric protein comprising a first domain comprising a portion of TNF receptor (TNFR2) that is capable of binding TNFa and/or capable of oligomerizing with a cellular TNF receptor, which is connected via linker to a second domain comprising a portion of a C-type lectin receptor (CLR) capable of binding a ligand (without limitation, e.g., Clec7A, langerin, DC-SIGN, and Dectin-2).

The first domain and the second domain are present on the same polypeptide, thus (1) the chimeric protein disclosed herein are produced by a single transcript, and (2) unlike antibodies, heterodimerization of two polypeptides is not required. Therefore, the chimeric proteins disclosed herein may be delivered as purified protein or a nucleic acid encoding the chimeric protein disclosed herein. Accordingly, the present disclosure is also based, in part, on the delivery of a nucleic acid encoding the chimeric protein, e.g., to skin. The isolated polynucleotide encoding the chimeric protein disclosed herein and/or the chimeric protein disclosed herein may be used to treat a disease or disorder caused by or associated with inflammation of the integumentary system. The isolated polynucleotide encoding the chimeric protein and/or the chimeric protein disclosed herein may be delivered by any mode of administration suitable for treatment of the integumentary system, e.g., by topical administration.

### The Chimeric Proteins of the Present Disclosure

Transmembrane proteins typically consist of an extracellular domain, one or a series of transmembrane domains, and an intracellular domain. Without wishing to be bound by theory, the extracellular domain of a transmembrane protein is responsible for interacting with a soluble receptor or ligand or membrane-bound receptor or ligand (i.e., a membrane of an adjacent cell). Without wishing to be bound by theory, the transmembrane domain(s) is responsible for localizing the transmembrane protein to the plasma membrane. Without wishing to be bound by theory, the intracellular domain of a transmembrane protein is responsible for coordinating interactions with cellular signaling molecules to coordinate intracellular responses with the extracellular environment (or visa-versa). Thus, the transmembrane proteins may function as receptors (i.e.

initiate signal transduction in response to stimulation by a cognate ligand), ligands (i.e. stimulate signal transduction in the cells harboring a cognate receptor), or both as receptors and ligands (i.e. both stimulate signal transduction response binding of a cognate ligand and initiate signal transduction in the cells harboring a cognate receptor), depending on the context.

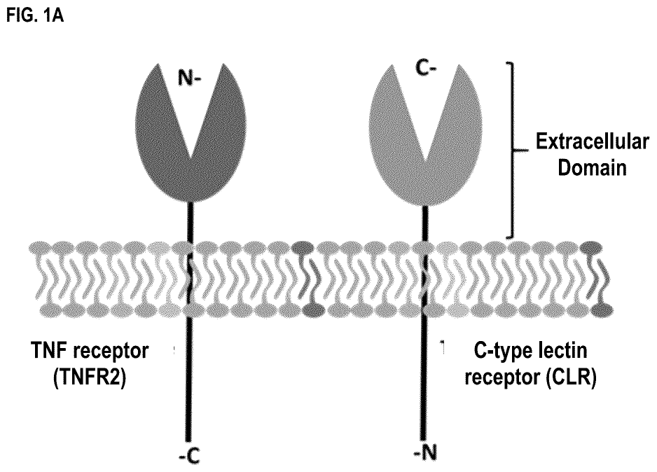

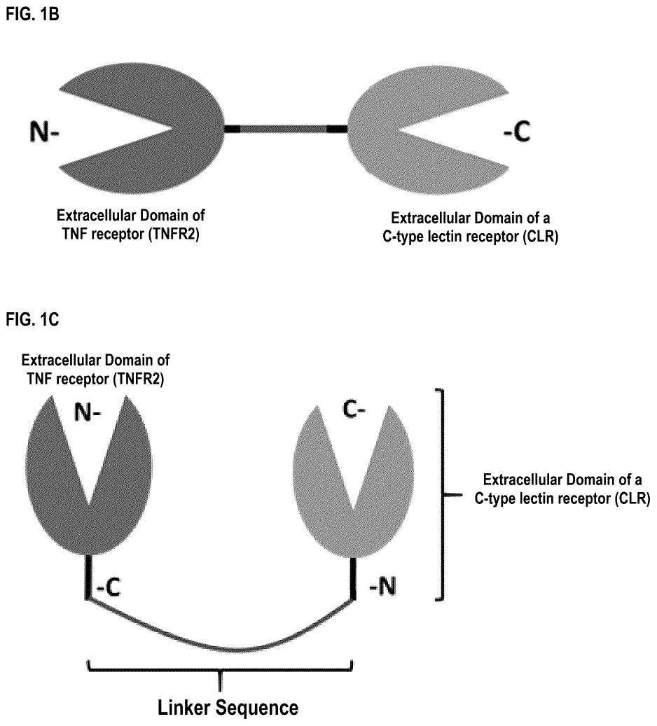

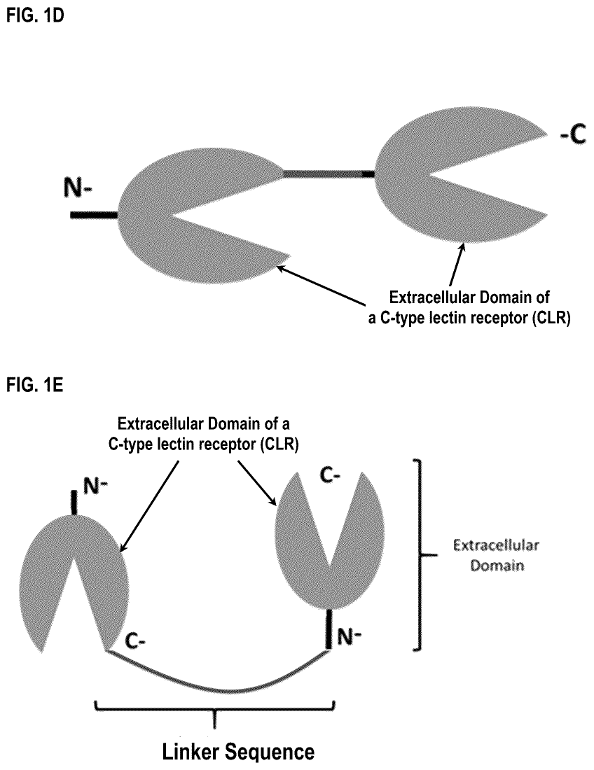

There are generally two types of single-pass transmembrane proteins: Type I transmembrane proteins which have an extracellular amino terminus and an intracellular carboxy terminus and Type II transmembrane proteins which have an extracellular carboxy terminus and an intracellular amino terminus (see, FIG. 1A, right protein). TNF receptor (TNFR2), which has an extracellular amino terminus and an intracellular carboxy terminus, is a Type I transmembrane protein (see, FIG. 1A, left protein). The C-type lectin receptors (CLR) disclosed herein, which have an extracellular carboxy terminus and an intracellular amino terminus, are Type II transmembrane proteins. For Type I transmembrane proteins (e.g., TNFR2) the amino terminus of the protein faces outside the cell, and therefore contains the functional domains that are responsible for interacting with other binding partners (either ligands or receptors, e.g., TNFα) in the extracellular environment. For Type II transmembrane proteins (e.g., the C-type lectin receptors (CLR) disclosed herein), the carboxy terminus of the protein faces outside the cell, and therefore contains the functional domains that are responsible for interacting with other binding partners (either ligands or receptors) in the extracellular environment. Thus, these two types of transmembrane proteins have opposite orientations to each other relative to the cell membrane, with the amino terminus of a Type I transmembrane protein is orientated away from the cell membrane whereas the amino terminus of a Type II transmembrane protein is orientated towards from the cell membrane.

In embodiments, an extracellular domain refers to a portion of a transmembrane protein, which is capable of interacting with the extracellular environment. In embodiments, an extracellular domain refers to a portion of a transmembrane protein, which is sufficient for binding to a ligand or receptor and is effective in transmitting a signal to a cell. In embodiments, an extracellular domain is the entire amino acid sequence of a transmembrane protein, which is normally present at the exterior of a cell or of the cell membrane. In embodiments, an extracellular domain is that portion of an amino acid sequence of a transmembrane protein which is external of a cell or of the cell membrane and is needed for signal transduction and/or ligand binding as may be assayed using methods know in the art (e.g., in vitro ligand binding and/or cellular activation assays).

In some aspects, the chimeric proteins of the present disclosure, comprise a Type I transmembrane protein (e.g., TNFR2) and a Type II transmembrane protein (e.g., the C-type lectin receptors (CLR) disclosed herein), which may be engineered such that their transmembrane and intracellular domains are omitted, and the transmembrane proteins' extracellular domains are adjoined using a linker sequence to generate a single chimeric protein. In embodiments, as shown in FIG. 1B and FIG. 1C, the extracellular domain of TNFR2 (a Type I transmembrane protein) and the extracellular domain of a C-type lectin receptors (CLR) disclosed herein (a Type II transmembrane protein) are combined into a single chimeric protein. FIG. 1B depicts some embodiments, where the linkage of a liberated TNFR2 (a Type I transmembrane protein, liberated from its transmembrane and intracellular domains) or a liberated carboxy-terminus anchored extracellular protein (from its anchoring domain) and a liberated a C-type lectin receptor (CLR) disclosed herein (a Type II transmembrane protein, liberated from its transmembrane and intracellular domains) have been adjoined by a linker sequence. The extracellular domains in this depiction may include the entire amino acid sequence of the TNFR2's extracellular domain, or a fraction thereof, wherein the fraction retains the ability to bind the TNFα. Likewise, the extracellular domains in this depiction may include the entire amino acid sequence of a C-type lectin receptors (CLR) disclosed herein, or a fraction thereof, wherein the fraction retains the ability to bind its intended ligand/receptor. Moreover, the chimeric protein of these aspects comprises sufficient overall flexibility and/or physical distance between domains such that a first extracellular domain (shown at the left end of the chimeric protein in FIG. 1B and FIG. 1C) is sterically capable of binding its receptor/ligand and/or a second extracellular domain (shown at the right end of the chimeric protein in FIG. 1B and FIG. 1C) is sterically capable of binding its receptor/ligand. FIG. 1C depicts adjoined extracellular domains in a linear chimeric protein wherein each extracellular domain of the chimeric protein is facing “outward”.

In some aspects, the chimeric proteins of the present disclosure comprise the extracellular domains of two C-type lectin receptors (CLR) disclosed herein (Type II transmembrane proteins), e.g., extracellular domains from two distinct a C-type lectin receptors (CLR) or two extracellular domains from one a C-type lectin receptor. Thus, a chimeric protein of those embodiments comprises, at least, a first domain comprising the extracellular domain of a first C-type lectin receptor (CLR), which is connected-directly or via a linker-to a second domain comprising the extracellular domain of a second C-type lectin receptor (CLR). As illustrated in FIG. 1D and FIG. 1E, when the domains are linked in an amino-terminal to carboxy-terminal orientation, the first domain is located on the “left” side of the chimeric protein and is “inward facing” and the second domain is located on “right” side of the chimeric protein and is “outward facing”.

Other configurations of first and second domains are envisioned, e.g., the first domain is outward facing and the second domain is inward facing, the first and second domains are both inward facing, and the first and second domains are both outward facing.

The present chimeric proteins and the nucleic acids encoding the present chimeric proteins provide advantages including, without limitation, ease of use and ease of production. This is because two distinct immunotherapy agents are combined into a single product which may allow for a single manufacturing process instead of two independent manufacturing processes. In addition, administration of a single agent instead of two separate agents allows for easier administration and greater patient compliance. Further, in contrast to, for example, monoclonal antibodies, which are large multimeric proteins containing numerous disulfide bonds and post-translational modifications such as glycosylation, the present chimeric proteins and the nucleic acids encoding the present chimeric proteins are easier and more cost effective to manufacture. Moreover, in contrast to, for example, monoclonal antibodies, which are made from two polypeptide chains that must be biosynthesized using at least two open reading frames, potentially from nucleic acids, the nucleic acids encoding the present chimeric proteins comprise a single polypeptide that is biosynthesized from a single nucleic acid molecule harboring a single open reading frame encoding the present chimeric proteins. Accordingly, and the present chimeric proteins may be suitably administered as purified chimeric proteins, or nucleic acids encoding the chimeric proteins.

In one aspect, the present disclosure provides a chimeric protein having a general structure of: N terminus-(a)-(b)-(c)-C terminus, or a nucleic acid encoding the same, wherein: (a) is a first domain comprising a portion of TNF receptor (TNFR2) that is capable of binding TNFa and/or capable of oligomerizing with a cellular TNF receptor; (b) is a linker adjoining the first domain and a second domain, optionally comprising a hinge-CH2-CH3 Fc domain, and (c) is a second domain comprising a portion of a C-type lectin receptor (CLR) capable of binding a ligand.

In one aspect, the present disclosure provides a chimeric protein, or a nucleic acid encoding the same, wherein the chimeric protein comprises a first domain comprising a portion of TNF receptor (TNFR2) that is capable of binding TNFa and/or capable of oligomerizing with a cellular TNF receptor, which is adjoined via a linker, which optionally comprises a hinge-CH2-CH3 Fc domain, to a second domain comprising a portion of a C-type lectin receptor (CLR). In embodiments, the portion of TNFR2 comprises the extracellular domain of TNFR2, or a fragment thereof, which is capable of binding TNFa and/or capable of oligomerizing with a cellular TNF receptor.

TNFR2 is single-spanning type I transmembrane proteins characterized by having four cysteine-rich domains (CRDs) in its extracellular domain. In embodiments, the extracellular domain of TNFR2, or a fragment thereof inhibits TNFa by competing with the cellular receptor species for TNF binding by sequestering. In embodiments, first domain inhibits TNFa by oligomerizing with cellular TNF receptor species, forming inactive complexes, and thereby inhibiting the function of the cellular TNF receptor species.

In embodiments, the first domain comprises the extracellular domain of TNFR2, which has the following sequence:

(SEQ ID NO: 57)
LPAQVAFTPYAPEPGSTCRLREYYDQTAQMCCSKCSPGQHAKVFCTKTS
DTVCDSCEDSTYTQLWNWVPECLSCGSRCSSDQVETQACTREQNRICTC
RPGWYCALSKQEGCRLCAPLRKCRPGFGVARPGTETSDWVCKPCAPGTF
SNTTSSTDICRPHQICNVVAIPGNASMDAVCTSTSPTRSMAPGAVHLPQ
PVSTRSQHTQPTPEPSTAPSTSFLLPMGPSPPAEGSTGD.

In embodiments, the chimeric protein comprises a variant of the extracellular domain of TNFR2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 57.

In embodiments, the portion of TNFR2 comprises the extracellular domain of TNFR2, or a fragment thereof. In embodiments, the portion of TNFR2 comprises an amino acid sequence that is at least about 90%, or at least about 95%, or at least about 97%, or at least about 98%, or at least about 99% identical to the amino acid sequence of SEQ ID NO: 57. In embodiments, the first domain of a chimeric protein comprises an amino acid sequence that is at least 95% identical to the amino acid sequence of SEQ ID NO: 57.

One of ordinary skill may select variants of the known amino acid sequence of TNFR2 by consulting literature and structural information, e.g., Kohno et al., “A second tumor necrosis factor receptor gene product can shed a naturally occurring tumor necrosis factor inhibitor.” Proc. Natl. Acad. Sci. U.S.A. 87 (21), 8331-8335 (1990); Smith et al., “A receptor for tumor necrosis factor defines an unusual family of cellular and viral proteins.” Science 248 (4958), 1019-1023 (1990); Loetscher et al., “Purification and partial amino acid sequence analysis of two distinct tumor necrosis factor receptors from HL60 cells.” J. Biol. Chem. 265 (33), 20131-20138 (1990); Dembic, et al., “Two human TNF receptors have similar extracellular, but distinct intracellular, domain sequences.” Cytokine 2 (4), 231-237 (1990); Pennica et al., “Biochemical properties of the 75-kDa tumor necrosis factor receptor. Characterization of ligand binding, internalization, and receptor phosphorylation.” J. Biol. Chem. 267 (29), 21172-21178 (1992); and Park et al., “Structural basis for self-association and receptor recognition of human TRAF2.” Nature 398 (6727), 533-538 (1999); Mukai et al., “Solution of the structure of the TNF-TNFR2 complex.” Sci Signal. 3 (148): ra83 (2010); TNF-TNFR2 structure PDB ID: 3ALQ, each of which is incorporated by reference in its entirety.

In one aspect, the present disclosure provides a chimeric protein or a nucleic acid encoding the same, wherein the chimeric protein comprises a first domain comprising a portion of TNF receptor (TNFR2), which is adjoined via a linker, which optionally comprises a hinge-CH2-CH3 Fc domain, to a second domain comprising a portion of a C-type lectin receptor (CLR) capable of binding a ligand. In embodiments, the ligand is a native ligand of the CLR. In embodiments, the CLR is selected from C-Type Lectin Domain Containing 7A(Clec7A), langerin, dendritic cell-specific intercellular adhesion molecule-3-grabbing non-integrin (DC-SIGN), and dendritic cell-associated C-type lectin-2 (Dectin-2).

In embodiments, the second domain in inhibits aberrant and/or overactivation of macrophages by blocking abnormal sensing of pathogen associated molecular patterns (PAMPs) and damage associated molecular patterns (DAMPs, alarmins). In embodiments, the ligand of a C-type lectin receptor (CLR) is PAMPs and/or DAMPs. In embodiments, the second domain in inhibits, reduces or blocks the initial recognition and uptake of PAMPs and/or DAMPs. In embodiments, the second domain in inhibits, reduces or blocks the macrophage-mediated inflammatory cascade, which is initiated by the initial recognition and uptake of PAMPs and/or DAMPs.

In embodiments, the second domain inhibits, blocks or reduces the initiation of the inflammatory responses that macrophages, monocytes, and/or dendritic cells initiate upon abnormal sensing of pathogen associated molecular patterns (PAMPs) and damage associated molecular patterns (DAMPs, alarmins), thereby blocks, reduced, and/or inhibits the production of pro-inflammatory cytokines such as TNFα, IL-17 and IL-23 by macrophages, monocytes, and/or dendritic cells.

In embodiments, the second domain comprises a portion of Clec7a (also known as dendritic cell-associated C-type lectin-1 (Dectin-1) or CD369). In embodiments, the portion of Clec7a comprises the extracellular domain of Clec7a, or a fragment thereof capable of binding a beta-1,3-linked and/or beta-1,6-linked glucan. Clec7a is a transmembrane protein containing a C-type lectin binding domain (CLD, also called carbohydrate-recognition domain, CRD) in the extracellular region (which recognizes beta-1,3-linked and/or beta-1,6-linked glucans and endogenous ligands on T cells). In embodiments, the second domain comprises the CLD.

In embodiments, the second domain comprises the extracellular domain (ECD) of Clec7a, which has the following sequence:

(SEQ ID NO: 58)
TMAIWRSNSGSNTLENGYFLSRNKENHSQPTQSSLEDSVTPTKAVKTTG
VLSSPCPPNWIIYEKSCYLFSMSLNSWDGSKRQCWQLGSNLLKIDSSNE
LGFIVKQVSSQPDNSFWIGLSRPQTEVPWLWEDGSTFSSNLFQIRTTAT
QENPSPNCVWIHVSVIYDQLCSVPSYSICEKKFSM.

In embodiments, the chimeric protein comprises a variant of the extracellular domain (ECD) of Clec7a. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the second domain comprises the C-type lectin binding domain (CLD) of Clec7a, which has the following sequence:

(SEQ ID NO: 59)
SSPCPPNWIIYEKSCYLFSMSLNSWDGSKRQCWQLGSNLLKIDSSNELG
FIVKQVSSQPDNSFWIGLSRPQTEVPWLWEDGSTFSSNLFQIRTTATQE
NPSPNCVWIHVSVIYDQLCSVPSYSICEKKFSM

In embodiments, the chimeric protein comprises a variant of the C-type lectin binding domain (CLD) of Clec7a. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 59.

In embodiments, the portion of Clec7a comprises the extracellular domain of Clec7a, or a fragment thereof. In embodiments, the portion of Clec7a comprises an amino acid sequence that is at least about 90%, or at least about 95%, or at least about 97%, or at least about 98%, or at least about 99% identical to the amino acid sequence of SEQ ID NO: 58 or SEQ ID NO: 59. In embodiments, the first domain of a chimeric protein comprises an amino acid sequence that is at least 95% identical to the amino acid sequence of SEQ ID NO: 58 or SEQ ID NO: 59.

One of ordinary skill may select variants of the known amino acid sequence of Clec7a by consulting the literature and structural information, e.g., Brown et al., Structure of the Fungal Beta-Glucan-Binding Immune Receptor Dectin-1: Implications for Function. Protein Sci 16:1042-1052 (2007); TNF-TNFR2 structure PDB ID: 2BPE; Alphafold structure (Jumper et al., Highly accurate protein structure prediction with AlphaFold. Nature 596:583-589 (2021)); Legentil et al., Molecular Interactions of β-(1—3)-Glucans with Their Receptors, Molecules 20 (6): 9745-66 (2015), each of which is incorporated by reference in its entirety.

In embodiments, the second domain comprises a portion of langerin (also known as C-type lectin domain family 4 member K or CD207). In embodiments, the portion of langerin comprises the extracellular domain of langerin, or a fragment thereof capable of binding a sulfated glycan, a mannosylated glycan, a keratan sulfate (KS) and/or a beta-glucan.

Langerin is a calcium-dependent lectin displaying mannose-binding specificity. It facilitates uptake of antigens and is involved in the routing and/or processing of antigen for presentation to T cells. Langerin is a major receptor on primary Langerhans cells for Candida species, Saccharomyces species, and Malassezia furfur. It binds to high-mannose structures present on the envelope glycoprotein of HIV virus, which is followed by subsequent targeting of the virus to the Birbeck granules leading to its rapid degradation.

In embodiments, the second domain comprises the extracellular domain (ECD) of langerin, which has the following sequence:

(SEQ ID NO: 60)
PRFMGTISDVKTNVQLLKGRVDNISTLDSEIKKNSDGMEAAGVQIQMVN
ESLGYVRSQFLKLKTSVEKANAQIQILTRSWEEVSTLNAQIPELKSDLE
KASALNTKIRALQGSLENMSKLLKRQNDILQVVSQGWKYFKGNFYYFSL
IPKTWYSAEQFCVSRNSHLTSVTSESEQEFLYKTAGGLIYWIGLTKAGM
EGDWSWVDDTPFNKVQSVRFWIPGEPNNAGNNEHCGNIKAPSLQAWNDA
PCDKTFLFICKRPYVPSEP.

In embodiments, the chimeric protein comprises a variant of the extracellular domain of langerin. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 60.

In embodiments, the second domain comprises the C-type lectin binding domain (CLD) of langerin, which has the following sequence:

(SEQ ID NO: 61)
QVVSQGWKYFKGNFYYFSLIPKTWYSAEQFCVSRNSHLTSVTSESEQEF
LYKTAGGLIYWIGLTKAGMEGDWSWVDDTPFNKVQSVRFWIPGEPNNAG
NNEHCGNIKAPSLQAWNDAPCDKTFLFICKRPYVPSEP

In embodiments, the chimeric protein comprises a variant of the C-type lectin binding domain (CLD) of langerin. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 61.

In embodiments, the portion of langerin comprises the extracellular domain of langerin, or a fragment thereof. In embodiments, the portion of langerin comprises an amino acid sequence that is at least about 90%, or at least about 95%, or at least about 97%, or at least about 98%, or at least about 99% identical to the amino acid sequence of SEQ ID NO: 60 or SEQ ID NO: 61. In embodiments, the first domain of a chimeric protein comprises an amino acid sequence that is at least 95% identical to the amino acid sequence of SEQ ID NO: 60 or SEQ ID NO: 61.

One of ordinary skill may select variants of the known amino acid sequence of langerin by consulting the literature and structural information, e.g., Nurisso et al., Structural studies of langerin and Birbeck granule: a macromolecular organization model, Biochemistry 48:2684-98 (2009); Feinberg et al., Trimeric structure of langerin. J. Biol. Chem. 285:13285-93 (2010); Feinberg et al., Structural basis for langerin recognition of diverse pathogen and mammalian glycans through a single binding site. J. Mol. Biol. 405:1027-39 (2011); Chatwell et al., The carbohydrate recognition domain of langerin reveals high structural similarity with the one of DC-SIGN but an additional, calcium-independent sugar-binding site, Mol. Immunol. 45:1981-94 (2008); Chabrol et al., Alteration of the langerin oligomerization state affects birbeck granule formation, Biophys. J. 108:666-77 (2015); Feinberg et al., Common polymorphisms in human langerin change specificity for glycan ligands. J. Biol. Chem. 288 (52): 36762-36771 (2013); Porkolab et al., Rational-Differential Design of Highly Specific Glycomimetic Ligands: Targeting DC-SIGN and Excluding Langerin Recognition. ACS Chem. Biol. 13 (3): 600-608 (2018); Alphafold structure (Jumper et al., Highly accurate protein structure prediction with AlphaFold. Nature 596:583-589 (2021)), each of which is incorporated by reference in its entirety.

In embodiments, the second domain comprises a portion of DC-SIGN (also known as C-type lectin domain family 4 member L or CD209). In embodiments, the portion of DC-SIGN comprises the extracellular domain of DC-SIGN, or a fragment thereof capable of binding an Intercellular Adhesion Molecule 2 (ICAM2) and/or Intercellular Adhesion Molecule 3 (ICAM3).

DC-SIGN is pathogen-recognition receptor expressed on the surface of immature dendritic cells (DCs) and involved in initiation of primary immune response. It is thought to mediate the endocytosis of pathogens which are subsequently degraded in lysosomal compartments.

In embodiments, the second domain comprises the extracellular domain of DC-SIGN, which has the following sequence:

(SEQ ID NO: 62)
QVSKVPSSISQEQSRQDAIYQNLTQLKAAVGELSEKSKLQEIYQELTQL
KAAVGELPEKSKLQEIYQELTRLKAAVGELPEKSKLQEIYQELTWLKAA
VGELPEKSKMQEIYQELTRLKAAVGELPEKSKQQEIYQELTRLKAAVGE
LPEKSKQQEIYQELTRLKAAVGELPEKSKQQEIYQELTQLKAAVERLCH
PCPWEWTFFQGNCYFMSNSQRNWHDSITACKEVGAQLVVIKSAEEQNFL
QLQSSRSNRFTWMGLSDLNQEGTWQWVDGSPLLPSFKQYWNRGEPNNVG
EEDCAEFSGNGWNDDKCNLAKFWICKKSAASCSRDEEQFLSPAPATPNP
PPA.

In embodiments, the chimeric protein comprises a variant of the extracellular domain (ECD) of DC-SIGN. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 62.

In embodiments, the second domain comprises the C-type lectin binding domain (CLD) of DC-SIGN, which has the following sequence:

(SEQ ID NO: 63)
HPCPWEWTFFQGNCYFMSNSQRNWHDSITACKEVGAQLVVIKSAEEQNF
LQLQSSRSNRFTWMGLSDLNQEGTWQWVDGSPLLPSFKQYWNRGEPNNV
GEEDCAEFSGNGWNDDKCNLAKFWICK

In embodiments, the chimeric protein comprises a variant of the C-type lectin binding domain (CLD) of DC-SIGN. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 63.

In embodiments, the portion of DC-SIGN comprises the extracellular domain of DC-SIGN, or a fragment thereof. In embodiments, the portion of DC-SIGN comprises an amino acid sequence that is at least about 90%, or at least about 95%, or at least about 97%, or at least about 98%, or at least about 99% identical to the amino acid sequence of SEQ ID NO: 62 or SEQ ID NO: 63. In embodiments, the first domain of a chimeric protein comprises an amino acid sequence that is at least 95% identical to the amino acid sequence of SEQ ID NO: 62 or SEQ ID NO: 63.

One of ordinary skill may select variants of the known amino acid sequence of DC-SIGN by consulting the literature and structural information, e.g., Feinberg et al., Structural basis for selective recognition of oligosaccharides by DC-SIGN and DC-SIGNR, Science 294:2163-6 (2001); Guo et al., Structural basis for distinct ligand-binding and targeting properties of the receptors DC-SIGN and DC-SIGNR, Nat. Struct. Mol. Biol. 11:591-8 (2004); Pokidysheva et al., Cryo-EM reconstruction of dengue virus in complex with the carbohydrate recognition domain of DC-SIGN, Cell 124:485-93 (2006); Feinberg et al., Multiple modes of binding enhance the affinity of DC-SIGN for high mannose N-linked glycans found on viral glycoproteins. J. Biol. Chem. 282 4202-9 (2007); Thepaut et al., Structure of a glycomimetic ligand in the carbohydrate recognition domain of C-type lectin DC-SIGN. Structural requirements for selectivity and ligand design. J. Am. Chem. Soc. 135 2518-29 (2013); Medve et al., Enhancing potency and selectivity of a DC-SIGN glycomimetic ligand by fragment-based design: structural basis. Chemistry 25 (64): 14659-14668 (2019); Sutkeviciute et al., Unique DC-SIGN Clustering Activity of a Small Glycomimetic: A Lesson for Ligand Design. ACS Chem. Biol. 9 (6): 1377-1385 (2014); Porkolab et al., Rational-Differential Design of Highly Specific Glycomimetic Ligands: Targeting DC-SIGN and Excluding Langerin Recognition. ACS Chem. Biol. 13 (3): 600-608 (2018); Alphafold structure (Jumper et al., Highly accurate protein structure prediction with AlphaFold. Nature 596:583-589 (2021)), each of which is incorporated by reference in its entirety.

In embodiments, the second domain comprises a portion of Dectin-2 (also known as C-type lectin domain family 6 member A or C-type lectin superfamily member 10). In embodiments, the portion of Dectin-2 comprises the extracellular domain of Dectin-2, or a fragment thereof capable of binding an alpha-mannan.

Dectin-2 is a calcium-dependent lectin that acts as a pattern recognition receptor (PRR) of the innate immune system: specifically recognizes and binds alpha-mannans on C. albicans hypheas. In embodiments, the portion of Dectin-2 comprises the extracellular domain of Dectin-2, or a fragment thereof capable of recognizing allergens from house dust mite and fungi in a mannose-dependent manner, and/or soluble elements from the eggs of Shistosoma mansoni altering adaptive immune responses.

In embodiments, the second domain comprises the extracellular domain of Dectin-2, which has the following sequence:

(SEQ ID NO: 64)
TYHFTYGETGKRLSELHSYHSSLTCFSEGTKVPAWGCCPASWKSFGSSC
YFISSEEKVWSKSEQNCVEMGAHLVVFNTEAEQNFIVQQLNESFSYFLG
LSDPQGNNNWQWIDKTPYEKNVRFWHLGEPNHSAEQCASIVFWKPTGWG
WNDVICETRRNSICEMNKIYL.

In embodiments, the chimeric protein comprises a variant of the extracellular domain of Dectin-2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 64.

In embodiments, the second domain comprises the C-type lectin binding domain (CLD) of Dectin-2, which has the following sequence:

(SEQ ID NO: 65)
FGSSCYFISSEEKVWSKSEQNCVEMGAHLVVFNTEAEQNFIVQQLNESF
SYFLGLSDPQGNNNWQWIDKTPYEKNVRFWHLGEPNHSAEQCASIVFWK
PTGWGWNDVICETRRNSICE

In embodiments, the chimeric protein comprises a variant of the C-type lectin binding domain (CLD) of Dectin-2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 65.

In embodiments, the portion of Dectin-2 comprises the extracellular domain of Dectin-2, or a fragment thereof. In embodiments, the portion of Dectin-2 comprises an amino acid sequence that is at least about 90%, or at least about 95%, or at least about 97%, or at least about 98%, or at least about 99% identical to the amino acid sequence of SEQ ID NO: 64 or SEQ ID NO: 65. In embodiments, the first domain of a chimeric protein comprises an amino acid sequence that is at least 95% identical to the amino acid sequence of SEQ ID NO: 64 or SEQ ID NO: 65.

One of ordinary skill may select variants of the known amino acid sequence of Dectin-2 by consulting the literature and structural information, e.g., Feinberg et al., Mechanism of pathogen recognition by human dectin-2. J. Biol. Chem. 292 (32): 13402-13414 (2017); Decout et al., Deciphering the molecular basis of mycobacteria and lipoglycan recognition by the C-type lectin Dectin-2, Scientific Reports 8:16840 (2018); McGreal et al., The carbohydrate-recognition domain of Dectin-2 is a C-type lectin with specificity for high mannose, Glycobiology, 16 (5): 422-430 (2006); Alphafold structure (Jumper et al., Highly accurate protein structure prediction with AlphaFold. Nature 596:583-589 (2021)), each of which is incorporated by reference in its entirety.

In one aspect, the present disclosure provides a chimeric protein or a nucleic acid encoding the same, wherein the chimeric protein comprises a first domain comprising a portion of a first C-type lectin receptor (CLR) capable of binding a ligand, which is adjoined via a linker, which optionally comprises a hinge-CH2-CH3 Fc domain, to a second domain comprising a portion of a second C-type lectin receptor (CLR) capable of binding a ligand. In embodiments, the ligand is a native ligand of the CLR. In embodiments, the first CLR and the second CLR are independently selected from C-Type Lectin Domain Containing 7A(Clec7A), langerin, dendritic cell-specific intercellular adhesion molecule-3-grabbing non-integrin (DC-SIGN), and dendritic cell-associated C-type lectin-2 (Dectin-2). In embodiments, the first CLR and the second CLR independently comprise an extracellular domain (ECD) or a C-type lectin binding domain (CLD).

In one aspect, the present disclosure provides a chimeric protein having a general structure of: N terminus-(a)-(b)-(c)-C terminus, or a nucleic acid encoding the same, wherein: (a) is a first domain comprising a portion of a first C-type lectin receptor (CLR) capable of binding its ligand; (b) is a linker adjoining the first domain and a second domain comprising a hinge-CH2-CH3 Fc domain, and (c) is the second domain comprising a portion of a second C-type lectin receptor (CLR) capable of binding its ligand.

In embodiments, the portion of the first C-type lectin receptor (CLR) comprises the extracellular domain (ECD) of Clec7a, which has an amino acid sequence that is at least about 90%, or at least about 91%, or at least about 92%, or at least about 93%, or at least about 94%, or at least about 95%, or at least about 96%, or at least about 97%, or at least about 98%, or at least about 99% sequence identity to the amino acid sequence of SEQ ID NO: 58. In embodiments, the portion of the first C-type lectin receptor (CLR) comprises the C-type lectin binding domain (CLD) of Clec7a, which has an amino acid sequence that is at least about 90%, or at least about 91%, or at least about 92%, or at least about 93%, or at least about 94%, or at least about 95%, or at least about 96%, or at least about 97%, or at least about 98%, or at least about 99% sequence identity to the amino acid sequence of SEQ ID NO: 59.

In embodiments, the portion of the second C-type lectin receptor (CLR) comprises the extracellular domain (ECD) of Clec7a, which has an amino acid sequence that is at least about 90%, or at least about 91%, or at least about 92%, or at least about 93%, or at least about 94%, or at least about 95%, or at least about 96%, or at least about 97%, or at least about 98%, or at least about 99% sequence identity to the amino acid sequence of SEQ ID NO: 58. In embodiments, the portion of the second C-type lectin receptor (CLR) comprises the C-type lectin binding domain (CLD) of Clec7a, which has an amino acid sequence that is at least about 90%, or at least about 91%, or at least about 92%, or at least about 93%, or at least about 94%, or at least about 95%, or at least about 96%, or at least about 97%, or at least about 98%, or at least about 99% sequence identity to the amino acid sequence of SEQ ID NO: 59.

In embodiments, the portion of the first C-type lectin receptor (CLR) comprises the extracellular domain (ECD) of langerin, which has an amino acid sequence that is at least about 90%, or at least about 91%, or at least about 92%, or at least about 93%, or at least about 94%, or at least about 95%, or at least about 96%, or at least about 97%, or at least about 98%, or at least about 99% sequence identity to the amino acid sequence of SEQ ID NO: 60. In embodiments, the portion of the first C-type lectin receptor (CLR) comprises the C-type lectin binding domain (CLD) of langerin, which has an amino acid sequence that is at least about 90%, or at least about 91%, or at least about 92%, or at least about 93%, or at least about 94%, or at least about 95%, or at least about 96%, or at least about 97%, or at least about 98%, or at least about 99% sequence identity to the amino acid sequence of SEQ ID NO: 61.

In embodiments, the portion of the second C-type lectin receptor (CLR) comprises the extracellular domain (ECD) of langerin, which has an amino acid sequence that is at least about 90%, or at least about 91%, or at least about 92%, or at least about 93%, or at least about 94%, or at least about 95%, or at least about 96%, or at least about 97%, or at least about 98%, or at least about 99% sequence identity to the amino acid sequence of SEQ ID NO: 60. In embodiments, the portion of the second C-type lectin receptor (CLR) comprises the C-type lectin binding domain (CLD) of langerin, which has an amino acid sequence that is at least about 90%, or at least about 91%, or at least about 92%, or at least about 93%, or at least about 94%, or at least about 95%, or at least about 96%, or at least about 97%, or at least about 98%, or at least about 99% sequence identity to the amino acid sequence of SEQ ID NO: 61.

In embodiments, the portion of the first C-type lectin receptor (CLR) comprises the extracellular domain (ECD) of DC-SIGN, which has an amino acid sequence that is at least about 90%, or at least about 91%, or at least about 92%, or at least about 93%, or at least about 94%, or at least about 95%, or at least about 96%, or at least about 97%, or at least about 98%, or at least about 99% sequence identity to the amino acid sequence of SEQ ID NO: 62. In embodiments, the portion of the first C-type lectin receptor (CLR) comprises the C-type lectin binding domain (CLD) of DC-SIGN, which has an amino acid sequence that is at least about 90%, or at least about 91%, or at least about 92%, or at least about 93%, or at least about 94%, or at least about 95%, or at least about 96%, or at least about 97%, or at least about 98%, or at least about 99% sequence identity to the amino acid sequence of SEQ ID NO: 63.

In embodiments, the portion of the second C-type lectin receptor (CLR) comprises the extracellular domain (ECD) of DC-SIGN, which has an amino acid sequence that is at least about 90%, or at least about 91%, or at least about 92%, or at least about 93%, or at least about 94%, or at least about 95%, or at least about 96%, or at least about 97%, or at least about 98%, or at least about 99% sequence identity to the amino acid sequence of SEQ ID NO: 62. In embodiments, the portion of the second C-type lectin receptor (CLR) comprises the C-type lectin binding domain (CLD) of DC-SIGN, which has an amino acid sequence that is at least about 90%, or at least about 91%, or at least about 92%, or at least about 93%, or at least about 94%, or at least about 95%, or at least about 96%, or at least about 97%, or at least about 98%, or at least about 99% sequence identity to the amino acid sequence of SEQ ID NO: 63.

In embodiments, the portion of the first C-type lectin receptor (CLR) comprises the extracellular domain (ECD) of Dectin-2, which has an amino acid sequence that is at least about 90%, or at least about 91%, or at least about 92%, or at least about 93%, or at least about 94%, or at least about 95%, or at least about 96%, or at least about 97%, or at least about 98%, or at least about 99% sequence identity to the amino acid sequence of SEQ ID NO: 64. In embodiments, the portion of the first C-type lectin receptor (CLR) comprises the C-type lectin binding domain (CLD) of Dectin-2, which has an amino acid sequence that is at least about 90%, or at least about 91%, or at least about 92%, or at least about 93%, or at least about 94%, or at least about 95%, or at least about 96%, or at least about 97%, or at least about 98%, or at least about 99% sequence identity to the amino acid sequence of SEQ ID NO: 65.

In embodiments, the portion of the second C-type lectin receptor (CLR) comprises the extracellular domain (ECD) of Dectin-2, which has an amino acid sequence that is at least about 90%, or at least about 91%, or at least about 92%, or at least about 93%, or at least about 94%, or at least about 95%, or at least about 96%, or at least about 97%, or at least about 98%, or at least about 99% sequence identity to the amino acid sequence of SEQ ID NO: 64. In embodiments, the portion of the second C-type lectin receptor (CLR) comprises the C-type lectin binding domain (CLD) of Dectin-2, which has an amino acid sequence that is at least about 90%, or at least about 91%, or at least about 92%, or at least about 93%, or at least about 94%, or at least about 95%, or at least about 96%, or at least about 97%, or at least about 98%, or at least about 99% sequence identity to the amino acid sequence of SEQ ID NO: 65.

In embodiments, a heterologous chimeric protein of the present disclosure comprises, or the isolated polynucleotide encoding the heterologous chimeric protein encodes, the extracellular domain of human CD69, which comprises the following amino acid sequence:

(SEQ ID NO: 80)
LPAQVAFTPYAPEPGSTCRLREYYDQTAQMCCSKCSPGQHAKVFCTKTS
DTVCDSCEDSTYTQLWNWVPECLSCGSRCSSDQVETQACTREQNRICTC
RPGWYCALSKQEGCRLCAPLRKCRPGFGVARPGTETSDVVCKPCAPGTF
SNTTSSTDICRPHQICNVVAIPGNASMDAVCTSTSPTRSMAPGAVHLPQ
PVSTRSQHTQPTPEPSTAPSTSFLLPMGPSPPAEGSTGD.

In embodiments, a heterologous chimeric protein used in methods of the present disclosure comprises a variant of the extracellular domain of CD69. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 80.

In embodiments, the first domain of a heterologous chimeric protein comprises an amino acid sequence that is at least 95% identical to the amino acid sequence of SEQ ID NO: 80.

In embodiments, a heterologous chimeric protein comprises substantially the entire extracellular domain of CD69.

One of ordinary skill may select variants of the known amino acid sequence of CD69 by consulting the literature, e.g., Llera et al., Crystal Structure of the C-Type Lectin-Like Domain from the Human Hematopoietic Cell Receptor CD69, J Biol Chem 276 (10): 7312-7319 (2001); Natarajan et al., Crystal structure of human CD69: a C-type lectin-like activation marker of hematopoietic cells, Biochemistry 39:14779-14786 (2000); Vanek et al., Soluble recombinant CD69 receptors optimized to have an exceptional physical and chemical stability display prolonged circulation and remain intact in the blood of mice, FEBS J 275:5589-5606 (2008); Kolenko et al., The high-resolution structure of the extracellular domain of human CD69 using a novel polymer, Acta Crystallogr Sect F Struct Biol Cryst Commun 65:1258-1260 (2009), each of which is incorporated by reference in its entirety.

### The Linker

In one aspect, the present disclosure provides a chimeric protein comprising a portion of TNF receptor (TNFR2) and a portion of a C-type lectin receptor (CLR) adjoined by a linker, or a nucleic acid encoding the chimeric protein. In embodiments, the linker comprises at least one cysteine residue capable of forming a disulfide bond. The at least one cysteine residue is capable of forming a disulfide bond between a pair (or more) of chimeric proteins. Without wishing to be bound by theory, such disulfide bond forming is responsible for maintaining a useful multimeric state of chimeric proteins. This allows for efficient production of the chimeric proteins; it allows for desired activity in vitro and in vivo.

In embodiments, the linker is not a single amino acid linker, e.g., without limitation, the linker is greater than one amino acid long. In embodiments, the linker has a length of greater than 1-6 amino acids, e.g., without limitation, the linker is greater than seven amino acids long. In embodiments, the linker comprises more than a single glycine residue.

In embodiments, in a chimeric protein of the present disclosure, the linker is a polypeptide selected from a flexible amino acid sequence, an IgG hinge region, or an antibody sequence. In embodiments, the linker comprises hinge-CH2-CH3 Fc domain derived from IgG4, optionally human IgG4. In embodiments, the linker comprises hinge-CH2-CH3 Fc domain derived from IgG1, optionally human IgG1.

In embodiments, the linker may be derived from naturally-occurring multi-domain proteins or is an empirical linker as described, for example, in Chichili et al., (2013), Protein Sci. 22 (2): 153-167, Chen et al., (2013), Adv Drug Deliv Rev. 65 (10): 1357-1369, the entire contents of which are hereby incorporated by reference. In embodiments, the linker may be designed using linker designing databases and computer programs such as those described in Chen et al., (2013), Adv Drug Deliv Rev. 65 (10): 1357-1369 and Crasto et. al., (2000), Protein Eng. 13 (5): 309-312, the entire contents of which are hereby incorporated by reference.

In embodiments, the linker is a synthetic linker such as PEG.

In embodiments, the linker comprises a polypeptide. In embodiments, the polypeptide is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In embodiments, the linker is flexible.

In embodiments, the linker is rigid.

In embodiments, the linker is substantially comprised of glycine and serine residues (e.g., about 30%, or about 40%, or about 50%, or about 60%, or about 70%, or about 80%, or about 90%, or about 95%, or about 97%, or about 98%, or about 99%, or about 100% glycines and serines).

In embodiments, the linker comprises a hinge region of an antibody (e.g., of IgG, IgA, IgD, and IgE, inclusive of subclasses (e.g., IgG1, IgG2, IgG3, and IgG4, and IgA1, and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. In embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of CH1 to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the C_{H2} domain and includes residues in C_{H2}. Id. The core hinge region of wild-type human IgG1 contains the sequence CPPC (SEQ ID NO: 24) which, when dimerized by disulfide bond formation, results in a cyclic octapeptide believed to act as a pivot, thus conferring flexibility. In embodiments, the present linker comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In embodiments, the linker of the present disclosure comprises one or more glycosylation sites.

In embodiments, the linker comprises an Fc domain of an antibody (e.g., of IgG, IgA, IgD, and IgE, inclusive of subclasses (e.g., IgG1, IgG2, IgG3, and IgG4, and IgA1 and IgA2).

In a chimeric protein of the present disclosure, the linker comprises a hinge-C_{H2}-C_{H3} Fc domain derived from IgG4. In embodiments, the linker comprises a hinge-C_{H2}-C_{H3} Fc domain derived from a human IgG4. In embodiments, the linker has at least about 95%, or at least about 97%, or at least about 97%, or at least about 98% sequence identity with the amino acid sequence of any one of SEQ ID NO: 1 to SEQ ID NO: 3 or SEQ ID NO: 73, e.g., at least 95% identical to the amino acid sequence of SEQ ID NO: 73. In embodiments, the linker comprises one or more joining linkers, such joining linkers independently selected from SEQ ID NOs: 4-50 (or a variant thereof). In embodiments, the linker comprises two or more joining linkers each joining linker independently selected from SEQ ID NOs: 4-50 (or a variant thereof); wherein one joining linker is N terminal to the hinge-C_{H2}-C_{H3} Fc domain and another joining linker is C terminal to the hinge-C_{H2}-C_{H3} Fc domain.

In embodiments, the linker comprises a hinge-C_{H2}-C_{H3} Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the present chimeric proteins.

In embodiments, the Fc domain in a linker contains one or more amino acid substitutions at amino acid residue 250, 252, 254, 256, 308, 309, 311, 416, 428, 433 or 434 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference), or equivalents thereof. In embodiments, the amino acid substitution at amino acid residue 250 is a substitution with glutamine. In embodiments, the amino acid substitution at amino acid residue 252 is a substitution with tyrosine, phenylalanine, tryptophan or threonine. In embodiments, the amino acid substitution at amino acid residue 254 is a substitution with threonine. In embodiments, the amino acid substitution at amino acid residue 256 is a substitution with serine, arginine, glutamine, glutamic acid, aspartic acid, or threonine. In embodiments, the amino acid substitution at amino acid residue 308 is a substitution with threonine. In embodiments, the amino acid substitution at amino acid residue 309 is a substitution with proline. In embodiments, the amino acid substitution at amino acid residue 311 is a substitution with serine. In embodiments, the amino acid substitution at amino acid residue 385 is a substitution with arginine, aspartic acid, serine, threonine, histidine, lysine, alanine or glycine. In embodiments, the amino acid substitution at amino acid residue 386 is a substitution with threonine, proline, aspartic acid, serine, lysine, arginine, isoleucine, or methionine. In embodiments, the amino acid substitution at amino acid residue 387 is a substitution with arginine, proline, histidine, serine, threonine, or alanine. In embodiments, the amino acid substitution at amino acid residue 389 is a substitution with proline, serine or asparagine. In embodiments, the amino acid substitution at amino acid residue 416 is a substitution with serine. In embodiments, the amino acid substitution at amino acid residue 428 is a substitution with leucine. In embodiments, the amino acid substitution at amino acid residue 433 is a substitution with arginine, serine, isoleucine, proline, or glutamine. In embodiments, the amino acid substitution at amino acid residue 434 is a substitution with histidine, phenylalanine, or tyrosine.

In embodiments, the Fc domain linker (e.g., comprising an IgG constant region) comprises one or more mutations such as substitutions at amino acid residue 252, 254, 256, 433, 434, or 436 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). In embodiments, the IgG constant region includes a triple M252Y/S254T/T256E mutation or YTE mutation. In embodiments, the IgG constant region includes a triple H433K/N434F/Y436H mutation or KFH mutation. In embodiments, the IgG constant region includes an YTE and KFH mutation in combination.

In embodiments, the linker comprises an IgG constant region that contains one or more mutations at amino acid residues 250, 253, 307, 310, 380, 428, 433, 434, and 435 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). Illustrative mutations include T250Q, M428L, T307A, E380A, 1253A, H310A, M428L, H433K, N434A, N434F, N434S, and H435A.

In embodiments, the IgG constant region comprises a M428L/N434S mutation or LS mutation. In embodiments, the IgG constant region comprises a T250Q/M428L mutation or QL mutation. In embodiments, the IgG constant region comprises an N434A mutation. In embodiments, the IgG constant region comprises a T307A/E380A/N434A mutation or AAA mutation. In embodiments, the IgG constant region comprises an 1253A/H310A/H435A mutation or IHH mutation. In embodiments, the IgG constant region comprises a H433K/N434F mutation. In embodiments, the IgG constant region comprises a M252Y/S254T/T256E and a H433K/N434F mutation in combination.

Additional exemplary mutations in the IgG constant region are described, for example, in Robbie, et al., Antimicrobial Agents and Chemotherapy (2013), 57 (12): 6147-6153, Dall'Acqua et al., JBC (2006), 281 (33): 23514-24, Dall'Acqua et al., Journal of Immunology (2002), 169:5171-80, Ko et al. Nature (2014) 514:642-645, Grevys et al. Journal of Immunology. (2015), 194 (11): 5497-508, and U.S. Pat. No. 7,083,784, the entire contents of which are hereby incorporated by reference.

In embodiments, the Fc domain in a linker comprises the amino acid sequence of SEQ ID NO: 73 (see the below table), or at least about 90%, or at least about 93%, or at least about 95%, or at least about 97%, or at least about 98%, or at least about 99% identity thereto. In embodiments, the Fc domain in a linker comprises the amino acid sequence of SEQ ID NO: 1 (see the below table), or at least about 90%, or at least about 93%, or at least about 95%, or at least about 97%, or at least about 98%, or at least about 99% identity thereto. In embodiments, mutations are made to SEQ ID NO: 1 to increase stability and/or half-life. For instance, in embodiments, the Fc domain in a linker comprises the amino acid sequence of SEQ ID NO: 2 (see the below table), or at least about 90%, or at least about 93%, or at least about 95%, or at least about 97%, or at least about 98%, or at least about 99% identity thereto. An illustrative Fc stabilizing mutant is S228P. Illustrative Fc half-life extending mutants are T250Q, M428L, V308T, L309P, and Q311S and the present linkers may comprise 1, or 2, or 3, or 4, or 5 of these mutants.

In embodiments, the chimeric protein binds to FcRn with high affinity. In embodiments, the chimeric protein may bind to FcRn with a Ko of about 1 nM to about 80 nM. For example, the chimeric protein may bind to FcRn with a Ko of about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 71 nM, about 72 nM, about 73 nM, about 74 nM, about 75 nM, about 76 nM, about 77 nM, about 78 nM, about 79 nM, or about 80 nM. In embodiments, the chimeric protein may bind to FcRn with a K_{D} of about 9 nM. In embodiments, the chimeric protein does not substantially bind to other Fc receptors (i.e. other than FcRn) with effector function.

In embodiments, the Fc domain in a linker has the amino acid sequence of SEQ ID NO: 1 (see Table 1, below), or at least at least about 90%, or at least about 93%, or at least about 95%, or at least about 97%, or at least about 98%, or at least about 99% identity thereto. In embodiments, mutations are made to SEQ ID NO: 1 to increase stability and/or half-life. For instance, in embodiments, the Fc domain in a linker comprises the amino acid sequence of SEQ ID NO: 2 (see Table 1, below), or at least at least about 90%, or at least about 93%, or at least about 95%, or at least about 97%, or at least about 98%, or at least about 99% identity thereto. For instance, in embodiments, the Fc domain in a linker comprises the amino acid sequence of SEQ ID NO: 3 (see Table 1, below), or at least at least about 90%, or at least about 93%, or at least about 95%, or at least about 97%, or at least about 98%, or at least about 99% identity thereto. For instance, in embodiments, the Fc domain in a linker comprises the amino acid sequence of SEQ ID NO: 73 (see Table 1, below), or at least at least about 90%, or at least about 93%, or at least about 95%, or at least about 97%, or at least about 98%, or at least about 99% identity thereto.

In embodiments, the hinge-C_{H2}-C_{H3} Fc domain is derived from IgG1. For instance, in embodiments, the Fc domain in a linker comprises the amino acid sequence of SEQ ID NO: 73 (see Table 1, below), or at least at least about 90%, or at least about 93%, or at least about 95%, or at least about 97%, or at least about 98%, or at least about 99% identity thereto. In embodiments, the IgG1 is human IgG1. In embodiments, the hinge-C_{H2}-C_{H3} Fc domain is derived from IgG4. In embodiments, the IgG4 is human IgG4. In embodiments, the hinge-C_{H2}-C_{H3} Fc domain comprises an amino acid sequence that is at least about 95% identical to the amino acid sequence of SEQ ID NO: 1, SEQ ID NO: 2, SEQ ID NO: 3, or SEQ ID NO: 73. In embodiments, the linker further comprises the linker comprises one or more joining linkers, such joining linkers independently selected from SEQ ID NOs: 4 to 50. In embodiments, the linker comprises two or more joining linkers each joining linker independently selected from SEQ ID NOs: 4 to 50; wherein one joining linker is N terminal to the hinge-C_{H2}-C_{H3}-Fc domain and another joining linker is C terminal to the hinge-C_{H2}-C_{H3}-Fc domain.

In embodiments, the Fc domain comprises a mammalian Fc domain. In embodiments, the Fc domain comprises a Fc domain selected from a human Fc domain, a mouse Fc domain, a rat Fc domain, a sheep Fc domain, a goat Fc domain, a donkey Fc domain, a feline Fc domain a hamster Fc domain, a guinea pig

Fc domain, a horse Fc domain, a rabbit Fc domain, a pig Fc domain, a dog Fc domain, and a cow Fc domain. In embodiments, the Fc domain comprises a human Fc domain, optionally comprising one or more mutations that increase serum half-life (e.g. M252Y, S254T, and T256E), enhance dimerization (e.g. S228P or knob in hole mutations), decrease Fc effector function (e.g. L234A and L235A mutations (LALA) with or without P329G mutation), and/or enhanced binding to the neonatal Fc receptor (FcRn). In embodiments, the Fc domain in a linker comprises the amino acid sequence of SEQ ID NO: 73 (see Table 1, below), or at least at least about 90%, or at least about 93%, or at least about 95%, or at least about 97%, or at least about 98%, or at least about 99% identity thereto. In embodiments, the Fc domain comprises a Fc domain selected from an IgG Fc domain, an IgA Fc domain an IgM Fc domain, an IgE Fc domain and an IgD Fc domain. In embodiments, the IgG Fc domain is selected from an IgG1 Fc domain, an IgG2 Fc domain, an IgG3 Fc domain, and an IgG4 Fc domain. In embodiments, the IgA is selected from an IgA1 and an IgA2.

Further, one or more joining linkers may be employed to connect an Fc domain in a linker (e.g., one of SEQ ID NO: 1, SEQ ID NO: 2, SEQ ID NO: 3, SEQ ID NO: 73, or at least at least about 90%, or at least about 93%, or at least about 95%, or at least about 97%, or at least about 98%, or at least about 99% identity thereto) and the extracellular domains. For example, any one of SEQ ID NO: 4, SEQ ID NO: 5, SEQ ID NO: 6, SEQ ID NO: 7, SEQ ID NO: 8, SEQ ID NO: 9, or variants thereof may connect an extracellular domain as disclosed herein and an Fc domain in a linker as disclosed herein. Optionally, any one of SEQ ID NOs: 4 to 50, or variants thereof are located between an extracellular domain as disclosed herein and an Fc domain as disclosed herein.

In embodiments, the present chimeric proteins may comprise variants of the joining linkers disclosed in Table 1, below. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NOs: 4 to 50.

In embodiments, the first and second joining linkers may be different or they may be the same.

Without wishing to be bound by theory, including a linker comprising at least a part of an Fc domain in a chimeric protein, helps avoid formation of insoluble and, likely, non-functional protein concatamers and/or aggregates. This is in part due to the presence of cysteines in the Fc domain which are capable of forming disulfide bonds between chimeric proteins.

In embodiments, a chimeric protein may comprise one or more joining linkers, as disclosed herein, and lack an Fc domain linker, as disclosed herein.

In embodiments, the first and/or second joining linkers are independently selected from the amino acid sequences of SEQ ID NOs: 4 to 50 and are provided in Table 1 below:

TABLE 1
Illustrative linkers (Fc domain linkers and joining linkers)

| SEQ | |
|---|---|
| ID NO. | Sequence |
| 1 | APEFLGGPSVFLFPPKPKDTLMISRTPEVTCVVVDVSQEDPEVQFNWYVD |
| | GVEVHNAKTKPREEQFNSTYRVVSVLTVLHQDWLSGKEYKCKVSSKGLPS |
| | SIEKTISNATGQPREPQVYTLPPSQEEMTKNQVSLTCLVKGFYPSDIAVEW |
| | ESNGQPENNYKTTPPVLDSDGSFFLYSRLTVDKSSWQEGNVFSCSVMHE |
| | ALHNHYTQKSLSLSLGK |
| 2 | APEFLGGPSVFLFPPKPKDQLMISRTPEVTCVVVDVSQEDPEVQFNWYVD |
| | GVEVHNAKTKPREEQFNSTYRVVSVLTTPHSDWLSGKEYKCKVSSKGLPS |
| | SIEKTISNATGQPREPQVYTLPPSQEEMTKNQVSLTCLVKGFYPSDIAVEW |
| | ESNGQPENNYKTTPPVLDSDGSFFLYSRLTVDKSSWQEGNVFSCSVLHEA |
| | LHNHYTQKSLSLSLGK |
| 3 | APEFLGGPSVFLFPPKPKDQLMISRTPEVTCVVVDVSQEDPEVQFNWYVD |
| | GVEVHNAKTKPREEQFNSTYRVVSVLTVLHQDWLSGKEYKCKVSSKGLPS |
| | SIEKTISNATGQPREPQVYTLPPSQEEMTKNQVSLTCLVKGFYPSDIAVEW |
| | ESNGQPENNYKTTPPVLDSDGSFFLYSRLTVDKSRWQEGNVFSCSVLHEA |
| | LHNHYTQKSLSLSLGK |
| 73 | EPKSCDKTHTCPPCPAPEAAGGPSVFLFPPKPKDTLMISRTPEVTCVVVDV |
| | SHEDPEVKFNWYVDGVEVHNAKTKPREEQYASTYRVVSVLTVLHQDWLN |
| | GKEYKCKVSNKALPAPIEKTISKAKGQPREPQVYTLPPSRDELTKNQVSLT |
| | CLVKGFYPSDIAVEWESNGQPENNYKTTPPVLDSDGSFFLYSKLTVDKSR |
| | WQQGNVFSCSVMHEALHNHYTQKSLSLSPGK |
| 4 | SKYGPPCPSCP |
| 5 | SKYGPPCPPCP |
| 6 | SKYGPP |
| 7 | IEGRMD |
| 8 | GGGVPRDCG |
| 9 | IEGRMDGGGGAGGGG |
| 10 | GGGSGGGS |
| 11 | GGGSGGGGSGGG |
| 12 | EGKSSGSGSESKST |
| 13 | GGSG |
| 14 | GGSGGGSGGGSG |
| 15 | EAAAKEAAAKEAAAK |
| 16 | EAAAREAAAREAAAREAAAR |
| 17 | GGGGSGGGGSGGGGSAS |
| 18 | GGGGAGGGG |
| 19 | GS or GGS or LE |
| 20 | GSGSGS |
| 21 | GSGSGSGSGS |
| 22 | GGGGSAS |
| 23 | APAPAPAPAPAPAPAPAPAP |
| 24 | CPPC |
| 25 | GGGGS |
| 26 | GGGGSGGGGS |
| 27 | GGGGSGGGGSGGGGS |
| 28 | GGGGSGGGGSGGGGSGGGGS |
| 29 | GGGGSGGGGSGGGGSGGGGSGGGGS |
| 30 | GGGGSGGGGSGGGGSGGGGSGGGGSGGGGS |
| 31 | GGGGSGGGGSGGGGSGGGGSGGGGGGGGSGGGGS |
| 32 | GGGGSGGGGSGGGGSGGGGSGGGGSGGGGSGGGGSGGGGS |
| 33 | GGSGGSGGGGSGGGGS |
| 34 | GGGGGGGG |
| 35 | GGGGGG |
| 36 | EAAAK |
| 37 | EAAAKEAAAK |
| 38 | EAAAKEAAAKEAAAK |
| 39 | AEAAAKEAAAKA |
| 40 | AEAAAKEAAAKEAAAKA |
| 41 | AEAAAKEAAAKEAAAKEAAAKA |
| 42 | AEAAAKEAAAKEAAAKEAAAKEAAAKA |
| 43 | AEAAAKEAAAKEAAAKEAAAKALEAEAAAKEAAAKEAAAKEAAAKA |
| 44 | PAPAP |
| 45 | KESGSVSSEQLAQFRSLD |
| 46 | GSAGSAAGSGEF |
| 47 | GGGSE |
| 48 | GSESG |
| 49 | GSEGS |
| 50 | GEGGSGEGSSGEGSSSEGGGSEGGGSEGGGSEGGS |

In embodiments, the joining linker substantially comprises glycine and serine residues (e.g., about 30%, or about 40%, or about 50%, or about 60%, or about 70%, or about 80%, or about 90%, or about 95%, or about 97%, or about 98%, or about 99%, or about 100% glycines and serines). For example, in embodiments, the joining linker is (Gly4Ser) n, where n is from about 1 to about 8, e.g., 1, 2, 3, 4, 5, 6, 7, or 8 (SEQ ID NO: 25 to SEQ ID NO: 32, respectively). In embodiments, the joining linker sequence is GGSGGSGGGGSGGGGS (SEQ ID NO: 33). Additional illustrative joining linkers include, but are not limited to, linkers having the sequence LE, (EAAAK)ₙ (n=1-3) (SEQ ID NO: 36 to SEQ ID NO: 38), A(EAAAK)ₙA(n=2-5) (SEQ ID NO: 39 to SEQ ID NO: 42), A(EAAAK)₄ALEA (EAAAK)₄A(SEQ ID NO: 43), PAPAP (SEQ ID NO: 44), 10 KESGSVSSEQLAQFRSLD (SEQ ID NO: 45), GSAGSAAGSGEF (SEQ ID NO: 46), and (XP) n, with X designating any amino acid, e.g., Ala, Lys, or Glu. In embodiments, the joining linker is GGS. In embodiments, a joining linker has the sequence (Gly)ₙ where n is any number from 1 to 100, for example: (Gly): (SEQ ID NO: 34) and (Gly) 6 (SEQ ID NO: 35).

In embodiments, the joining linker is one or more of GGGSE (SEQ ID NO: 47), GSESG (SEQ ID NO: 48), GSEGS (SEQ ID NO: 49), GEGGSGEGSSGEGSSSEGGGSEGGGSEGGGSEGGS (SEQ ID NO: 50), and a joining linker of randomly placed G, S, and E every 4 amino acid intervals.

The combination of a first joining linker, an Fc Domain linker, and a second joining linker is referend to herein as a “modular linker”. In embodiments, a heterologous chimeric protein comprises a modular linker as shown in Table 2:

TABLE 2
Illustrative modular linkers

| | | | Modular Linker = Joining |
|---|---|---|---|
| Joining | | Joining | Linker 1 + Fc + Joining |
| Linker 1 | Fc | Linker 2 | Linker 2 |
| SKYGPPCPSC | APEFLGGPSVFLFPPKPKDTL | IEGRMD | SKYGPPCPSCPAPEFLGGPSV |
| P | MISRTPEVTCVVVDVSQEDPE | (SEQ ID NO: | FLFPPKPKDTLMISRTPEVTCV |
| (SEQ ID NO: 4) | VQFNWYVDGVEVHNAKTKPR | 7) | WDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVMHEALHNHYTQK | | LTVDKSSWQEGNVFSCSVMH |
| | SLSLSLGK (SEQ ID NO: 1) | | EALHNHYTQKSLSLSLGKIEGR |
| | | | MD (SEQ ID NO: 51) |
| SKYGPPCPSC | APEFLGGPSVFLFPPKPKDQL | IEGRMD | SKYGPPCPSCPAPEFLGGPSV |
| P | MISRTPEVTCVVVDVSQEDPE | (SEQ ID NO: | FLFPPKPKDQLMISRTPEVTCV |
| (SEQ ID NO: 4) | VQFNWYVDGVEVHNAKTKPR | 7) | WDVSQEDPEVQFNWYVDGV |
| | EEQFNSTYRVVSVLTTPHSDW | | EVHNAKTKPREEQFNSTYRVV |
| | LSGKEYKCKVSSKGLPSSIEKT | | SVLTTPHSDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVLHEALHNHYTQKS | | LTVDKSSWQEGNVFSCSVLHE |
| | LSLSLGK (SEQ ID NO: 2) | | ALHNHYTQKSLSLSLGKIEGR |
| | | | MD (SEQ ID NO: 52) |
| SKYGPPCPSC | APEFLGGPSVFLFPPKPKDQL | IEGRMD | SKYGPPCPSCPAPEFLGGPSV |
| P | MISRTPEVTCVVVDVSQEDPE | (SEQ ID NO: | FLFPPKPKDQLMISRTPEVTCV |
| (SEQ ID NO: 4) | VQFNWYVDGVEVHNAKTKPR | 7) | WDVSQEDPEVQFNWYVDGV |
| | EEQFNSTYRVVSVLTVLHQDW | | EVHNAKTKPREEQFNSTYRVV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSRWQE | | NNYKTTPPVLDSDGSFFLYSR |
| | GNVFSCSVLHEALHNHYTQKS | | LTVDKSRWQEGNVFSCSVLHE |
| | LSLSLGK (SEQ ID NO: 3) | | ALHNHYTQKSLSLSLGKIEGR |
| | | | MD (SEQ ID NO: 53) |
| SKYGPPCPPC | APEFLGGPSVFLFPPKPKDTL | IEGRMD | SKYGPPCPPCPAPEFLGGPSV |
| P | MISRTPEVTCVVVDVSQEDPE | (SEQ ID NO: | FLFPPKPKDTLMISRTPEVTCV |
| (SEQ ID NO: 5) | VQFNWYVDGVEVHNAKTKPR | 7) | WDVSQEDPEVQFNWYVDGV |
| | EEQFNSTYRVVSVLTVLHQDW | | EVHNAKTKPREEQFNSTYRWV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVMHEALHNHYTQK | | LTVDKSSWQEGNVFSCSVMH |
| | SLSLSLGK (SEQ ID NO: 1) | | EALHNHYTQKSLSLSLGKIEGR |
| | | | MD (SEQ ID NO: 54) |
| SKYGPPCPPC | APEFLGGPSVFLFPPKPKDQL | IEGRMD | SKYGPPCPPCPAPEFLGGPSV |
| P | MISRTPEVTCVVVDVSQEDPE | (SEQ ID NO: | FLFPPKPKDQLMISRTPEVTCV |
| (SEQ ID NO: 5) | VQFNWYVDGVEVHNAKTKPR | 7) | WVDVSQEDPEVQFNWYVDGV |
| | EEQFNSTYRVVSVLTTPHSDW | | EVHNAKTKPREEQFNSTYRWV |
| | LSGKEYKCKVSSKGLPSSIEKT | | SVLTTPHSDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSSWQE | | NNYKTTPPVLDSDGSFFLYSR |
| | GNVFSCSVLHEALHNHYTQKS | | LTVDKSSWQEGNVFSCSVLHE |
| | LSLSLGK (SEQ ID NO: 2) | | ALHNHYTQKSLSLSLGKIEGR |
| | | | MD (SEQ ID NO: 55) |
| SKYGPPCPPC | APEFLGGPSVFLFPPKPKDQL | IEGRMD | SKYGPPCPPCPAPEFLGGPSV |
| P | MISRTPEVTCVVVDVSQEDPE | (SEQ ID NO: | FLFPPKPKDQLMISRTPEVTCV |
| (SEQ ID NO: 5) | VQFNWYVDGVEVHNAKTKPR | 7) | VVDVSQEDPEVQFNWYVDGV |
| | EEQFNSTYRVVSVLTVLHQDW | | EVHNAKTKPREEQFNSTYRWV |
| | LSGKEYKCKVSSKGLPSSIEKT | | SVLTVLHQDWLSGKEYKCKVS |
| | ISNATGQPREPQVYTLPPSQE | | SKGLPSSIEKTISNATGQPREP |
| | EMTKNQVSLTCLVKGFYPSDIA | | QVYTLPPSQEEMTKNQVSLTC |
| | VEWESNGQPENNYKTTPPVL | | LVKGFYPSDIAVEWESNGQPE |
| | DSDGSFFLYSRLTVDKSRWQE | | NNYKTTPPVLDSDGSFFLYSR |
| | GNVFSCSVLHEALHNHYTQKS | | LTVDKSRWQEGNVFSCSVLHE |
| | LSLSLGK (SEQ ID NO: 3) | | ALHNHYTQKSLSLSLGKIEGR |
| | | | MD (SEQ ID NO: 56) |

In embodiments, the present heterologous chimeric proteins may comprise variants of the modular linkers disclosed in Table 2, above. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NOs: 51 to 56.

In embodiments, the linker may be flexible, including without limitation highly flexible. In embodiments, the linker may be rigid, including without limitation a rigid alpha helix. Characteristics of illustrative joining linkers is shown below in Table 3:

TABLE 3
Characteristics of illustrative joining linkers

| Joining Linker Sequence | Characteristics |
|---|---|
| SKYGPPCPPCP (SEQ ID NO: 5) | lgG4 Hinge Region |
| IEGRMD (SEQ ID NO: 7) | Linker |
| GGGVPRDCG (SEQ ID NO: 8) | Flexible |
| GGGSGGGS (SEQ ID NO: 10) | Flexible |
| GGGSGGGGSGGG (SEQ ID NO: 11) | Flexible |
| EGKSSGSGSESKST (SEQ ID NO: 12) | Flexible + soluble |
| GGSG (SEQ ID NO: 13) | Flexible |
| GGSGGGSGGGSG (SEQ ID NO: 14) | Flexible |
| EAAAKEAAAKEAAAK (SEQ ID NO: 15) | Rigid Alpha Helix |
| EAAAREAAAREAAAREAAAR (SEQ ID NO: 16) | Rigid Alpha Helix |
| GGGGSGGGGSGGGGSAS (SEQ ID NO: 17) | Flexible |
| GGGGAGGGG (SEQ ID NO: 18) | Flexible |
| GS (SEQ ID NO: 19) | Highly flexible |
| GSGSGS (SEQ ID NO: 20) | Highly flexible |
| GSGSGSGSGS (SEQ ID NO: 21) | Highly flexible |
| GGGGSAS (SEQ ID NO: 22) | Flexible |
| APAPAPAPAPAPAPAPAPAP (SEQ ID NO: 23) | Rigid |

In embodiments, the linker may be functional. For example, without limitation, the linker may function to improve the folding and/or stability, improve the expression, improve the pharmacokinetics, and/or improve the bioactivity of the present heterologous chimeric protein. In another example, the linker may function to target the heterologous chimeric protein to a particular cell type or location.

In embodiments, a heterologous chimeric protein comprises only one joining linkers.

In embodiments, a heterologous chimeric protein lacks joining linkers.

In embodiments, the linker is a synthetic linker such as polyethylene glycol (PEG).

In embodiments, a heterologous chimeric protein has a first domain which is sterically capable of binding its ligand/receptor and/or the second domain which is sterically capable of binding its ligand/receptor. Thus, there is enough overall flexibility in the chimeric protein and/or physical distance between an extracellular domain (or portion thereof) and the rest of the chimeric protein such that the ligand/receptor binding domain of the extracellular domain is not sterically hindered from binding its ligand/receptor. This flexibility and/or physical distance (which is referred to as “slack”) may be normally present in the extracellular domain(s), normally present in the linker, and/or normally present in the chimeric protein (as a whole). Alternately, or additionally, an amino acid sequence (for example) may be added to one or more extracellular domains and/or to the linker to provide the slack needed to avoid steric hindrance. Any amino acid sequence that provides slack may be added. In embodiments, the added amino acid sequence comprises the sequence (Gly)ₙ where n is any number from 1 to 100. Additional examples of addable amino acid sequence include the joining linkers described in Table 1 and Table 3. In embodiments, a polyethylene glycol (PEG) linker may be added between an extracellular domain and a linker to provide the slack needed to avoid steric hindrance. Such PEG linkers are well known in the art.

In one aspect, the present disclosure provides a chimeric protein having a general structure of: N terminus-(a)-(b)-(c)-C terminus, or a nucleic acid encoding the same, wherein: (a) is a first domain comprising a portion of TNF receptor (TNFR2) that is capable of binding TNFa and/or capable of oligomerizing with a cellular TNF receptor; (b) is a linker adjoining the first domain and a second domain, optionally comprising a hinge-C_{H2}-C_{H3} Fc domain, and (c) is a second domain comprising a portion of a C-type lectin receptor (CLR) capable of binding a ligand.

In embodiments, the first domain comprises a portion of TNFR2 comprises the extracellular domain of TNFR2, or a fragment thereof. In embodiments, the portion of TNFR2 comprises an amino acid sequence that is at least about 90%, or at least about 95%, or at least about 97%, or at least about 98%, or at least about 99% identical to the amino acid sequence of SEQ ID NO: 57, and capable of binding TNFa and/or capable of oligomerizing with a cellular TNF receptor. In embodiments, the portion of CLR comprises the extracellular domain of CLR, or a fragment thereof that is capable of binding a natural ligand of the CLR. In embodiments, the portion of CLR comprises an amino acid sequence that is at least about 90%, or at least about 95%, or at least about 97%, or at least about 98%, or at least about 99% identical to an amino acid sequence selected from SEQ ID NO: 58-65. In embodiments, the hinge-C_{H2}-C_{H3} Fc domain is derived from IgG1 or IgG4, e.g., human IgG1 or IgG4. In embodiments, the hinge-C_{H2}-C_{H3} Fc domain comprises an amino acid sequence that is at least about 95% identical to the amino acid sequence of SEQ ID NO: 1, SEQ ID NO: 2, SEQ ID NO: 3, or SEQ ID NO: 73. In embodiments, the linker further comprises the linker comprises one or more joining linkers, such joining linkers independently selected from SEQ ID NOs: 4 to 50. In embodiments, the linker comprises two or more joining linkers each joining linker independently selected from SEQ ID NOs: 4 to 50; wherein one joining linker is N terminal to the hinge-C_{H2}-C_{H3}-Fc domain and another joining linker is C terminal to the hinge-C_{H2}-C_{H3}-Fc domain.

In embodiments, where the chimeric protein comprises a portion of TNFR2, a joining linker preceding an Fc domain, the Fc domain, a joining linker following the Fc domain, and a portion Clec7a, the chimeric protein may comprise the following structure:

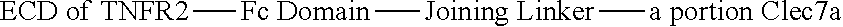

In embodiments, the chimeric protein comprises: an extracellular domain of TNFR2 comprising an amino acid sequence that is at least about 90%, or at least about 95%, or at least about 97%, or at least about 98%, or at least about 99% identical to the amino acid sequence of SEQ ID NO: 57; a portion of Clec7a capable of binding a beta-1,3-linked and/or beta-1,6-linked glucan, and comprising an amino acid sequence that is at least about 90%, or at least about 95%, or at least about 97%, or at least about 98%, or at least about 99% identical to the amino acid sequence of SEQ ID NO: 58 or SEQ ID NO: 59; and a linker adjoining the extracellular domains. In embodiments, the hinge-C_{H2}-C_{H3} Fc domain is derived from IgG1 or IgG4, e.g., human IgG1 or IgG4. In embodiments, the hinge-C_{H2}-C_{H3} Fc domain comprises an amino acid sequence that is at least about 95% identical to the amino acid sequence of SEQ ID NO: 1, SEQ ID NO: 2, SEQ ID NO: 3, or SEQ ID NO: 73. In embodiments, the linker further comprises the linker comprises one or more joining linkers, such joining linkers independently selected from SEQ ID NOs: 4 to 50. In embodiments, the linker comprises two or more joining linkers each joining linker independently selected from SEQ ID NOs: 4 to 50; wherein one joining linker is N terminal to the hinge-C_{H2}-C_{H3}-Fc domain and another joining linker is C terminal to the hinge-C_{H2}-C_{H3}-Fc domain.

In one aspect, the present disclosure provides a chimeric protein having a general structure of: N terminus-(a)-(b)-(c)-C terminus, or a nucleic acid encoding the same, wherein: (a) is a first domain comprising a portion of the extracellular domain of TNF receptor (TNFR2) that is capable of binding TNFa and/or capable of oligomerizing with a cellular TNF receptor; (b) is a linker adjoining the first domain and a second domain comprising a hinge-C_{H2}-C_{H3} Fc domain, and (c) is the second domain comprising a portion of the extracellular domain of C-Type Lectin Domain Containing 7A(Clec7A) capable of binding a beta-1,3-linked and/or beta-1,6-linked glucan.

In one aspect, the present disclosure provides a chimeric protein having a general structure of: N terminus-(a)-(b)-(c)-C terminus, or a nucleic acid encoding the same, wherein: (a) is a first domain comprising a portion of the extracellular domain of TNF receptor (TNFR2) capable of binding TNFa and/or capable of oligomerizing with a cellular TNF receptor, and comprising an amino acid sequence that is at least about 95% identical to the amino acid sequence of SEQ ID NO: 57; (b) is a linker adjoining the first domain and a second domain comprising a hinge-C_{H2}-C_{H3} Fc domain comprising an amino acid sequence that is at least about 95% identical to the amino acid sequence selected from SEQ ID NO: 1, SEQ ID NO: 2, SEQ ID NO: 3, and SEQ ID NO: 73, and (c) is the second domain comprising a portion of the C-type lectin binding domain (CLD) of C-Type Lectin Domain Containing 7A(Clec7A) capable of binding a ligand comprising a beta-1,3-linked and/or beta-1,6-linked glucan, and comprising an amino acid sequence that is at least about 95% identical to the amino acid sequence of SEQ ID NO: 58 or SEQ ID NO: 59.

An illustrative TNFR2-Fc-Clec7a chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the extracellular domain of Clec7a is shown in an italics font):

(SEQ ID NO: 66)
LPAQVAFTPYAPEPGSTCRLREYYDQTAQMCCSKCSPGQHAKVFCTKTSDTVCDSCEDSTYTQL
WNWVPECLSCGSRCSSDQVETQACTREQNRICTCRPGWYCALSKQEGCRLCAPLRKCRPGFGV
ARPGTETSDVVCKPCAPGTFSNTTSSTDICRPHQICNVVAIPGNASMDAVCTSTSPTRSMAPGAV
HLPQPVSTRSQHTQPTPEPSTAPSTSFLLPMGPSPPAEGSTGDEPKSCDKTHTCPPCPAPEAAG
GPSVFLFPPKPKDTLMISRTPEVTCVVVDVSHEDPEVKFNWYVDGVEVHNAKTKPREEQYASTY
RVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSV
MHEALHNHYTQKSLSLSPGKIEGRMDTMAIWRSNSGSNTLENGYFLSRNKENHSQPTQSSLEDS
VTPTKAVKTTGVLSSPCPPNWIIYEKSCYLFSMSLNSWDGSKRQCWQLGSNLLKIDSSNELGFIVK
QVSSQPDNSFWIGLSRPQTEVPWLWEDGSTFSSNLFQIRTTATQENPSPNCVWIHVSVIYDQLCS
VPSYSICEKKFSM.

In embodiments, the chimeric protein comprises a variant of the TNFR2-Fc-Clec7a chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 66.

An illustrative TNFR2-Fc-Clec7a chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the C-type lectin binding domain (CLD) of Clec7a is shown in an italics font):

(SEQ ID NO: 67)
LPAQVAFTPYAPEPGSTCRLREYYDQTAQMCCSKCSPGQHAKVFCTKTSDTVCDSCEDSTYTQL
WNWVPECLSCGSRCSSDQVETQACTREQNRICTCRPGWYCALSKQEGCRLCAPLRKCRPGFGV
ARPGTETSDWVCKPCAPGTFSNTTSSTDICRPHQICNVVAIPGNASMDAVCTSTSPTRSMAPGAV
HLPQPVSTRSQHTQPTPEPSTAPSTSFLLPMGPSPPAEGSTGDEPKSCDKTHTCPPCPAPEAAG
GPSVFLFPPKPKDTLMISRTPEVTCVVVDVSHEDPEVKFNWYVDGVEVHNAKTKPREEQYASTY
RVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSV
MHEALHNHYTQKSLSLSPGKIEGRMDSSPCPPNWIIYEKSCYLFSMSLNSWDGSKRQCWQLGS
NLLKIDSSNELGFIVKQVSSQPDNSFWIGLSRPQTEVPWLWEDGSTFSSNLFQIRTTATQENPSPN
CVWIHVSVIYDQLCSVPSYSICEKKFSM.

In embodiments, the chimeric protein comprises a variant of the TNFR2-Fc-Clec7a chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 67.

An illustrative mouse TNFR2-Fc-Clec7a chimeric protein has the following sequence (the extracellular domain of mouse TNFR2 is shown by an underline, a linker comprising a mutant Fc domain of mouse IgG1 is shown in boldface font, a joining linker is shown in an underlined-boldface-italic font, and the extracellular of Clec7a is shown in an italics font):

(SEQ ID NO: 81)
VPAQVVLTPYKPEPGYECQISQEYYDRKAQMCCAKCPPGQYVKHFCNKTSDTVCADCEASMYT
QVWNQFRTCLSCSSSCTTDQVEIRACTKQQNRVCACEAGRYCALKTHSGSCRQCMRLSKCGP
GFGVASSRAPNGNVLCKACAPGTFSDTTSSTDVCRPHRICSILAIPGNASTDAVCAPESPTLSAIP
RTLYVSQPEPTRSQPLDQEPGPSQTPSILTSLGSTPIIEQSTKGGVPRDCGCKPCICTVPEVSSVF
IFPPKPKDVLTITLTPKVTCVVVDISKDDPEVQFSWFVDDVEVHTAQTQPREEQFNSTFRSVSEL
PIMHQDWLNGKEFKCRVNSAAFPAPIEKTISKTKGRPKAPQVYTIPPPKEQMAKDKVSLTCMIT
DFFPEDITVEWQWNGQPAENYKNTQPIMDTDGSYFVYSKLNVQKSNWEAGNTFTCSVLHEGLH
NHHTEKSLSHSPGKIEGRMDGHNSGRNPEEKDNFLSRNKENHKPTESSLDEKVAPSKASQTTG
GFSQPCLPNWIMHGKSCYLFSFSGNSWYGSKRHCSQLGAHLLKIDNSKEFEFIESQTSSHRINAF
WIGLSRNQSEGPWFWEDGSAFFPNSFQVRNTAPQESLLHNCVWIHGSEVYNQICNTSSYSICEK
EL.

In embodiments, the chimeric protein comprises a variant of the mouse TNFR2-Fc-Clec7a chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 81.

In embodiments, where the chimeric protein comprises an extracellular domain (ECD) of TNFR2, a joining linker preceding an Fc domain, the Fc domain, a joining linker following the Fc domain, and a portion of langerin, the chimeric protein may comprise the following structure:

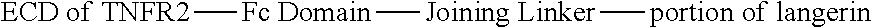

In embodiments, the chimeric protein comprises: an extracellular domain of TNFR2 comprising an amino acid sequence that is at least about 90%, or at least about 95%, or at least about 97%, or at least about 98%, or at least about 99% identical to the amino acid sequence of SEQ ID NO: 57; a portion of langerin capable of binding a ligand comprising a sulfated glycan, a mannosylated glycan, a keratan sulfate (KS) and/or a beta-glucan, and comprising an amino acid sequence that is at least about 90%, or at least about 95%, or at least about 97%, or at least about 98%, or at least about 99% identical to the amino acid sequence of SEQ ID NO: 60 or SEQ ID NO: 61; and a linker adjoining the extracellular domains. In embodiments, the hinge-C_{H2}-C_{H3} Fc domain is derived from IgG1 or IgG4, e.g., human IgG1 or IgG4. In embodiments, the hinge-C_{H2}-C_{H3} Fc domain comprises an amino acid sequence that is at least about 95% identical to the amino acid sequence of SEQ ID NO: 1, SEQ ID NO: 2, SEQ ID NO: 3 or SEQ ID NO: 73. In embodiments, the linker further comprises the linker comprises one or more joining linkers, such joining linkers independently selected from SEQ ID NOs: 4 to 50. In embodiments, the linker comprises two or more joining linkers each joining linker independently selected from SEQ ID NOs: 4 to 50; wherein one joining linker is N terminal to the hinge-C_{H2}-C_{H3}-Fc domain and another joining linker is C terminal to the hinge-C_{H2}-C_{H3}-Fc domain.

In one aspect, the present disclosure provides a chimeric protein having a general structure of: N terminus-(a)-(b)-(c)-C terminus, or a nucleic acid encoding the same, wherein: (a) is a first domain comprising a portion of the extracellular domain of TNF receptor (TNFR2) that is capable of binding TNFa and/or capable of oligomerizing with a cellular TNF receptor; (b) is a linker adjoining the first domain and a second domain comprising a hinge-C_{H2}-C_{H3} Fc domain, and (c) is the second domain comprising a portion of the extracellular domain of langerin capable of binding a sulfated glycan, a mannosylated glycan, a keratan sulfate (KS) and/or a beta-glucan.

In one aspect, the present disclosure provides a chimeric protein having a general structure of: N terminus-(a)-(b)-(c)-C terminus, or a nucleic acid encoding the same, wherein: (a) is a first domain comprising a portion of the extracellular domain of TNF receptor (TNFR2) capable of binding TNFa and/or capable of oligomerizing with a cellular TNF receptor, and comprising an amino acid sequence that is at least about 95% identical to the amino acid sequence of SEQ ID NO: 57; (b) is a linker adjoining the first domain and a second domain comprising a hinge-C_{H2}-C_{H3} Fc domain comprising an amino acid sequence that is at least about 95% identical to the amino acid sequence selected from SEQ ID NO: 1, SEQ ID NO: 2, SEQ ID NO: 3, and SEQ ID NO: 73, and (c) is the second domain comprising a portion of the extracellular domain of langerin capable of binding a ligand comprising a sulfated glycan, a mannosylated glycan, a keratan sulfate (KS) and/or a beta-glucan, and comprising an amino acid sequence that is at least about 95% identical to the amino acid sequence of SEQ ID NO: 60 or SEQ ID NO: 61.

An illustrative TNFR2-Fc-langerin chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the extracellular domain of langerin is shown in an italics font):

(SEQ ID NO: 68)
LPAQVAFTPYAPEPGSTCRLREYYDQTAQMCCSKCSPGQHAKVFCTKTSDTVCDSCEDSTYTQL
WNWVPECLSCGSRCSSDQVETQACTREQNRICTCRPGWYCALSKQEGCRLCAPLRKCRPGFGV
ARPGTETSDWVCKPCAPGTFSNTTSSTDICRPHQICNVVAIPGNASMDAVCTSTSPTRSMAPGAV
HLPQPVSTRSQHTQPTPEPSTAPSTSFLLPMGPSPPAEGSTGDEPKSCDKTHTCPPCPAPEAAG
GPSVFLFPPKPKDTLMISRTPEVTCVVVDVSHEDPEVKFNWYVDGVEVHNAKTKPREEQYASTY
RVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSV
MHEALHNHYTQKSLSLSPGKIEGRMDPRFMGTISDVKTNVQLLKGRVDNISTLDSEIKKNSDGME
AAGVQIQMVNESLGYVRSQFLKLKTSVEKANAQIQILTRSWEEVSTLNAQIPELKSDLEKASALNTK
IRALQGSLENMSKLLKRQNDILQVVSQGWKYFKGNFYYFSLIPKTWYSAEQFCVSRNSHLTSVTSE
SEQEFLYKTAGGLIYWIGLTKAGMEGDWSWVDDTPFNKVQSVRFWIPGEPNNAGNNEHCGNIKA
PSLQAWNDAPCDKTFLFICKRPYVPSEP.

In embodiments, the chimeric protein comprises a variant of the TNFR2-Fc-langerin chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 68.

An illustrative TNFR2-Fc-langerin chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the C-type lectin binding domain (CLD) of langerin is shown in an italics font):

(SEQ ID NO: 69)
LPAQVAFTPYAPEPGSTCRLREYYDQTAQMCCSKCSPGQHAKVFCTKTSDTVCDSCEDSTYTQL
WNWVPECLSCGSRCSSDQVETQACTREQNRICTCRPGWYCALSKQEGCRLCAPLRKCRPGFGV
ARPGTETSDVVCKPCAPGTFSNTTSSTDICRPHQICNVVAIPGNASMDAVCTSTSPTRSMAPGAV
HLPQPVSTRSQHTQPTPEPSTAPSTSFLLPMGPSPPAEGSTGDEPKSCDKTHTCPPCPAPEAAG
GPSVFLFPPKPKDTLMISRTPEVTCVVVDVSHEDPEVKFNWYVDGVEVHNAKTKPREEQYASTY
RVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSV
MHEALHNHYTQKSLSLSPGKIEGRMDQVVSQGWKYFKGNFYYFSLIPKTWYSAEQFCVSRNSHL
TSVTSESEQEFLYKTAGGLIYWIGLTKAGMEGDWSWVDDTPFNKVQSVRFWIPGEPNNAGNNEH
CGNIKAPSLQAWNDAPCDKTFLFICKRPYVPSEP.

In embodiments, the chimeric protein comprises a variant of the TNFR2-Fc-langerin chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 69.

An illustrative mouse TNFR2-Fc-Langerin chimeric protein has the following sequence (the extracellular domain of mouse TNFR2 is shown by an underline, a linker comprising a mutant Fc domain of mouse IgG1 is shown in boldface font, a joining linker is shown in an underlined-boldface-italic font, and the extracellular of Langerin is shown in an italics font):

(SEQ ID NO: 84)
VPAQVVLTPYKPEPGYECQISQEYYDRKAQMCCAKCPPGQYVKHFCNKTSDTVCADCEASMYTQ
VWNQFRTCLSCSSSCTTDQVEIRACTKQQNRVCACEAGRYCALKTHSGSCRQCMRLSKCGPGE
GVASSRAPNGNVLCKACAPGTFSDTTSSTDVCRPHRICSILAIPGNASTDAVCAPESPTLSAIPRTL
YVSQPEPTRSQPLDQEPGPSQTPSILTSLGSTPIIEQSTKGGVPRDCGCKPCICTVPEVSSVFIFPP
KPKDVLTITLTPKVTCVVVDISKDDPEVQFSWFVDDVEVHTAQTQPREEQFNSTFRSVSELPIMH
QDWLNGKEFKCRVNSAAFPAPIEKTISKTKGRPKAPQVYTIPPPKEQMAKDKVSLTCMITDFFPE
DITVEWQWNGQPAENYKNTQPIMDTDGSYFVYSKLNVQKSNWEAGNTFTCSVLHEGLHNHHTE
KSLSHSPGKIEGRMDQAVFYPRLMGKILDVKSDAQMLKGRVDNISTLGSDLKTERGRVDDAEVQ
MQIVNTTLKRVRSQILSLETSMKIANDQLQILTMSWGEVDSLSAKIPELKRDLDKASALNTKVQGLQ
NSLENVNKLLKQQSDILEMVARGWKYFSGNFYYFSRTPKTWYSAEQFCISRKAHLTSVSSESEQK
FLYKAADGIPHWIGLTKAGSEGDWYWVDQTSFNKEQSRRFWIPGEPNNAGNNEHCANIRVSALK
CWNDGPCDNTFLFICKRPYVQTTE.

In embodiments, the chimeric protein comprises a variant of the mouse TNFR2-Fc-Langerin chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 97%, or at least about 95%, or at least about 96%, or at least about 97%, or at least about 98%, or at least about 99% sequence identity with SEQ ID NO: 84.

In embodiments, where the chimeric protein comprises an extracellular domain (ECD) of TNFR2, a joining linker preceding an Fc domain, the Fc domain, a joining linker following the Fc domain, and a portion of DC-SIGN the chimeric protein may comprise the following structure:

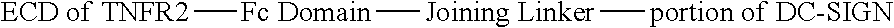

In embodiments, the chimeric protein comprises: an extracellular domain of TNFR2 comprising an amino acid sequence that is at least about 90%, or at least about 95%, or at least about 97%, or at least about 98%, or at least about 99% identical to the amino acid sequence of SEQ ID NO: 57; a portion of DC-SIGN comprising an amino acid sequence that is at least about 90%, or at least about 95%, or at least about 97%, or at least about 98%, or at least about 99% identical to the amino acid sequence of SEQ ID NO: 62 or SEQ ID NO: 63; and a linker adjoining the extracellular domains. In embodiments, the hinge-C_{H2}-C_{H3} Fc domain is derived from IgG1 or IgG4, e.g., human IgG1 or IgG4. In embodiments, the hinge-C_{H2}-C_{H3} Fc domain comprises an amino acid sequence that is at least about 95% identical to the amino acid sequence of SEQ ID NO: 1, SEQ ID NO: 2, SEQ ID NO: 3, and SEQ ID NO: 73. In embodiments, the linker further comprises the linker comprises one or more joining linkers, such joining linkers independently selected from SEQ ID NOs: 4 to 50. In embodiments, the linker comprises two or more joining linkers each joining linker independently selected from SEQ ID NOs: 4 to 50; wherein one joining linker is N terminal to the hinge-C_{H2}-C_{H3}-Fc domain and another joining linker is C terminal to the hinge-C_{H2}-C_{H3}-Fc domain.

In one aspect, the present disclosure provides a chimeric protein having a general structure of: N terminus-(a)-(b)-(c)-C terminus, or a nucleic acid encoding the same, wherein: (a) is a first domain comprising a portion of the extracellular domain of TNF receptor (TNFR2) that is capable of binding TNFa and/or capable of oligomerizing with a cellular TNF receptor; (b) is a linker adjoining the first domain and a second domain comprising a hinge-C_{H2}-C_{H3} Fc domain, and (c) is the second domain comprising a portion of the extracellular domain of DC-SIGN capable of binding an Intercellular Adhesion Molecule 2 (ICAM2) and/or Intercellular Adhesion Molecule 3 (ICAM3).

In one aspect, the present disclosure provides a chimeric protein having a general structure of: N terminus-(a)-(b)-(c)-C terminus, or a nucleic acid encoding the same, wherein: (a) is a first domain comprising a portion of the extracellular domain of TNF receptor (TNFR2) capable of binding TNFa and/or capable of oligomerizing with a cellular TNF receptor, and comprising an amino acid sequence that is at least about 95% identical to the amino acid sequence of SEQ ID NO: 57; (b) is a linker adjoining the first domain and a second domain comprising a hinge-C_{H2}-C_{H3} Fc domain comprising an amino acid sequence that is at least about 95% identical to the amino acid sequence selected from SEQ ID NO: 1, SEQ ID NO: 2, SEQ ID NO: 3, and SEQ ID NO: 73, and (c) is the second domain comprising a portion of the extracellular domain of DC-SIGN capable of binding an Intercellular Adhesion Molecule 2 (ICAM2) and/or Intercellular Adhesion Molecule 3 (ICAM3, and comprising an amino acid sequence that is at least about 95% identical to the amino acid sequence of SEQ ID NO: 62 or SEQ ID NO: 63.

An illustrative TNFR2-Fc-DC-SIGN chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the extracellular domain of DC-SIGN is shown in an italics font):

(SEQ ID NO: 70)
LPAQVAFTPYAPEPGSTCRLREYYDQTAQMCCSKCSPGQHAKVFCTKTSDTVCDSCEDSTYTQL
WNWVPECLSCGSRCSSDQVETQACTREQNRICTCRPGWYCALSKQEGCRLCAPLRKCRPGFGV
ARPGTETSDVVCKPCAPGTFSNTTSSTDICRPHQICNVVAIPGNASMDAVCTSTSPTRSMAPGAV
HLPQPVSTRSQHTQPTPEPSTAPSTSFLLPMGPSPPAEGSTGDEPKSCDKTHTCPPCPAPEAAG
GPSVFLFPPKPKDTLMISRTPEVTCVVVDVSHEDPEVKFNWYVDGVEVHNAKTKPREEQYASTY
RVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSV
MHEALHNHYTQKSLSLSPGKIEGRMDQVSKVPSSISQEQSRQDAIYQNLTQLKAAVGELSEKSKL
QEIYQELTQLKAAVGELPEKSKLQEIYQELTRLKAAVGELPEKSKLQEIYQELTWLKAAVGELPEKS
KMQEIYQELTRLKAAVGELPEKSKQQEIYQELTRLKAAVGELPEKSKQQEIYQELTRLKAAVGELP
EKSKQQEIYQELTQLKAAVERLCHPCPWEWTFFQGNCYFMSNSQRNWHDSITACKEVGAQLVVI
KSAEEQNFLQLQSSRSNRFTWMGLSDLNQEGTWQWVDGSPLLPSFKQYWNRGEPNNVGEEDC
AEFSGNGWNDDKCNLAKFWICKKSAASCSRDEEQFLSPAPATPNPPPA.

In embodiments, the chimeric protein comprises a variant of the TNFR2-Fc-DC-SIGN chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 70.

An illustrative TNFR2-Fc-DC-SIGN chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the C-type lectin binding domain (CLD) of DC-SIGN is shown in an italics font):

(SEQ ID NO: 71)
LPAQVAFTPYAPEPGSTCRLREYYDQTAQMCCSKCSPGQHAKVFCTKTSDTVCDSCEDSTYTQL
WNWVPECLSCGSRCSSDQVETQACTREQNRICTCRPGWYCALSKQEGCRLCAPLRKCRPGFGV
ARPGTETSDVVCKPCAPGTFSNTTSSTDICRPHQICNVVAIPGNASMDAVCTSTSPTRSMAPGAV
HLPQPVSTRSQHTQPTPEPSTAPSTSFLLPMGPSPPAEGSTGDEPKSCDKTHTCPPCPAPEAAG
GPSVFLFPPKPKDTLMISRTPEVTCVVVDVSHEDPEVKFNWYVDGVEVHNAKTKPREEQYASTY
RVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSV
MHEALHNHYTQKSLSLSPGKIEGRMDHPCPWEWTFFQGNCYFMSNSQRNWHDSITACKEVGA
QLVVIKSAEEQNFLQLQSSRSNRFTWMGLSDLNQEGTWQWVDGSPLLPSFKQYWNRGEPNNVG
EEDCAEFSGNGWNDDKCNLAKFWICK.

In embodiments, the chimeric protein comprises a variant of the TNFR2-Fc-DC-SIGN chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 71.

An illustrative mouse TNFR2-Fc-DC-SIGN chimeric protein has the following sequence (the extracellular domain of mouse TNFR2 is shown by an underline, a linker comprising a mutant Fc domain of mouse IgG1 is shown in boldface font, a joining linker is shown in an underlined-boldface-italic font, and the extracellular of DC-SIGN is shown in an italics font):

(SEQ ID NO: 82)
VPAQVVLTPYKPEPGYECQISQEYYDRKAQMCCAKCPPGQYVKHFCNKTSDTVCADCEASMYTQ
VWNQFRTCLSCSSSCTTDQVEIRACTKQQNRVCACEAGRYCALKTHSGSCRQCMRLSKCGPGF
GVASSRAPNGNVLCKACAPGTFSDTTSSTDVCRPHRICSILAIPGNASTDAVCAPESPTLSAIPRTL
YVSQPEPTRSQPLDQEPGPSQTPSILTSLGSTPIIEQSTKGGVPRDCGCKPCICTVPEVSSVFIFPP
KPKDVLTITLTPKVTCVVVDISKDDPEVQFSWFVDDVEVHTAQTQPREEQFNSTFRSVSELPIMH
QDWLNGKEFKCRVNSAAFPAPIEKTISKTKGRPKAPQVYTIPPPKEQMAKDKVSLTCMITDFFPE
DITVEWQWNGQPAENYKNTQPIMDTDGSYFVYSKLNVQKSNWEAGNTFTCSVLHEGLHNHHTE
KSLSHSPGKIEGRMDVKVYKIPSSQEENNQMNVYQELTQLKAGVDRLCRSCPWDWTHFQGSCY
FFSVAQKSWNDSATACHNVGAQLVVIKSDEEQNFLQQTSKKRGYTWMGLIDMSKESTWYWVDG
SPLTLSFMKYWSKGEPNNLGEEDCAEFRDDGWNDTKCTNKKFWICKKLSTSCPSK.

In embodiments, the chimeric protein comprises a variant of the mouse TNFR2-Fc-DC-SIGN chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 82.

In embodiments, where the chimeric protein comprises an extracellular domain (ECD) of TNFR2, a joining linker preceding an Fc domain, the Fc domain, a joining linker following the Fc domain, and a portion of Dectin-2, the chimeric protein may comprise the following structure:

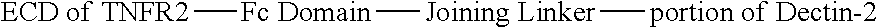

In embodiments, the chimeric protein comprises: an extracellular domain of TNFR2 comprising an amino acid sequence that is at least about 90%, or at least about 95%, or at least about 97%, or at least about 98%, or at least about 99% identical to the amino acid sequence of SEQ ID NO: 57; a portion of Dectin-2 comprising an amino acid sequence that is at least about 90%, or at least about 95%, or at least about 97%, or at least about 98%, or at least about 99% identical to the amino acid sequence of SEQ ID NO: 64 or SEQ ID NO: 65; and a linker adjoining the extracellular domains. In embodiments, the hinge-C_{H2}-C_{H3} Fc domain is derived from IgG1 or IgG4, e.g., human IgG1 or IgG4. In embodiments, the hinge-C_{H2}-C_{H3} Fc domain comprises an amino acid sequence that is at least about 95% identical to the amino acid sequence of SEQ ID NO: 1, SEQ ID NO: 2, SEQ ID NO: 3, or SEQ ID NO: 73. In embodiments, the linker further comprises the linker comprises one or more joining linkers, such joining linkers independently selected from SEQ ID NOs: 4 to 50. In embodiments, the linker comprises two or more joining linkers each joining linker independently selected from SEQ ID NOs: 4 to 50; wherein one joining linker is N terminal to the hinge-C_{H2}-C_{H3}-Fc domain and another joining linker is C terminal to the hinge-C_{H2}-C_{H3}-Fc domain.

In one aspect, the present disclosure provides a chimeric protein having a general structure of: N terminus-(a)-(b)-(c)-C terminus, or a nucleic acid encoding the same, wherein: (a) is a first domain comprising a portion of the extracellular domain of TNF receptor (TNFR2) that is capable of binding TNFa and/or capable of oligomerizing with a cellular TNF receptor; (b) is a linker adjoining the first domain and a second domain comprising a hinge-C_{H2}-C_{H3} Fc domain, and (c) is the second domain comprising a portion of the extracellular domain of Dectin-2 capable of binding an alpha-mannan.

In one aspect, the present disclosure provides a chimeric protein having a general structure of: N terminus-(a)-(b)-(c)-C terminus, or a nucleic acid encoding the same, wherein: (a) is a first domain comprising a portion of the extracellular domain of TNF receptor (TNFR2) capable of binding TNFa and/or capable of oligomerizing with a cellular TNF receptor, and comprising an amino acid sequence that is at least about 95% identical to the amino acid sequence of SEQ ID NO: 57; (b) is a linker adjoining the first domain and a second domain comprising a hinge-C_{H2}-C_{H3} Fc domain comprising an amino acid sequence that is at least about 95% identical to the amino acid sequence selected from SEQ ID NO: 1, SEQ ID NO: 2, SEQ ID NO: 3, and SED ID NO: 73, and (c) is the second domain comprising a portion of the extracellular domain of Dectin-2 capable of binding a ligand comprising an alpha-mannan, and comprising an amino acid sequence that is at least about 95% identical to the amino acid sequence of SEQ ID NO: 64 or SEQ ID NO: 65.

An illustrative TNFR2-Fc-Dectin-2 chimeric protein has the following sequence (the extracellular domain of TNFR2 is shown by an underline, a linker comprising a mutant Fc domain of human IgG1 is shown in boldface font, with mutations shown by an underline, a joining linker is shown in an underlined-boldface-italic font, and the C-type lectin binding domain (CLD) of Dectin-2 is shown in an italics font):

(SEQ ID NO: 72)
LPAQVAFTPYAPEPGSTCRLREYYDQTAQMCCSKCSPGQHAKVFCTKTSDTVCDSCEDSTYTQL
WNWVPECLSCGSRCSSDQVETQACTREQNRICTCRPGWYCALSKQEGCRLCAPLRKCRPGFGV
ARPGTETSDVVCKPCAPGTFSNTTSSTDICRPHQICNVVAIPGNASMDAVCTSTSPTRSMAPGAV
HLPQPVSTRSQHTQPTPEPSTAPSTSFLLPMGPSPPAEGSTGDEPKSCDKTHTCPPCPAPEAAG
GPSVFLFPPKPKDTLMISRTPEVTCVVVDVSHEDPEVKFNWYVDGVEVHNAKTKPREEQYASTY
RVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSV
MHEALHNHYTQKSLSLSPGKIEGRMDTYHFTYGETGKRLSELHSYHSSLTCFSEGTKVPAWGCC
PASWKSFGSSCYFISSEEKVWSKSEQNCVEMGAHLVVENTEAEQNFIVQQLNESFSYFLGLSDPQ
GNNNWQWIDKTPYEKNVRFWHLGEPNHSAEQCASIVFWKPTGWGWNDVICETRRNSICEMNKIY
L.

In embodiments, the chimeric protein comprises a variant of the TNFR2-Fc-Dectin-2 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 72.

An illustrative mouse TNFR2-Fc-Dectin2 chimeric protein has the following sequence (the extracellular domain of mouse TNFR2 is shown by an underline, a linker comprising a mutant Fc domain of mouse IgG1 is shown in boldface font, a joining linker is shown in an underlined-boldface-italic font, and the extracellular of Dectin2 is shown in an italics font):

(SEQ ID NO: 83)
VPAQVVLTPYKPEPGYECQISQEYYDRKAQMCCAKCPPGQYVKHFCNKTSDTVCADCEASMYTQ
VWNQFRTCLSCSSSCTTDQVEIRACTKQQNRVCACEAGRYCALKTHSGSCRQCMRLSKCGPGF
GVASSRAPNGNVLCKACAPGTFSDTTSSTDVCRPHRICSILAIPGNASTDAVCAPESPTLSAIPRTL
YVSQPEPTRSQPLDQEPGPSQTPSILTSLGSTPIIEQSTKGGVPRDCGCKPCICTVPEVSSVFIFPP
KPKDVLTITLTPKVTCVVVDISKDDPEVQFSWFVDDVEVHTAQTQPREEQFNSTFRSVSELPIMH
QDWLNGKEFKCRVNSAAFPAPIEKTISKTKGRPKAPQVYTIPPPKEQMAKDKVSLTCMITDFFPE
DITVEWQWNGQPAENYKNTQPIMDTDGSYFVYSKLNVQKSNWEAGNTFTCSVLHEGLHNHHTE
KSLSHSPGKIEGRMDIMDQPSRRLYELHTYHSSLTCFSEGTMVSEKMWGCCPNHWKSFGSSCYL
ISTKENFWSTSEQNCVQMGAHLVVINTEAEQNFITQQLNESLSYFLGLSDPQGNGKWQWIDDTPF
SQNVRFWHPHEPNLPEERCVSIVYWNPSKWGWNDVFCDSKHNSICEMKKIYL.

In embodiments, the chimeric protein comprises a variant of the mouse TNFR2-Fc-Dectin2 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 96%, or at least about 95%, or at least about 96%, or at least about 97%, or at least about 98%, or at least about 99% sequence identity with SEQ ID NO: 83.

In embodiments, where the chimeric protein comprises a portion of langerin, a joining linker preceding an Fc domain, the Fc domain, a joining linker following the Fc domain, and a portion of Clec7a, the chimeric protein may comprise the following structure:

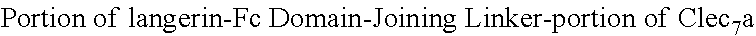

In embodiments, the chimeric protein comprises: a portion of langerin capable of binding a ligand comprising a sulfated glycan, a mannosylated glycan, a keratan sulfate (KS) and/or a beta-glucan, and comprising an amino acid sequence that is at least about 90%, or at least about 95%, or at least about 97%, or at least about 98%, or at least about 99% identical to the amino acid sequence of SEQ ID NO: 60 or SEQ ID NO: 61; a portion of Clec7a capable of binding a beta-1,3-linked and/or beta-1,6-linked glucan, and comprising an amino acid sequence that is at least about 90%, or at least about 95%, or at least about 97%, or at least about 98%, or at least about 99% identical to the amino acid sequence of SEQ ID NO: 58 or SEQ ID NO: 59; and a linker adjoining the extracellular domains. In embodiments, the hinge-C_{H2}-C_{H3} Fc domain is derived from IgG1 or IgG4, e.g., human IgG1 or IgG4. In embodiments, the hinge-C_{H2}-C_{H3} Fc domain comprises an amino acid sequence that is at least about 95% identical to the amino acid sequence of SEQ ID NO: 1, SEQ ID NO: 2, SEQ ID NO: 3, or SEQ ID NO: 73. In embodiments, the linker further comprises the linker comprises one or more joining linkers, such joining linkers independently selected from SEQ ID NOs: 4 to 50. In embodiments, the linker comprises two or more joining linkers each joining linker independently selected from SEQ ID NOs: 4 to 50; wherein one joining linker is N terminal to the hinge-C_{H2}-C_{H3}-Fc domain and another joining linker is C terminal to the hinge-C_{H2}-C_{H3}-Fc domain.

In one aspect, the present disclosure provides a chimeric protein having a general structure of: N terminus-(a)-(b)-(c)-C terminus, or a nucleic acid encoding the same, wherein: (a) is a first domain comprising a portion of the portion of langerin that is capable of binding a sulfated glycan, a mannosylated glycan, a keratan sulfate (KS) and/or a beta-glucan; (b) is a linker adjoining the first domain and a second domain comprising a hinge-C_{H2}-C_{H3} Fc domain, and (c) is the second domain comprising a portion of the extracellular domain of Clec7a capable of binding a beta-1,3-linked and/or beta-1,6-linked glucan.

In one aspect, the present disclosure provides a chimeric protein having a general structure of: N terminus-(a)-(b)-(c)-C terminus, or a nucleic acid encoding the same, wherein: (a) is a first domain comprising a portion of the portion of langerin capable of binding a sulfated glycan, a mannosylated glycan, a keratan sulfate (KS) and/or a beta-glucan, and comprising an amino acid sequence that is at least about 95% identical to the amino acid sequence of SEQ ID NO: 60 or SEQ ID NO: 61; (b) is a linker adjoining the first domain and a second domain comprising a hinge-C_{H2}-C_{H3} Fc domain comprising an amino acid sequence that is at least about 95% identical to the amino acid sequence selected from SEQ ID NO: 1, SEQ ID NO: 2, SEQ ID NO: 3, and SED ID NO: 73, and (c) is the second domain comprising a portion of the extracellular domain of Clec7a capable of binding a beta-1,3-linked and/or beta-1,6-linked glucan, and comprising an amino acid sequence that is at least about 95% identical to the amino acid sequence of SEQ ID NO: 58 or SEQ ID NO: 59.

An illustrative langerin-Fc-Clec7a chimeric protein has the following sequence (the extracellular domain of langerin is shown by an underline, a linker comprising a mutant Fc domain of human IgG1 is shown in boldface font, with mutations shown by an underline, a joining linker is shown in an underlined-boldface-italic font, and the extracellular of Clec7a is shown in an italics font):

(SEQ ID NO: 74)
PRFMGTISDVKTNVQLLKGRVDNISTLDSEIKKNSDGMEAAGVQIQMVNESLGYVRSQFLKLKTSVEKANA
QIQILTRSWEEVSTLNAQIPELKSDLEKASALNTKIRALQGSLENMSKLLKRQNDILQVVSQGWKYFKGNFY
YFSLIPKTWYSAEQFCVSRNSHLTSVTSESEQEFLYKTAGGLIYWIGLTKAGMEGDWSWVDDTPFNKVQS
VRFWIPGEPNNAGNNEHCGNIKAPSLQAWNDAPCDKTFLFICKRPYVPSEPEPKSCDKTHTCPPCPAPEA
AGGPSVFLFPPKPKDTLMISRTPEVTCVVVDVSHEDPEVKFNWYVDGVEVHNAKTKPREEQYASTYRVV
SVLTVLHQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHNHYTQKSLS
LSPGKIEGRMDTMAIWRSNSGSNTLENGYFLSRNKENHSQPTQSSLEDSVTPTKAVKTTGVLSSPCPPNW
IIYEKSCYLFSMSLNSWDGSKRQCWQLGSNLLKIDSSNELGFIVKQVSSQPDNSFWIGLSRPQTEVPWLWE
DGSTFSSNLFQIRTTATQENPSPNCVWIHVSVIYDQLCSVPSYSICEKKFSM.

In embodiments, the chimeric protein comprises a variant of the langerin-Fc-Clec7a chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 74.

An illustrative langerin (CLD)-Fc-Clec7a (CLD) chimeric protein has the following sequence (the C-type lectin binding domain (CLD) of langerin is shown by an underline, a linker comprising a mutant Fc domain of human IgG1 is shown in boldface font, with mutations shown by an underline, a joining linker is shown in an underlined-boldface-italic font, and the C-type lectin binding domain (CLD) of Clec7a is shown in an italics font):

(SEQ ID NO: 75)
QVVSQGWKYFKGNFYYFSLIPKTWYSAEQFCVSRNSHLTSVTSESEQEFLYKTAGGLIYWIGLTKAGMEG
DWSWVDDTPFNKVQSVRFWIPGEPNNAGNNEHCGNIKAPSLQAWNDAPCDKTFLFICKRPYVPSEPEPK
SCDKTHTCPPCPAPEAAGGPSVFLFPPKPKDTLMISRTPEVTCVVVDVSHEDPEVKFNWYVDGVEVHNA
KTKPREEQYASTYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRWQQGNVFSCS
VMHEALHNHYTQKSLSLSPGKIEGRMDSSPCPPNWIIYEKSCYLFSMSLNSWDGSKRQCWQLGSNLLKID
SSNELGFIVKQVSSQPDNSFWIGLSRPQTEVPWLWEDGSTFSSNLFQIRTTATQENPSPNCVWIHVSVIYD
QLCSVPSYSICEKKFSM.

In embodiments, the chimeric protein comprises a variant of the langerin-Fc-Clec7a chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 75.

In embodiments, where the chimeric protein comprises a portion of langerin, a joining linker preceding an Fc domain, the Fc domain, a joining linker following the Fc domain, and a portion of DC-SIGN, the chimeric protein may comprise the following structure:

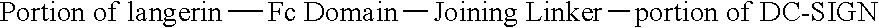

In embodiments, the chimeric protein comprises: a portion of langerin capable of binding a ligand comprising a sulfated glycan, a mannosylated glycan, a keratan sulfate (KS) and/or a beta-glucan, and comprising an amino acid sequence that is at least about 90%, or at least about 95%, or at least about 97%, or at least about 98%, or at least about 99% identical to the amino acid sequence of SEQ ID NO 60 or SEQ ID NO: 61; a portion of DC-SIGN capable of binding Intercellular Adhesion Molecule 2 (ICAM2) and/or Intercellular Adhesion Molecule 3 (ICAM3), and comprising an amino acid sequence that is at least about 90%, or at least about 95%, or at least about 97%, or at least about 98%, or at least about 99% identical to the amino acid sequence of SEQ ID NO: 62 or SEQ ID NO: 63; and a linker adjoining the extracellular domains. In embodiments, the hinge-C_{H2}-C_{H3} Fc domain is derived from IgG1 or IgG4, e.g., human IgG1 or IgG4. In embodiments, the hinge-C_{H2}-C_{H3} Fc domain comprises an amino acid sequence that is at least about 95% identical to the amino acid sequence of SEQ ID NO: 1, SEQ ID NO: 2, SEQ ID NO: 3, or SEQ ID NO: 73. In embodiments, the linker further comprises the linker comprises one or more joining linkers, such joining linkers independently selected from SEQ ID NOs: 4 to 50. In embodiments, the linker comprises two or more joining linkers each joining linker independently selected from SEQ ID NOs: 4 to 50; wherein one joining linker is N terminal to the hinge-C_{H2}-C_{H3}-Fc domain and another joining linker is C terminal to the hinge-C_{H2}-C_{H3}-Fc domain.

In one aspect, the present disclosure provides a chimeric protein having a general structure of: N terminus-(a)-(b)-(c)-C terminus, or a nucleic acid encoding the same, wherein: (a) is a first domain comprising a portion of the portion of langerin that is capable of binding a sulfated glycan, a mannosylated glycan, a keratan sulfate (KS) and/or a beta-glucan; (b) is a linker adjoining the first domain and a second domain comprising a hinge-C_{H2}-C_{H3} Fc domain, and (c) is the second domain comprising a portion of the extracellular domain of DC-SIGN capable of binding Intercellular Adhesion Molecule 2 (ICAM2) and/or Intercellular Adhesion Molecule 3 (ICAM3).

In one aspect, the present disclosure provides a chimeric protein having a general structure of: N terminus-(a)-(b)-(c)-C terminus, or a nucleic acid encoding the same, wherein: (a) is a first domain comprising a portion of the portion of langerin capable of binding a sulfated glycan, a mannosylated glycan, a keratan sulfate (KS) and/or a beta-glucan, and comprising an amino acid sequence that is at least about 95% identical to the amino acid sequence of SEQ ID NO: 62 or SEQ ID NO: 63; (b) is a linker adjoining the first domain and a second domain comprising a hinge-C_{H2}-C_{H3} Fc domain comprising an amino acid sequence that is at least about 95% identical to the amino acid sequence selected from SEQ ID NO: 1, SEQ ID NO: 2, SEQ ID NO: 3, and SED ID NO: 73, and (c) is the second domain comprising a portion of the extracellular domain of DC-SIGN capable of binding Intercellular Adhesion Molecule 2 (ICAM2) and/or Intercellular Adhesion Molecule 3 (ICAM3), and comprising an amino acid sequence that is at least about 95% identical to the amino acid sequence of SEQ ID NO: 62 or SEQ ID NO: 63.

An illustrative langerin-Fc-DC-SIGN chimeric protein has the following sequence (the extracellular domain of langerin is shown by an underline, a linker comprising a mutant Fc domain of human IgG1 is shown in boldface font, with mutations shown by an underline, a joining linker is shown in an underlined-boldface-italic font, and the extracellular of DC-SIGN is shown in an italics font):

(SEQ ID NO: 76)
PRFMGTISDVKTNVQLLKGRVDNISTLDSEIKKNSDGMEAAGVQIQMVNESLGYVRSQFLKLKTSV
EKANAQIQILTRSWEEVSTLNAQIPELKSDLEKASALNTKIRALQGSLENMSKLLKRQNDILQVVSQ
GWKYFKGNFYYFSLIPKTWYSAEQFCVSRNSHLTSVTSESEQEFLYKTAGGLIYWIGLTKAGMEG
DWSWVDDTPFNKVQSVRFWIPGEPNNAGNNEHCGNIKAPSLQAWNDAPCDKTFLFICKRPYVPS
EPEPKSCDKTHTCPPCPAPEAAGGPSVFLFPPKPKDTLMISRTPEVTCVVVDVSHEDPEVKFNW
YVDGVEVHNAKTKPREEQYASTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGKIEGRMDQVSKVPSSISQEQSR
QDAIYQNLTQLKAAVGELSEKSKLQEIYQELTQLKAAVGELPEKSKLQEIYQELTRLKAAVGELPEK
SKLQEIYQELTWLKAAVGELPEKSKMQEIYQELTRLKAAVGELPEKSKQQEIYQELTRLKAAVGEL
PEKSKQQEIYQELTRLKAAVGELPEKSKQQEIYQELTQLKAAVERLCHPCPWEWTFFQGNCYFMS
NSQRNWHDSITACKEVGAQLVVIKSAEEQNFLQLQSSRSNRFTWMGLSDLNQEGTWQWVDGSP
LLPSFKQYWNRGEPNNVGEEDCAEFSGNGWNDDKCNLAKFWICKKSAASCSRDEEQFLSPAPA
TPNPPPA.

In embodiments, the chimeric protein comprises a variant of the langerin-Fc-DC-SIGN chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 76.

An illustrative langerin (CLD)-Fc-DC-SIGN (CLD) chimeric protein has the following sequence (the C-type lectin binding domain (CLD) of langerin is shown by an underline, a linker comprising a mutant Fc domain of human IgG1 is shown in boldface font, with mutations shown by an underline, a joining linker is shown in an underlined-boldface-italic font, and the C-type lectin binding domain (CLD) of DC-SIGN is shown in an italics font):

(SEQ ID NO: 77)
QVVSQGWKYFKGNFYYFSLIPKTWYSAEQFCVSRNSHLTSVTSESEQEFLYKTAGGLIYWIGLTKA
GMEGDWSWVDDTPFNKVQSVRFWIPGEPNNAGNNEHCGNIKAPSLQAWNDAPCDKTFLFICKR
PYVPSEPEPKSCDKTHTCPPCPAPEAAGGPSVFLFPPKPKDTLMISRTPEVTCVVVDVSHEDPEV
KFNWYVDGVEVHNAKTKPREEQYASTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGKIEGRMDHPCPWEWTFFQ
GNCYFMSNSQRNWHDSITACKEVGAQLVVIKSAEEQNFLQLQSSRSNRFTWMGLSDLNQEGTW
QWVDGSPLLPSFKQYWNRGEPNNVGEEDCAEFSGNGWNDDKCNLAKFWICK.

In embodiments, the chimeric protein comprises a variant of the langerin-Fc-DC-SIGN chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 77.

In embodiments, where the chimeric protein comprises a portion of langerin, a joining linker preceding an Fc domain, the Fc domain, a joining linker following the Fc domain, and a portion of Dectin-2, the chimeric protein may comprise the following structure:

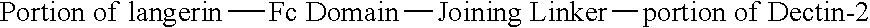

In embodiments, the chimeric protein comprises: a portion of langerin capable of binding a ligand comprising a sulfated glycan, a mannosylated glycan, a keratan sulfate (KS) and/or a beta-glucan, and comprising an amino acid sequence that is at least about 90%, or at least about 95%, or at least about 97%, or at least about 98%, or at least about 99% identical to the amino acid sequence of SEQ ID NO 60 or SEQ ID NO: 61; a portion of Dectin-2 capable of binding an alpha-mannan, and comprising an amino acid sequence that is at least about 90%, or at least about 95%, or at least about 97%, or at least about 98%, or at least about 99% identical to the amino acid sequence of SEQ ID NO: 64 or SEQ ID NO: 65; and a linker adjoining the extracellular domains. In embodiments, the hinge-C_{H2}-C_{H3} Fc domain is derived from IgG1 or IgG4, e.g., human IgG1 or IgG4. In embodiments, the hinge-C_{H2}-C_{H3} Fc domain comprises an amino acid sequence that is at least about 95% identical to the amino acid sequence of SEQ ID NO: 1, SEQ ID NO: 2, SEQ ID NO: 3, or SEQ ID NO: 73. In embodiments, the linker further comprises the linker comprises one or more joining linkers, such joining linkers independently selected from SEQ ID NOs: 4 to 50. In embodiments, the linker comprises two or more joining linkers each joining linker independently selected from SEQ ID NOs: 4 to 50; wherein one joining linker is N terminal to the hinge-C_{H2}-C_{H3}-Fc domain and another joining linker is C terminal to the hinge-C_{H2}-C_{H3}-Fc domain.

In one aspect, the present disclosure provides a chimeric protein having a general structure of: N terminus-(a)-(b)-(c)-C terminus, or a nucleic acid encoding the same, wherein: (a) is a first domain comprising a portion of the portion of langerin that is capable of binding a sulfated glycan, a mannosylated glycan, a keratan sulfate (KS) and/or a beta-glucan; (b) is a linker adjoining the first domain and a second domain comprising a hinge-C_{H2}-C_{H3} Fc domain, and (c) is the second domain comprising a portion of the extracellular domain of Dectin-2 capable of binding an alpha-mannan.

In one aspect, the present disclosure provides a chimeric protein having a general structure of: N terminus-(a)-(b)-(c)-C terminus, or a nucleic acid encoding the same, wherein: (a) is a first domain comprising a portion of the portion of langerin capable of binding a sulfated glycan, a mannosylated glycan, a keratan sulfate (KS) and/or a beta-glucan, and comprising an amino acid sequence that is at least about 95% identical to the amino acid sequence of SEQ ID NO: 64 or SEQ ID NO: 65; (b) is a linker adjoining the first domain and a second domain comprising a hinge-C_{H2}-C_{H3} Fc domain comprising an amino acid sequence that is at least about 95% identical to the amino acid sequence selected from SEQ ID NO: 1, SEQ ID NO: 2, SEQ ID NO: 3, and SED ID NO: 73, and (c) is the second domain comprising a portion of the extracellular domain of Dectin-2 capable of binding an alpha-mannan, and comprising an amino acid sequence that is at least about 95% identical to the amino acid sequence of SEQ ID NO: 64 or SEQ ID NO: 65.

An illustrative langerin-Fc-Dectin-2 chimeric protein has the following sequence (the extracellular domain of langerin is shown by an underline, a linker comprising a mutant Fc domain of human IgG1 is shown in boldface font, with mutations shown by an underline, a joining linker is shown in an underlined-boldface-italic font, and the extracellular of Dectin-2 is shown in an italics font):

(SEQ ID NO: 78)
PRFMGTISDVKTNVQLLKGRVDNISTLDSEIKKNSDGMEAAGVQIQMVNESLGYVRSQFLKLKTSV
EKANAQIQILTRSWEEVSTLNAQIPELKSDLEKASALNTKIRALQGSLENMSKLLKRQNDILQVVSQ
GWKYFKGNFYYFSLIPKTWYSAEQFCVSRNSHLTSVTSESEQEFLYKTAGGLIYWIGLTKAGMEG
DWSWVDDTPFNKVQSVRFWIPGEPNNAGNNEHCGNIKAPSLQAWNDAPCDKTFLFICKRPYVPS
EPEPKSCDKTHTCPPCPAPEAAGGPSVFLFPPKPKDTLMISRTPEVTCVVVDVSHEDPEVKFNW
YVDGVEVHNAKTKPREEQYASTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGKIEGRMDTYHFTYGETGKRLSEL
HSYHSSLTCFSEGTKVPAWGCCPASWKSFGSSCYFISSEEKVWSKSEQNCVEMGAHLVVENTEA
EQNFIVQQLNESFSYFLGLSDPQGNNNWQWIDKTPYEKNVRFWHLGEPNHSAEQCASIVFWKPT
GWGWNDVICETRRNSICEMNKIYL.

In embodiments, the chimeric protein comprises a variant of the langerin-Fc-Dectin-2 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 78.

An illustrative langerin (CLD)-Fc-Dectin-2 (CLD) chimeric protein has the following sequence (the C-type lectin binding domain (CLD) of langerin is shown by an underline, a linker comprising a mutant Fc domain of human IgG1 is shown in boldface font, with mutations shown by an underline, a joining linker is shown in an underlined-boldface-italic font, and the extracellular domain of Dectin-2 is shown in an italics font):

(SEQ ID NO: 79)
QVVSQGWKYFKGNFYYFSLIPKTWYSAEQFCVSRNSHLTSVTSESEQEFLYKTAGGLIYWIGLTKA
GMEGDWSWVDDTPFNKVQSVRFWIPGEPNNAGNNEHCGNIKAPSLQAWNDAPCDKTFLFICKR
PYVPSEPEPKSCDKTHTCPPCPAPEAAGGPSVFLFPPKPKDTLMISRTPEVTCVVVDVSHEDPEV
KFNWYVDGVEVHNAKTKPREEQYASTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGKIEGRMDTYHFTYGETGK
RLSELHSYHSSLTCFSEGTKVPAWGCCPASWKSFGSSCYFISSEEKVWSKSEQNCVEMGAHLVV
FNTEAEQNFIVQQLNESFSYFLGLSDPQGNNNWQWIDKTPYEKNVRFWHLGEPNHSAEQCASIVF
WKPTGWGWNDVICETRRNSICEMNKIYL.

In embodiments, the chimeric protein comprises a variant of the langerin-Fc-Dectin-2 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO:

79.

### The Nucleic Acids Encoding the Chimeric Proteins of the Present Disclosure

In various aspects, the present disclosure provides an isolated nucleic acid encoding any of the chimeric proteins disclosed herein or any component thereof.

In one aspect, the present disclosure relates to an isolated polynucleotide encoding a chimeric protein having a general structure of: N terminus-(a)-(b)-(c)-C terminus, wherein: (a) is a first domain comprising an extracellular domain of tumor necrosis factor (TNF) receptor 2 (TNFR2), or a variant or a fragment thereof that is capable of binding a TNFR2 ligand, (c) is a second domain comprising an extracellular domain selected from CLEC7a, or a variant or a fragment thereof that capable of binding a CLEC7a ligand, DC-SIGN (CD209), or a variant or a fragment thereof that capable of binding a DC-SIGN (CD209) ligand, DECTIN2 (CLEC6A), or a variant or a fragment thereof that capable of binding a DECTIN2 (CLEC6A) ligand, Langerin (CD207,CLC4K), or a variant or a fragment thereof that capable of binding a Langerin (CD207,CLC4K) ligand, CD69, (b) is a linker adjoining the first and second domains, wherein the linker comprises at least one cysteine residue capable of forming a disulfide bond and/or comprises a hinge-C_{H2}-C_{H3} Fc domain. In embodiments, the TNFR2 ligand is TNFα. In embodiments, the CLEC7a ligand is a beta-1,3-linked and/or beta-1,6-linked glucan. In embodiments, the DC-SIGN (CD209) ligand is a Intercellular Adhesion Molecule 2 (ICAM2) and/or Intercellular Adhesion Molecule 3 (ICAM3). In embodiments, the DECTIN2 (CLEC6A) ligand is an alpha-mannan. In embodiments, the Langerin (CD207,CLC4K) ligand is a sulfated glycan, a mannosylated glycan, a keratan sulfate (KS) and/or a beta-glucan. In embodiments, the CD69 ligand is Galectin-1 (Gal-1) or the S100A8/S100A9 complex. In embodiments, the isolated polynucleotide is or comprises an mRNA. In embodiments, the isolated polynucleotide is or comprises an mRNA that is modified according to any of the embodiments disclosed herein.

In one aspect, the present disclosure provides an isolated polynucleotide encoding a chimeric protein having a general structure of: N terminus-(a)-(b)-(c)-C terminus, or a nucleic acid encoding the same, wherein: (a) is a first domain comprising a portion of TNF receptor (TNFR2) that is capable of binding TNFa and/or capable of oligomerizing with a cellular TNF receptor; (b) is a linker adjoining the first domain and a second domain, optionally comprising a hinge-C_{H2}-C_{H3} Fc domain, and (c) is a second domain comprising a portion of a C-type lectin receptor (CLR) capable of binding a ligand. In embodiments, the CLR is selected from C-Type lectin domain containing 7A(Clec7A), langerin, dendritic cell-specific intercellular adhesion molecule-3-grabbing non-integrin (DC-SIGN), and dendritic cell-associated C-type lectin-2 (Dectin-2).

In one aspect, the present disclosure provides an isolated polynucleotide encoding a chimeric protein having a general structure of: N terminus-(a)-(b)-(c)-C terminus, or a nucleic acid encoding the same, wherein: (a) is a first domain comprising a portion of the extracellular domain of TNF receptor (TNFR2) that is capable of binding TNFa and/or capable of oligomerizing with a cellular TNF receptor; (b) is a linker adjoining the first domain and a second domain comprising a hinge-C_{H2}-C_{H3} Fc domain, and (c) is the second domain comprising a portion of the extracellular domain of C-Type Lectin Domain Containing 7A(Clec7A) capable of binding a beta-1,3-linked and/or beta-1,6-linked glucan.

In one aspect, the present disclosure provides an isolated polynucleotide encoding a chimeric protein having a general structure of: N terminus-(a)-(b)-(c)-C terminus, or a nucleic acid encoding the same, wherein: (a) is a first domain comprising a portion of the extracellular domain of TNF receptor (TNFR2) that is capable of binding TNFa and/or capable of oligomerizing with a cellular TNF receptor; (b) is a linker adjoining the first domain and a second domain comprising a hinge-C_{H2}-C_{H3} Fc domain, and (c) is the second domain comprising a portion of the extracellular domain of langerin capable of binding a sulfated glycan, a mannosylated glycan, a keratan sulfate (KS) and/or a beta-glucan.

In one aspect, the present disclosure provides an isolated polynucleotide encoding a chimeric protein having a general structure of: N terminus-(a)-(b)-(c)-C terminus, or a nucleic acid encoding the same, wherein: (a) is a first domain comprising a portion of the extracellular domain of TNF receptor (TNFR2) that is capable of binding TNFa and/or capable of oligomerizing with a cellular TNF receptor; (b) is a linker adjoining the first domain and a second domain comprising a hinge-C_{H2}-C_{H3} Fc domain, and (c) is the second domain comprising a portion of the extracellular domain of DC-SIGN capable of binding an Intercellular Adhesion Molecule 2 (ICAM2) and/or Intercellular Adhesion Molecule 3 (ICAM3).

In one aspect, the present disclosure provides an isolated polynucleotide encoding a chimeric protein having a general structure of: N terminus-(a)-(b)-(c)-C terminus, or a nucleic acid encoding the same, wherein: (a) is a first domain comprising a portion of the extracellular domain of TNF receptor (TNFR2) that is capable of binding TNFa and/or capable of oligomerizing with a cellular TNF receptor; (b) is a linker adjoining the first domain and a second domain comprising a hinge-C_{H2}-C_{H3} Fc domain, and (c) is the second domain comprising a portion of the extracellular domain of Dectin-2 capable of binding an alpha-mannan.

In one aspect, the present disclosure provides a chimeric protein having a general structure of: N terminus-(a)-(b)-(c)-C terminus, or a nucleic acid encoding the same, wherein: (a) is a first domain comprising a portion of a first C-type lectin receptor (CLR) capable of binding its ligand; (b) is a linker adjoining the first domain and a second domain comprising a hinge-C_{H2}-C_{H3} Fc domain, and (c) is the second domain comprising a portion of a second C-type lectin receptor (CLR) capable of binding its ligand.

In one aspect, the present disclosure provides a chimeric protein having a general structure of: N terminus-(a)-(b)-(c)-C terminus, or a nucleic acid encoding the same, wherein: (a) is a first domain comprising a portion of the portion of langerin that is capable of binding a sulfated glycan, a mannosylated glycan, a keratan sulfate (KS) and/or a beta-glucan; (b) is a linker adjoining the first domain and a second domain comprising a hinge-C_{H2}-C_{H3} Fc domain, and (c) is the second domain comprising a portion of the extracellular domain of Clec7a capable of binding a beta-1,3-linked and/or beta-1,6-linked glucan.

In one aspect, the present disclosure provides a chimeric protein having a general structure of: N terminus-(a)-(b)-(c)-C terminus, or a nucleic acid encoding the same, wherein: (a) is a first domain comprising a portion of the portion of langerin that is capable of binding a sulfated glycan, a mannosylated glycan, a keratan sulfate (KS) and/or a beta-glucan; (b) is a linker adjoining the first domain and a second domain comprising a hinge-C_{H2}-C_{H3} Fc domain, and (c) is the second domain comprising a portion of the extracellular domain of DC-SIGN capable of binding Intercellular Adhesion Molecule 2 (ICAM2) and/or Intercellular Adhesion Molecule 3 (ICAM3).

In one aspect, the present disclosure provides a chimeric protein having a general structure of: N terminus-(a)-(b)-(c)-C terminus, or a nucleic acid encoding the same, wherein: (a) is a first domain comprising a portion of the portion of langerin that is capable of binding a sulfated glycan, a mannosylated glycan, a keratan sulfate (KS) and/or a beta-glucan; (b) is a linker adjoining the first domain and a second domain comprising a hinge-C_{H2}-C_{H3} Fc domain, and (c) is the second domain comprising a portion of the extracellular domain of Dectin-2 capable of binding an alpha-mannan.

In one aspect, the present disclosure provides an isolated polynucleotide encoding the chimeric protein of any of the embodiments disclosed herein.

In one aspect, the present disclosure provides a host cell comprising the vector of any of the embodiments disclosed herein. In one aspect, the present disclosure provides a host cell comprising an RNA (without limitations, e.g., mmRNA) encoring the chimeric protein of any of the embodiments disclosed herein. A host cell comprising the nucleic acid, e.g., the mmRNA of any of the embodiments disclosed herein.

In embodiments, the polynucleotide is RNA, optionally, an mRNA. In embodiments, the polynucleotide is codon optimized.

In embodiments, the polynucleotide is or comprises an mRNA or a modified mRNA (mmRNA). In embodiments, the polynucleotide may include a polynucleotide modification including, but not limited to, a nucleoside modification. In embodiments, the polynucleotide is or comprises an mmRNA. In embodiments, the mmRNA comprises one or more nucleoside modifications. In embodiments, the nucleoside modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2, N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof.

In embodiments, the polypeptide the at least one chemically modified nucleoside is selected from pseudouridine (Y), N1-methylpseudouridine (m1Ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1Ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methylinosine (m11), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and two or more combinations thereof.

In embodiments, the mmRNA does not cause a substantial induction of the innate immune response of a cell into which the mmRNA is introduced. In embodiments, the modification in the mmRNA enhance one or more of the efficiency of production of the chimeric protein, intracellular retention of the mmRNA, and viability of contacted cells, and possess reduced immunogenicity.

In embodiments, the mmRNA has a length sufficient to include an open reading frame encoding the chimeric protein of the present disclosure.

Modified mRNA need not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of a nucleic acid such that the function of the nucleic acid is not substantially decreased. A modification may also be a 5′ or 3′ terminal modification. The nucleic acids may contain at a minimum one and at maximum 100% modified nucleotides, or any intervening percentage, such as at least about 50% modified nucleotides, at least about 80% modified nucleotides, or at least about 90% modified nucleotides. In embodiments, the mmRNA may contain a modified pyrimidine such as uracil or cytosine. In embodiments, at least about 5%, at least about 10%, at least about 25%, at least about 50%, In embodiments, the modified uracil may be replaced by a compound having a single unique structure, or can be replaced by a plurality of compounds having different structures disclosed above (e.g., same mmRNA may contain 2, 3, 4 or more types of uniquely modified uracil). In embodiments, at least about 5%, at least about 10%, at least about 25%, at least about 50%, at least about 80%, at least about 90% or 100% of the cytosine in the nucleic acid may be replaced with a modified cytosine. The modified cytosine can be replaced by a compound having a single unique structure, or can be replaced by a plurality of compounds having different structures disclosed above (e.g., same mmRNA may contain 2, 3, 4 or more types of uniquely modified cytosine).

In embodiments, the mmRNA comprises at least one chemically modified nucleoside. In embodiments, wherein the at least one chemically modified nucleoside is selected from pseudouridine (Y), N1-methylpseudouridine (m1\′), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1Ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methylinosine (m11), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and two or more combinations thereof. In embodiments, the mmRNA comprises at least one chemically modified nucleoside, wherein the at least one chemically modified nucleoside is selected from pseudouridine, N1-methylpseudouridine, 5-methylcytosine, 5-methoxyuridine, and a combination thereof. In embodiments, the mmRNA comprises at least one chemically modified nucleoside is N1-methylpseudouridine. In embodiments, the mmRNA is fully modified with chemically-modified uridines. In embodiments, the mmRNA is a fully modified N1-methylpseudouridine mRNA. Additional chemical modifications are disclosed in US Patent Application Publication No. 20190111003, the entire contents of which are hereby incorporated by reference.

In embodiments, modified nucleosides include pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4 thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In embodiments, modified nucleosides include 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2, N2-dimethyl-6-thio-guanosine. In embodiments, the nucleotide can be modified on the major groove face and can include replacing hydrogen on C-5 of uracil with a methyl group or a halo group.

In embodiments, a modified nucleoside is 5′-O-(1-Thiophosphate)-Adenosine, 5′-O-(1-Thiophosphate)-Cytidine, 5′-O-(1-Thiophosphate)-Guanosine, 5′-O-(1-Thiophosphate)-Uridine or 5′-O-(1-Thiophosphate)-Pseudouridine.

Further examples of modified nucleotides and modified nucleotide combinations are disclosed in U.S. Pat. Nos. 8,710,200; 8,822,663; 8,999,380; 9,181,319; 9,254,311; 9,334,328; 9,464,124; 9,950,068; 10,626,400; 10,808,242; 11,020,477, and US Patent Application Publication Nos. 20220001026, 20210318817, 20210283262, 20200360481, 20200113844, 20200085758, 20170204152, 20190114089, 20190114090, 20180369374, 20180318385, 20190111003, and PCT International Application Publication Nos. WO/2017112943, WO 2014/028429, WO 2017/201325 the entire contents of which are hereby incorporated by reference. The methods for synthesizing the modified mRNA are disclosed, e.g., in US Patent Application Publication Nos. 20170204152, the entire contents of which are hereby incorporated by reference.

In embodiments, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the cytosine residues of the mmRNA are replaced by a modified cytosine residues. In embodiments, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of the uracil residues of the mmRNA are replaced by a modified uracil residues.

In embodiments, the mmRNA further comprises a 5′ untranslated region (UTR) and/or a 3′-UTR, wherein either or both may independently contain one or more different nucleoside modifications. In such embodiments, nucleoside modifications may also be present in the translatable region. In embodiments, the mmRNA further comprises a Kozak sequence. In embodiments, the mmRNA further comprises a internal ribosome entry site (IRES).

In embodiments, the mmRNA further comprises a 5′-cap and/or a poly A tail.

In embodiments, the 5′-cap contains a 5′-5′-triphosphate linkage between the 5′-most nucleotide and guanine nucleotide. In embodiments, the 5′-cap comprises a methylation of the ultimate and penultimate most 5′-nucleotides on the 2′-hydroxyl group. In embodiments, the 5′-cap facilitates binding the mRNA Cap Binding Protein (CBP), confers mRNA stability in the cell and/or confers translation competency.

In embodiments, the poly-A tail is greater than about 30 nucleotides, or greater than about 40 nucleotides in length. In embodiments, the poly-A tail at least about 40 nucleotides, or at least about 45 nucleotides, or at least about 55 nucleotides, or at least about 60 nucleotides, or at least about 80 nucleotides, or at least about 90 nucleotides, or at least about 100 nucleotides, or at least about 120 nucleotides, or at least about 140 nucleotides, or at least about 160 nucleotides, or at least about 180 nucleotides, or at least about 200 nucleotides, or at least about 250 nucleotides, or at least about 300 nucleotides, or at least about 350 nucleotides, or at least about 400 nucleotides, or at least about 450 nucleotides, or at least about 500 nucleotides, or at least about 600 nucleotides, or at least about 700 nucleotides, or at least about 800 nucleotides, or at least about 900 nucleotides, or at least about 1000 nucleotides in length.

In embodiments, the mmRNA comprises a 3′ untranslated region (UTR). In embodiments, the 3′ UTR comprises a nucleic acid sequence at least 90%, 95%, 96%, 97%, 98%, 99%, or 100% identical to a sequence listed in Table 4 of US Patent Application Publication No. 20190114089, which is incorporated herein in its entirety. In embodiments, the 3′ UTR comprises at least one microRNA-122 (miR-122) binding site, wherein the miR-122 binding site is a miR-122-3p binding site or a miR-122-5-binding site. In embodiments, the mmRNA comprises a nucleic acid sequence comprising a miRNA binding site. In some embodiments, the miRNA binding site binds to miR-122. In a particular embodiment, the miRNA binding site binds to miR-122-3p or miR-122-5p. In embodiments, the mmRNA comprises at least two, at least three, at least four, at least five, at least six, at least seven, at least eight, at least nine, or at least ten miRNA binding sites.

In embodiments, the miRNA binding site is inserted within the 3′ UTR. In embodiments, the polynucleotide is DNA. In embodiments, the further comprises a spacer sequence between the open reading frame and the miRNA binding site. In one aspect, the spacer sequence comprises at least about 10 nucleotides, at least about 20 nucleotides, at least about 30 nucleotides, at least about 40 nucleotides, at least about 50 nucleotides, at least about 60 nucleotides, at least about 70 nucleotides, at least about 80 nucleotides, at least about 90 nucleotides, or at least about 100 nucleotides.

In embodiments, the mmRNA further comprises a 5′ UTR. In embodiments, the 5′ UTR comprises a nucleic acid sequence at least 90%, 95%, 96%, 97%, 98%, 99%, or 100% identical to a sequence listed in Table 3 of US Patent Application Publication No. 20190114089, or a sequence disclosed in PCT International Application Publication Nos. WO 2017/201325 and WO 2014/164253, each of which is incorporated herein in its entirety. In embodiments, the 5′ UTR bears features, which play roles in translation initiation. In embodiments, the 5′ UTR harbors signatures like Kozak sequences which are commonly known to be involved in the process by which the ribosome initiates translation of many genes. In embodiments, the 5′ UTR forms secondary structures which are involved in elongation factor binding. In embodiments, the 5′ UTR of mRNA known to be upregulated, such as c-myc, may be used to enhance expression of a nucleic acid molecule, such as a polynucleotides. In embodiments, the 5′ UTR of mRNA known to be upregulated in liver and/or spleen may be used to enhance expression of a nucleic acid molecule, such as a polynucleotides, in liver and/or spleen.

In embodiments, at least one of the regions of linked nucleosides of A comprises a sequence of linked nucleosides which functions as a 5′ UTR and at least one of the regions of linked nucleosides of C comprises a sequence of linked nucleosides which functions as a 3′ UTR. In embodiments, the 5′ UTR and the 3′ UTR are from the same or different species. In embodiments, the 5′ UTR and the 3′ UTR may be the native untranslated regions from different proteins from the same or different species. In embodiments, the 5′ UTR and the 3′ UTR may have synthetic sequences.

In embodiments, the mmRNA further comprises a 3′ polyadenylation (polyA tail).

In embodiments, the mmRNA further comprises a 5′ terminal cap. In embodiments, the 5′ terminal cap is a Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

In embodiments, the polynucleotide is in vitro transcribed (IVT). In embodiments, the polynucleotide is chimeric. In embodiments, the polynucleotide is circular.

In embodiments, the polynucleotide is or comprises DNA. In embodiments, the polynucleotide is or comprises a minicircle or a plasmid DNA. In embodiments, the plasmid DNA is devoid of any prokaryotic components.

In embodiments, the polynucleotide comprises a tissue-specific control element. In embodiments, the tissue-specific control element is a promoter or an enhancer. In embodiments, the plasmid DNA is an expression vector. In embodiments, the DNA is or comprises a minicircle. In embodiments, the minicircle is a circular molecule, which is optionally small. In embodiments, the minicircle utilizes a cellular transcription and translation machinery to produce an encoded gene product. In embodiments, the minicircle is devoid of any prokaryotic components. In embodiments, the minicircle only comprises substantially only sequences of mammalian origin (or those that have been optimized for mammalian cells). In embodiments, the minicircle lacks or has reduced amount of DNA sequence elements that are recognized by the innate immune system and/or toll-like receptors. In embodiments, the minicircle is produced by excising any bacterial components of from a parental plasmid, thereby making it smaller than a parental DNA sequence. In embodiments, the minicircle is of non-viral origin. In embodiments, the minicircle remains episomal. In embodiments, the minicircle does not replicate with a host cell. In embodiments, expression of the chimeric protein in non-dividing cells harboring a minicircle lasts for at least 2 days, or at least 4 days, or at least 6 days, or at least 8 days, or at least 10 days, or at least 12 days, or at least 14 days, or at least 16 days, or at least 18 days, or at least 20 days, or at least 22 days, or at least 24 days, or longer in dividing cells. In embodiments, expression of the chimeric protein in non-dividing cells harboring a minicircle lasts for at least 4 days, or at least 6 days, or at least 8 days, or at least 10 days, or at least 1 week, or at least 2 weeks, or at least 3 weeks, or at least 4 weeks, or at least 5 weeks, or at least 6 weeks, or at least 1 month, or at least 2 months, or at least 3 months, or at least 4 months, or at least 5 months, or at least 6 months, or at least 8 months, or longer in dividing cells.

In embodiments, the mmRNAs of the present disclosure are produced by means available in the art, including but not limited to in vitro transcription (IVT) and synthetic methods. Enzymatic IVT, solid-phase, liquid-phase, combined synthetic methods, small region synthesis, and ligation methods may be utilized. In embodiments, mmRNAs are made using IVT enzymatic synthesis methods. Methods of making polynucleotides by IVT are known in the art and are described in International Application PCT International Patent Publication No. WO2013151666, the contents of which are incorporated herein by reference in their entirety. Accordingly, the present disclosure also includes polynucleotides, e.g., DNA, constructs and vectors that may be used to in vitro transcribe an mRNA described herein.

In embodiments, the polynucleotide is DNA. In embodiments, the polynucleotide comprises a skin-specific control element. In embodiments, the skin-specific control element is a skin-specific promoter selected from a keratin 5 (K5) promoter, a keratin 6 (K6) promoter, a keratin 14 (K14) promoter, a keratin 16 (K16) promoter, an alpha-1 (I) collagen promoter, a filaggrin promoter, a loricrin promoter, an involucrin promoter, a tyrosinase promoter, and an αV integrin promoter, which can be constructed as described in, e.g., U.S. Patent Application Publication Nos. 2002/0100068, 2003/0158137, 2005/0043235, 2007/0142282, 2008/0147045, 2010/0154068, 2020/0085021, each of which is incorporated herein by reference in its entirety . . .

In one aspect, the present disclosure provides a vector comprising the polynucleotide of any one of the embodiments disclosed herein. In embodiments, the chimeric protein can be provided as an expression vector. In embodiments, the expression vector is a DNA expression vector or an RNA expression vector. In embodiments, the expression vector is a viral expression vector. In embodiments, the expression vector is a non-viral expression vector (without limitation, e.g. a plasmid).

In embodiments, the present non-viral vectors are linear or circular DNA molecules that comprise a polynucleotide encoding a polypeptide and is operably linked to control sequences, wherein the control sequences provide for expression of the polynucleotide encoding the polypeptide. In embodiments, the non-viral vector comprises a promoter sequence, and transcriptional and translational stop signal sequences. In embodiments, the expression vector may include, among others, chromosomal and episomal vectors, e.g., vectors derived from bacterial plasmids, from transposons, from yeast episomes, from insertion elements, from yeast chromosomal elements, and vectors derived from combinations thereof. The present constructs may contain control regions that regulate as well as engender expression.

A vector generally comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In embodiments, the expression vector is an autonomously replicating plasmid or a virus (e.g. AAV vectors). In embodiments, the expression vector is non-plasmid and non-viral compounds that facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

In embodiments, the polynucleotide or cell therapy may employ expression vectors, which comprise the isolated polynucleotide encoding the chimeric protein operably linked to an expression control region that is functional in the host cell. The expression control region is capable of driving expression of the operably linked encoding nucleic acid such that the chimeric protein is produced in a human cell transformed with the expression vector. Expression control regions are regulatory polynucleotides (sometimes referred to herein as elements), such as promoters and enhancers, that influence expression of an operably linked nucleic acid. An expression control region of an expression vector is capable of expressing operably linked encoding nucleic acid in a human cell. In an embodiment, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. In various embodiments, the chimeric protein expression is inducible or repressible. Typically, the amount of increase or decrease conferred by such elements is proportional to the amount of signal present; the greater the amount of signal, the greater the increase or decrease in expression.

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription-initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122 (3): 322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), Flp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family, and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences including CRISPR/CAS9, zinc finger, TALEN, and meganuclease gene-editing technologies.

### Pharmaceutical Compositions

In one aspect, the present disclosure provides a pharmaceutical composition comprising a pharmaceutically acceptable excipient or carrier, and the chimeric protein of any of the embodiments disclosed herein, the isolated polynucleotide of any of the embodiments disclosed herein, the mmRNA of any of the embodiments disclosed herein, or the vector of any of the embodiments disclosed herein, or the host cell of any of the embodiments disclosed herein. In embodiments, the pharmaceutical composition comprises the mmRNA of any of the embodiments disclosed herein.

Suitable pharmaceutical compositions are disclosed in U.S. Pat. Nos. 8,710,200; 8,822,663; 8,999,380; 9,181,319; 9,254,311; 9,334,328; 9,464,124; 9,950,068; 10,626,400; 10,808,242; 11,020,477, US Patent Application Publication Nos. 20220001026, 20210318817, 20210283262, 20200360481, 20200113844, 20200085758, 20170204152, 20190114089, 20190114090, 20180369374, 20180318385, 20190111003, and PCT International Application Publication Nos. WO/2017112943, WO 2014/028429, WO 2017/201325 the entire contents of which are hereby incorporated by reference.

In one aspect, the present disclosure relates to a pharmaceutical composition comprising an isolated modified mRNA (mmRNA) encoding a heterologous chimeric protein having an amino acid sequence that has at least about 95% sequence identity with an amino acid sequence selected from SEQ ID NOs: 80-93. In embodiments, the carrier is mmRNA comprises a modification (e.g., an RNA element), wherein the modification provides a desired translational regulatory activity. Such modifications are described in PCT Application No. PCT International Application Publication No. WO2018213789, the entire contents of which are herein incorporated by reference.

In embodiments, the mmRNA further comprises a 3′ untranslated region (UTR). In embodiments, the 3′ UTR comprises at least one microRNA-122 (miR-122) binding site. In embodiments, the miR-122 binding site is a miR-122-3p binding site or a miR-122-5-binding site. In embodiments, the mmRNA further comprises a spacer sequence between the open reading frame and the miRNA binding site. In embodiments, the spacer sequence comprises at least about 10 nucleotides, at least about 20 nucleotides, at least about 30 nucleotides, at least about 40 nucleotides, at least about 50 nucleotides, at least about 60 nucleotides, at least about 70 nucleotides, at least about 80 nucleotides, at least about 90 nucleotides, or at least about 100 nucleotides.

In embodiments, the mmRNA further comprises a 5′ UTR. In embodiments, the 5′ UTR harbors a Kozak sequence and/or forms a secondary structure that stimulate elongation factor binding.

In embodiments, the mmRNA further comprises a 5′ terminal cap. In embodiments, the 5′ terminal cap is a Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

In any of the embodiments disclosed herein, the mmRNA may comprise one or more modifications. In any of the embodiments disclosed herein, the mmRNA may comprise at least one modification. In embodiments, the modification is nucleoside modification. In embodiments, the modification is a base modification. In embodiments, the modification is a sugar-phosphate backbone modification.

In embodiments, the modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2, N2-dimethyl-6-thio-guanosine, and a combination of any two or more thereof. In embodiments, the modifications are selected from pseudouridine (Y), N1-methylpseudouridine (m1Ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1Ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methylinosine (m1l), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine, and a combination of any two or more thereof. In embodiments, modification is selected from pseudouridine, N1-methylpseudouridine, 5-methylcytosine, 5-methoxyuridine, and a combination thereof.

In embodiments, the mmRNA comprises at least one N1-methylpseudouridine. In embodiments, the mmRNA is fully modified with chemically-modified uridines. In embodiments, the mmRNA is a fully modified with N1-methylpseudouridine.

In embodiments, the modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine or a combination of any two or more thereof.

In embodiments, the modifications are selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In embodiments, the modifications are selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In embodiments, the modifications are selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2, N2-dimethyl-6-thio-guanosine. In embodiments, the modifications are present on the major groove face. In embodiments, a hydrogen on C-5 of uracil is replaced with a methyl group or a halo group.

In embodiments, the mmRNA further comprises one or more modifications selected from 5′-O-(1-Thiophosphate)-Adenosine, 5′-O-(1-Thiophosphate)-Cytidine, 5′-O-(1-Thiophosphate)-Guanosine, 5′-O-(1-Thiophosphate)-Uridine and 5′-O-(1-Thiophosphate)-Pseudouridine.

In embodiments, the pharmaceutical composition is formulated as a lipid nanoparticle (LNP), a lipoplex, or a liposome. In embodiments, the pharmaceutical composition is formulated as a lipid nanoparticle (LNP). In embodiments, the mmRNAs described herein may be formulated in a cationic oil-in-water emulsion where the emulsion particle comprises an oil core and a cationic lipid that can interact with the mRNA anchoring the molecule to the emulsion particle. In embodiments, the mRNAs described herein may be formulated in a water-in-oil emulsion comprising a continuous hydrophobic phase in which the hydrophilic phase is dispersed. Exemplary emulsions can be made by the methods described in PCT International Application Publication Nos. WO2012006380 and WO201087791, each of which is herein incorporated by reference in its entirety.

In some embodiments, nucleic acids of the invention (e.g., mRNA) are formulated in a lipid nanoparticle (LNP). Lipid nanoparticles comprise typically comprise ionizable cationic lipid, non-cationic lipid, sterol and PEG lipid components along with the nucleic acid cargo of interest. The lipid nanoparticles of the invention can be generated using components, compositions, and methods as are disclosed, e.g., in PCT International Application Publication Nos. WO2021231854, WO2021050986, WO2021055833, WO2021213924, WO2021055849, WO2021214204, WO2021188969, WO2021055835, WO2020061284, WO2020061295, WO2017049245, WO2017031232, WO2017112865, WO2017218704, WO2017218704, WO2017099823, WO2017049074, WO2017117528, WO2017180917, WO2017075531, WO2017223135, WO2016118724, WO2015164674, WO2015038892, WO2014152211, and WO2013090648, the entire contents of each which are herein incorporated by reference. PEG-lipids selected from an ionizable lipid (e.g. as known in the art, such as those described in U.S. Pat. No. 8,158,601 and PCT International Application Publication Nos. WO2012099755 and WO 2015/130584, which are incorporated herein by reference in their entirety. The ionizable lipid may be selected from, but not limited to, a ionizable lipid described in International Publication Nos. WO2013086354 and WO2013116126; the contents of each of which are herein incorporated by reference in their entirety. In embodiments, the lipid may be a cleavable lipid such as those described in PCT International Publication No. WO2012170889, herein incorporated by reference in its entirety. In embodiments, the lipid may be synthesized by methods known in the art and/or as described in International Publication Nos. WO2013086354; the contents of each of which are herein incorporated by reference in their entirety. In embodiments, the LNP formulations described herein can additionally comprise a permeability enhancer molecule. Non-limiting permeability enhancer molecules are described in U.S. Publication No. US20050222064, herein incorporated by reference in its entirety.

In embodiments, the carrier is a lipidoid, a liposome, a lipoplex, a lipid nanoparticle, a polymeric nanoparticle, a peptide, a protein, a cell, a nanoparticle mimic, a nanotube, or a conjugate. In embodiments, the pharmaceutical composition is formulated as a lipid nanoparticle (LNP), a lipoplex, or a liposome. In embodiments, the pharmaceutical composition is formulated as a lipid nanoparticle (LNP). In embodiments, the lipid nanoparticles comprise lipids selected from an ionizable lipid (e.g. an ionizable cationic lipid selected from DLin-DMA, DLin-K-DMA, DLin-KC2-DMA, DLin-MC3-DMA, 98N12-5, and C12-200); a structural lipid (e.g. distearoylphosphatidylcholine (DSPC)); cholesterol, and a polyethyleneglycol (PEG)-lipid (e.g. a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18); 1,2-dioleoyl-3-trimethylammoniumpropane (DOTAP); dioleoylphosphatidylethanolamine (DOPE).

In embodiments, the pharmaceutical composition is formulated as a lipid nanoparticle (LNP). In embodiments, the LNP comprises a molar ratio of about 20-60% ionizable amino lipid, about 5-25% phospholipid, about 25-55% structural lipid, and about 0.5-1.5% PEG lipid. In embodiments, the LNP comprises a molar ratio of about 50% ionizable amino lipid, about 8-12% phospholipid, about 37-40% structural lipid, and about 1-2% PEG lipid. In embodiments, the lipid nanoparticles comprise lipids selected from an ionizable lipid (e.g., an ionizable cationic lipid selected from DLin-DMA, DLin-K-DMA, DLin-KC2-DMA, DLin-MC3-DMA, 98N12-5, and C12-200); a structural lipid (e.g., distearoylphosphatidylcholine (DSPC)); cholesterol, and a polyethyleneglycol (PEG)-lipid (e.g., , a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18)); 1,2-dioleoyl-3-trimethylammoniumpropane (DOTAP); dioleoylphosphatidylethanolamine (DOPE).

In embodiments, the lipid nanoparticles comprise (a) a cationic lipid comprising from 50 mol % to 85 mol % of the total lipid present in the particle; (b) a non-cationic lipid comprising from 13 mol % to 49.5 mol % of the total lipid present in the particle; and (c) a conjugated lipid that inhibits aggregation of particles comprising from 0.5 mol % to 2 mol % of the total lipid present in the particle. In embodiments, the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

In any of the embodiments disclosed herein, the pharmaceutical composition is formulated for parenteral administration. In any of the embodiments disclosed herein, the pharmaceutical composition is formulated for topical administration

In one aspect, the present disclosure provides a pharmaceutical composition comprising the mmRNA of any embodiment disclosed herein, or an LNP comprising an mmRNA of any embodiment disclosed herein. In embodiments, the pharmaceutical composition is formulated for parenteral administration.

In embodiments, the pharmaceutical composition comprises a modified mRNA (mmRNA) encoding a heterologous chimeric protein having an amino acid sequence that has at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence selected from SEQ ID NOs: 80-93. In embodiments, the pharmaceutical composition is formulated as an LNP comprising an ionizable amino lipid, a phospholipid, a structural lipid and a PEG lipid.

In embodiments, the pharmaceutical composition is formulated for parenteral administration. In embodiments, the pharmaceutical composition is formulated for topical, dermal, intradermal, intramuscular, intraperitoneal, intraarticular, intravenous, subcutaneous, intraarterial or transdermal administration. In embodiments, the pharmaceutical composition is formulated for topical administration.

In one aspect, the present disclosure provides a pharmaceutical composition comprising a pharmaceutically acceptable excipient or carrier, and the chimeric protein of any one of the embodiments disclosed herein, the isolated polynucleotide of any one of the embodiments disclosed herein, the vector of the embodiments disclosed herein, or the host cell of any of the embodiments disclosed herein. In embodiments, the pharmaceutical composition comprises the nucleic acid, e.g., the mmRNA of any one of the embodiments disclosed herein.

In embodiments, the lipid nanoparticles comprise lipids selected from an ionizable lipid (e.g. an ionizable cationic lipid selected from DLin-DMA, DLin-K-DMA, DLin-KC2-DMA, DLin-MC3-DMA, 98N12-5, and C12-200); a structural lipid (e.g. distearoylphosphatidylcholine (DSPC)); cholesterol, and a polyethyleneglycol (PEG)-lipid (e.g. a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18)); 1,2-dioleoyl-3-trimethylammoniumpropane (DOTAP); dioleoylphosphatidylethanolamine (DOPE); and the nucleic acid, e.g., the mmRNA.

In embodiments, the LNP comprises a molar ratio of about 20-60% ionizable amino lipid, about 5-25% phospholipid, about 25-55% structural lipid, and about 0.5-1.5% PEG lipid. In embodiments, the ionizable amino lipid comprises the following formula:

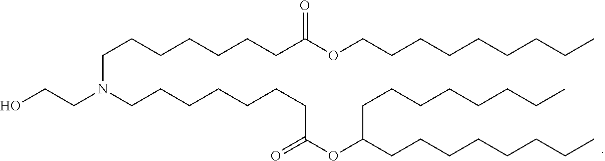 [SMILES: CCCCCCCCCOC(=O)CCCCCCCN(CCO)CCCCCCCC(=O)OC(CCCCCCCC)CCCCCCCC]

In embodiments, the lipid nanoparticles comprise lipids selected from an ionizable lipid; a structural lipid; cholesterol, and a polyethyleneglycol (PEG)-lipid; 1,2-dioleoyl-3-trimethylammoniumpropane (DOTAP); dioleoylphosphatidylethanolamine (DOPE); and the nucleic acid, e.g., the mmRNA. In embodiments, the ionizable lipid is an ionizable cationic lipid selected from DLin-DMA, DLin-K-DMA, DLin-KC2-DMA, DLin-MC3-DMA, 98N12-5, and C12-200. In embodiments, the polyethyleneglycol (PEG)-lipid is selected from a PEG-diacylglycerol (DAG), a PEG-dialkyloxypropyl (DAA), a PEG-phospholipid, a PEG-ceramide (Cer), or a mixture thereof, or a PEG-dilauryloxypropyl (e.g. C12, a PEG-dimyristyloxypropyl (C14), a PEG-dipalmityloxypropyl (C16), or a PEG-distearyloxypropyl (C18)).

In embodiments, the isolated polynucleotide is or comprises a conjugated polynucleotide sequence that is introduced into cells by various transfection methods such as, e.g., methods that employ lipid particles. In embodiments, a composition, including a gene transfer construct, comprises a delivery particle. In embodiments, the delivery particle comprises a lipid-based particle (e.g., a lipid nanoparticle (LNP)), cationic lipid, or a biodegradable polymer). Lipid nanoparticle (LNP) delivery of gene transfer construct provides certain advantages, including transient, non-integrating expression to limit potential off-target events and immune responses, and efficient delivery with the capacity to transport large cargos. LNPs have been used for delivery of small interfering RNA (siRNA) and mRNA, and for in vitro and in vivo delivering CRISPR/Cas9 components to hepatocytes and the liver. For example, U.S. Pat. No. 10,195,291 describes the use of LNPs for delivery of RNA interference (RNAi) therapeutic agents.

In embodiments, the composition in accordance with embodiments of the present disclosure is in the form of a LNP. In embodiments, the LNP comprises one or more lipids selected from 1,2-dioleoyl-3-trimethylammonium propane (DOTAP); N,N-dioleyl-N,N-dimethylammonium chloride (DODAC); N-(2,3-dioleyloxy) propyl)-N,N,N-trimethylammonium chloride (DOTMA); N,N-distearyl-N,N-dimethylammonium bromide (DDAB), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy (polyethylene glycol)-2000] (DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC). In embodiments, the composition can have a lipid and a polymer in various ratios, wherein the lipid can be selected from, e.g., DOTAP, DC-Chol, PC, Triolein, DSPE-PEG, and wherein the polymer can be, e.g., PEI or Poly Lactic-co-Glycolic Acid (PLGA). Any other lipid and polymer can be used additionally or alternatively. In embodiments, the ratio of the lipid and the polymer is about 0.5:1, or about 1:1, or about 1:1.5, or about 1:2, or about 1:2.5, or about 1:3, or about 3:1, or about 2.5:1, or about 2:1, or about 1.5:1, or about 1:1, or about 1:0.5.

In embodiments, the LNP comprises a cationic lipid, non-limiting examples of which include N,N-dioleyl-N,N-dimethylammonium chloride (DODAC), N,N-distearyl-N,N-dimethylammonium bromide (DDAB), N-(I-(2,3-dioleoyloxy) propyl)-N,N,N-trimethylammonium chloride (DOTAP), N-(I-(2,3-dioleyloxy) propyl)-N,N,N-trimethylammonium chloride (DOTMA), N,N-dimethyl-2,3-dioleyloxy) propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino) acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino) propane (DLin-MPZ), or 3-(N,N-Dilinoleylamino)-1,2-propanediol (DLinAP), 3-(N,N-Dioleylamino)-1,2-propanedio (DOAP), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino) butanoate (MC3), 1,1′-(2-(4-(2-((2-(bis(2-′) amino) ethyl) (2 hydroxydodecyl) amino) ethyl) piperazin-1-yl)ethylazanediyl)didodecan-2-ol (Tech G1), 1,2-Dilinoleyloxo-3-(2-N,N-dimethylamino) ethoxypropane (DLin-EG-DMA), or a mixture thereof.

In embodiments, the LNP comprises one or more molecules selected from polyethylenimine (PEI) and poly (lactic-co-glycolic acid) (PLGA), and N-Acetylgalactosamine (GalNAc), which are suitable for hepatic delivery. In embodiments, the LNP comprises a hepatic-directed compound as described, e.g., in U.S. Pat. No. 5,985,826, which is incorporated by reference herein in its entirety. GalNAc is known to target Asialoglycoprotein Receptor (ASGPR) expressed on mammalian hepatic cells. See Hu et al. Protein Pept Lett. 2014; 21 (10): 1025-30.

In some examples, the isolated polynucleotide can be formulated or complexed with PEI or a derivative thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives.

In embodiments, the LNP is a conjugated lipid, non-limiting examples of which include a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (C12, a PEG-dimyristyloxypropyl (C14), a PEG-dipalmityloxypropyl (C16), or a PEG-distearyloxypropyl (C18).

In embodiments, the LNP formulations may further contain a phosphate conjugate, which can increase in vivo circulation times and/or increase the targeted delivery of the nanoparticle. Phosphate conjugates can be made by the methods described in, e.g., PCT International Publication No. WO2013033438 or U.S. Pub. No. US20130196948. The LNP formulation can also contain a polymer conjugate (e.g., a water soluble conjugate) as described in, e.g., U.S. Publication Nos. US20130059360, US20130196948, and US20130072709, each of the references is herein incorporated by reference in its entirety.

In embodiments, the LNP formulations may comprise a carbohydrate carrier. As a non-limiting example, the carbohydrate carrier can include, but is not limited to, an anhydride-modified phytoglycogen or glycogen-type material, phytoglycogen octenyl succinate, phytoglycogen beta-dextrin, anhydride-modified phytoglycogen beta-dextrin (e.g., PCT International Publication No. WO2012109121, herein incorporated by reference in its entirety). In embodiments, the LNP formulations can be coated with a surfactant or polymer to improve the delivery of the particle. In some embodiments, the LNP can be coated with a hydrophilic coating such as, but not limited to, PEG coatings and/or coatings that have a neutral surface charge as described in U.S. Publication No. US20130183244, herein incorporated by reference in its entirety. In embodiments, the LNP formulations can be engineered to alter the surface properties of particles so that the lipid nanoparticles can penetrate the mucosal barrier as described in U.S. Pat. No. 8,241,670 or PCT International Publication No. WO2013110028, each of which is herein incorporated by reference in its entirety. In embodiments, the mucus penetrating LNP can be a hypotonic formulation comprising a mucosal penetration enhancing coating. The formulation can be hypotonic for the epithelium to which it is being delivered. Non-limiting examples of hypotonic formulations can be found in, e.g., PCT International Publication No. WO2013110028, herein incorporated by reference in its entirety.

In embodiments, an mmRNA described herein is formulated as a solid lipid nanoparticle (SLN), which can be spherical with an average diameter between 10 to 1000 nm. SLN possess a solid lipid core matrix that can solubilize lipophilic molecules and can be stabilized with surfactants and/or emulsifiers. Exemplary SLN can be those as described in PCT International Publication No. WO2013105101, herein incorporated by reference in its entirety.

In embodiments, a nanoparticle is a particle having a diameter of less than about 1000 nm. In embodiments, nanoparticles of the present disclosure have a greatest dimension (e.g., diameter) of about 500 nm or less, or about 400 nm or less, or about 300 nm or less, or about 200 nm or less, or about 100 nm or less. In embodiments, nanoparticles of the present disclosure have a greatest dimension ranging between about 50 nm and about 150 nm, or between about 70 nm and about 130 nm, or between about 80 nm and about 120 nm, or between about 90 nm and about 110 nm. In embodiments, the nanoparticles of the present disclosure have a greatest dimension (e.g., a diameter) of about 100 nm.

In embodiments, the chimeric protein or the therapeutic nanoparticle comprising mRNA can be formulated for sustained release, which, as used herein, refers to a pharmaceutical composition or compound that conforms to a release rate over a specific period of time. In embodiments, the period of time may include, but is not limited to, hours, days, weeks, months and years. As a non-limiting example, the sustained release nanoparticle of the mRNAs described herein can be formulated as disclosed in PCT International Publication No. WO2010075072 and U.S. Publication Nos. US20100216804, US20110217377, US20120201859 and US20130150295, each of which is herein incorporated by reference in their entirety.

In embodiments, the chimeric protein or the isolated polynucleotide or mmRNA (and/or additional agents) are included various formulations. Any chimeric protein, or the isolated polynucleotide or mmRNA (and/or additional agents) described herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In embodiments, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

In embodiments, the present disclosure provides an expression vector, comprising a nucleic acid encoding the chimeric protein described herein. In embodiments, the expression vector comprises DNA or RNA. In embodiments, the expression vector is a mammalian expression vector.

Both prokaryotic and eukaryotic vectors can be used for expression of the chimeric protein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and APL. Non-limiting examples of prokaryotic expression vectors may include the Agt vector series such as Agt11 (Huynh et al., in “DNA Cloning Techniques, Vol. I: A Practical Approach,” 1984, (D. Glover, ed.), pp. 49-78, IRL Press, Oxford), and the pET vector series (Studier et al., Methods Enzymol 1990, 185:60-89). Prokaryotic host-vector systems cannot perform much of the post-translational processing of mammalian cells, however. Thus, eukaryotic host-vector systems may be particularly useful. A variety of regulatory regions can be used for expression of the chimeric proteins in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the β-interferon gene, and the hsp70 gene (see, Williams et al., Cancer Res 1989, 49:2735-42; and Taylor et al., Mol Cell Biol 1990, 10:165-75). Heat shock promoters or stress promoters also may be advantageous for driving expression of the chimeric proteins in recombinant host cells.

In embodiments, expression vectors of the disclosure comprise a nucleic acid encoding the chimeric proteins (and/or additional agents), or a complement thereof, operably linked to an expression control region, or complement thereof, that is functional in a mammalian cell. The expression control region is capable of driving expression of the operably linked blocking and/or stimulating agent encoding nucleic acid such that the blocking and/or stimulating agent is produced in a human cell transformed with the expression vector.

Expression control regions are regulatory polynucleotides (sometimes referred to herein as elements), such as promoters and enhancers, that influence expression of an operably linked nucleic acid. An expression control region of an expression vector of the disclosure is capable of expressing operably linked encoding nucleic acid in a human cell. In embodiments, the cell is an epithelial cell. In embodiments, the cell is located in or near a lesion disorder caused by or associated with inflammation of the integumentary system. In embodiments, the cell is a non-tumor cell. In embodiments, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

In embodiments, the present disclosure contemplates the use of inducible promoters capable of effecting high level of expression transiently in response to a cue. For example, when in the proximity of a lesion disorder caused by or associated with inflammation of the integumentary system, a cell transformed with an expression vector for the chimeric protein (and/or additional agents) comprising such an expression control sequence is induced to transiently produce a high level of the agent by exposing the transformed cell to an appropriate cue. Illustrative inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941, each of which is incorporated herein by reference in its entirety.

Expression control regions and locus control regions include full-length promoter sequences, such as native promoter and enhancer elements, as well as subsequences or polynucleotide variants which retain all or part of full-length or non-variant function. As used herein, the term “functional” and grammatical variants thereof, when used in reference to a nucleic acid sequence, subsequence or fragment, means that the sequence has one or more functions of native nucleic acid sequence (e.g., non-variant or unmodified sequence).

As used herein, “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter. Examples of promoters that are expressed in the integumentary system include a keratin 5 (K5) promoter, a keratin 6 (K6) promoter, a keratin 14 (K14) promoter, a keratin 16 (K16) promoter, an alpha-1 (I) collagen promoter, a filaggrin promoter, a loricrin promoter, an involucrin promoter, a tyrosinase promoter, and an αV integrin promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Introns may also be included in expression constructs.

There are a variety of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a cell surface membrane protein from cells located in or near a lesion disorder caused by or associated with inflammation of the integumentary system. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990).

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122 (3): 322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), Flp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et al., supra), and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences encoding the chimeric proteins including CRISPR/CAS9, zinc finger, TALEN, and meganuclease gene-editing technologies.

In one aspect, the disclosure provides expression vectors for the expression of the chimeric proteins (and/or additional agents) that are viral vectors. Many viral vectors useful for gene therapy are known (see, e.g., Lundstrom, Trends Biotechnol., 21:1 17, 122, 2003. Illustrative viral vectors include those selected from Antiviruses (LV), retroviruses (RV), adenoviruses (AV), adeno-associated viruses (AAV), and a viruses, though other viral vectors may also be used. For in vivo uses, viral vectors that do not integrate into the host genome are suitable for use, such as a viruses and adenoviruses. Illustrative types of a viruses include Sindbis virus, Venezuelan equine encephalitis (VEE) virus, and Semliki Forest virus (SFV). For in vitro uses, viral vectors that integrate into the host genome are suitable, such as retroviruses, AAV, and Antiviruses. In embodiments, the disclosure provides methods of transducing a human cell in vivo, comprising contacting a cell that is located in or near a lesion disorder caused by or associated with inflammation of the integumentary system in vivo with a viral vector of the disclosure.

In embodiments, the present disclosure provides a host cell, comprising the expression vector comprising the chimeric protein described herein. In embodiments, the present disclosure provides a host cell comprising an RNA (without limitations, e.g., , mmRNA) encoring the chimeric protein of any of the embodiments disclosed herein.

Expression vectors can be introduced into host cells for producing the present chimeric proteins. Cells may be cultured in vitro or genetically engineered, for example. Useful mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et al., J Gen Virol 1977, 36:59); baby hamster kidney cells (e.g., BHK, ATCC CCL 10); Chinese hamster ovary-cells-DHFR (e.g., CHO, Urlaub and Chasin, Proc Natl Acad Sci USA 1980, 77:4216); DG44 CHO cells, CHO-K1 cells, mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cell types for expressing the chimeric proteins described herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, and human small cell lung carcinoma cell lines, SCLC #2 and SCLC #7.

Host cells can be obtained from normal or affected subjects, including healthy humans, patients suffering from inflammation of the integumentary system, and patients with an infectious disease, private laboratory deposits, public culture collections such as the American Type Culture Collection, or from commercial suppliers.

Cells that can be used for production of the present chimeric proteins in vitro, ex vivo, and/or in vivo include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells (e.g., as obtained from bone marrow), umbilical cord blood, peripheral blood, fetal liver, etc. The choice of cell type depends on the type of the disease or disorder caused by or associated with inflammation of the integumentary system being treated or prevented, and can be determined by one of skill in the art.

Where necessary, the formulations comprising the chimeric protein, or the isolated polynucleotide or mmRNA (and/or additional agents) can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The formulations comprising the chimeric protein (and/or additional agents) of the present disclosure may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art)

In embodiments, any chimeric protein, or the isolated polynucleotide or mmRNA (and/or additional agents) described herein is formulated in accordance with routine procedures as a composition adapted for a mode of administration described herein.

Any chimeric protein, or the isolated polynucleotide or mmRNA (and/or additional agents) described herein can be administered orally. Such chimeric proteins (and/or additional agents) can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

The dosage of any chimeric protein, or the isolated polynucleotide or mmRNA (and/or additional agents) described herein as well as the dosing schedule can depend on various parameters, including, but not limited to, the disease being treated, the subject's general health, and the administering physician's discretion. Any chimeric protein described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional agent, to a subject in need thereof. In embodiments any chimeric protein and additional agent described herein are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days part, 4 days apart, 5 days apart, 6 days apart, 1 week apart, 2 weeks apart, 3 weeks apart, or 4 weeks apart.

The dosage of any chimeric protein, or the isolated polynucleotide or mmRNA (and/or additional agents) described herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In embodiments, delivery can be in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989).

Any chimeric protein, or the isolated polynucleotide or mmRNA (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In embodiments, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In embodiments, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Administration of any chimeric protein, or the isolated polynucleotide or mmRNA (and/or additional agents) described herein can, independently, be one to four times daily or one to four times per month or one to six times per year or once every two, three, four or five years. Administration can be for the duration of one day or one month, two months, three months, six months, one year, two years, three years, and may even be for the life of the subject.

The dosage regimen utilizing any chimeric protein, or the isolated polynucleotide or mmRNA (and/or additional agents) described herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the disclosure employed. Any chimeric protein (and/or additional agents) described herein can be administered in a single daily dose, or the total daily dosage can be administered in divided doses of two, three or four times daily. Furthermore, any chimeric protein (and/or additional agents) described herein can be administered continuously rather than intermittently throughout the dosage regimen.

In one aspect, the present disclosure provides a host cell comprising the polynucleotide of any of the embodiments disclosed herein.

In one aspect, the present disclosure provides a host cell comprising the vector of the embodiments disclosed herein. In one aspect, the present disclosure provides a host cell comprising an RNA (without limitations, e.g., mmRNA) encoring the chimeric protein of any of the embodiments disclosed herein.

Diseases/Disorders that may be Treated with the Chimeric Proteins or the Nucleic Acids Encoding the Chimeric Proteins of the Present Disclosure

In embodiments, the chimeric proteins disclosed herein or the nucleic acids encoding the chimeric proteins disclosed herein (without limitation e.g., modified mRNA (mmRNA)) are suitable for treating or preventing an autoimmune disease and/or allergic disease. In embodiments, the chimeric proteins disclosed herein or the nucleic acids encoding the chimeric proteins disclosed herein (without limitation e.g., mmRNA) are suitable for treating or preventing a disease or disorder selected from rheumatoid arthritis, juvenile idiopathic arthritis, psoriatic arthritis, ankylosing spondylitis, plaque psoriasis, Behcet's disease, sarcoidosis, wound ulcers, vasculitides, and pyoderma gangrenosum.

In embodiments, the chimeric proteins disclosed herein or the nucleic acids encoding the chimeric proteins disclosed herein (without limitation e.g., mmRNA) are suitable for treating or preventing the inflammation mediated by macrophages, monocytes, dendritic cells and/or T cells. In embodiments, the chimeric proteins disclosed herein or the nucleic acids encoding the chimeric proteins disclosed herein (without limitation e.g., mmRNA) are suitable for treating or preventing the inflammation mediated by cytokines, e.g., TNFα, IL-17, and/or IL-23. In embodiments, the inflammation is caused by or associated with a disease or disorder of the integumentary system. In embodiments, the inflammation is caused by or associated with a disease or disorder of the skin.

In embodiments, the chimeric proteins disclosed herein or the nucleic acids encoding the chimeric proteins disclosed herein (without limitation e.g., mmRNA) are suitable for treating or preventing in which TNFa activity is detrimental in a subject, including but not limited to rheumatoid arthritis, plaque psoriasis, psoriatic arthritis, polyarticular juvenile idiopathic arthritis (JIA), ankylosing spondylitis, Wegener's disease (granulomatosis), Crohn's disease (or inflammatory bowel disease), chronic obstructive pulmonary disease (COPD), hepatitis C, endometriosis, asthma, cachexia, psoriasis, or atopic dermatitis, or other inflammatory or autoimmune-related illness, disorder, or condition. Additional disorders that can be treated with the chimeric proteins disclosed herein or the nucleic acids encoding the chimeric proteins disclosed herein (without limitation e.g., mmRNA) are described in International Patent Application Publication Nos. WO 2000/062790; WO 2001/062272, and U.S. Pat. Nos. 8,772,458; and 7,700,318; and U.S. Patent Publication Nos. 2019/0135929, 2021/0177983, the entire contents of which are hereby incorporated by reference in their entirety.

In embodiments, the chimeric proteins disclosed herein or the nucleic acids encoding the chimeric proteins disclosed herein (without limitation e.g., mmRNA) are suitable for treating or preventing a disease or disorder of the skin selected from psoriasis, pemphigus vulgaris, scleroderma, atopic dermatitis, sarcoidosis, erythema nodosum, hidradenitis suppurativa, lichen planus, Sweet's syndrome, vitiligo, chronic paronychia, eczema, seborrheic dermatitis, and/or hives.

the chimeric proteins disclosed herein or the nucleic acids encoding the chimeric proteins disclosed herein (without limitation e.g., mmRNA) are suitable for treating or preventing psoriasis (without limitations, e.g., plaque psoriasis and psoriatic arthritis).

Psoriasis is a chronic inflammatory skin disease unique to humans. It is characterized by dense infiltration of T cells and cells of the innate immune system, including neutrophils, dendritic cells, macrophages, and NK cells in the skin lesions of psoriasis. Psoriasis is also characterized by hyperproliferation and abnormal differentiation of epithelial cells of the skin (e.g., keratinocytes), leading to a marked thickening of the epidermis. A dramatic increase in the number and size of blood vessels situated just below the epidermis is observed. Abscesses composed of neutrophils form within the epidermis, resulting in red, thickened, and flaking skin. The mechanisms which drive keratinocyte hyperproliferation and macrophage activation in the skin have not been fully defined. Psoriasis is a chronic, lifelong disease for many patients because the symptoms (e.g., the skin lesions) can be treated in the short term but the symptoms relapse when treatment is discontinued.

### Methods of Treatment

In one aspect, the present disclosure relates to a method for treating an autoimmune condition, or an inflammatory disorder subject comprising a step of administering to the subject a pharmaceutical composition comprising an isolated polynucleotide encoding a chimeric protein having a general structure of: N terminus (a)-(b)-(c)-C terminus, wherein: (a) is a first domain comprising an extracellular domain of tumor necrosis factor (TNF) receptor 2 (TNFR2), or a variant or a fragment thereof that is capable of binding a TNFR2 ligand, (c) is a second domain comprising an extracellular domain selected from CLEC7a, or a variant or a fragment thereof that capable of binding a CLEC7a ligand, DC-SIGN (CD209), or a variant or a fragment thereof that capable of binding a DC-SIGN (CD209) ligand, DECTIN2 (CLEC6A), or a variant or a fragment thereof that capable of binding a DECTIN2 (CLEC6A) ligand, Langerin (CD207,CLC4K), or a variant or a fragment thereof that capable of binding a Langerin (CD207, CLC4K) ligand, And CD69, or a variant or a fragment thereof that capable of binding a CD69 ligand, (b) is a linker adjoining the first and second domains, wherein the linker comprises at least one cysteine residue capable of forming a disulfide bond and/or comprises a hinge-C_{H2}-C_{H3} Fc domain. In embodiments, the TNFR2 ligand is TNFα. In embodiments, the CLEC7a ligand is a beta-1,3-linked and/or beta-1,6-linked glucan. In embodiments, the DC-SIGN (CD209) ligand is a Intercellular Adhesion Molecule 2 (ICAM2) and/or Intercellular Adhesion Molecule 3 (ICAM3). In embodiments, the DECTIN2 (CLEC6A) ligand is an alpha-mannan. In embodiments, the Langerin (CD207,CLC4K) ligand is a sulfated glycan, a mannosylated glycan, a keratan sulfate (KS) and/or a beta-glucan. In embodiments, the CD69 ligand is Galectin-1 (Gal-1) or the S100A8/S100A9 complex. In embodiments, the isolated polynucleotide is or comprises an mRNA. In embodiments, the isolated polynucleotide is or comprises an mRNA that is modified according to any of the embodiments disclosed herein.

In one aspect, the present disclosure relates to a method for inducing rapid and sustained immune inhibition subject comprising a step of administering to the subject a pharmaceutical composition comprising an isolated polynucleotide encoding a chimeric protein having a general structure of: N terminus-(a)-(b)-(c) -C terminus, wherein: (a) is a first domain comprising an extracellular domain of tumor necrosis factor (TNF) receptor 2 (TNFR2), or a variant or a fragment thereof that is capable of binding a TNFR2 ligand, (c) is a second domain comprising an extracellular domain selected from CLEC7a, or a variant or a fragment thereof that capable of binding a CLEC7a ligand, DC-SIGN (CD209), or a variant or a fragment thereof that capable of binding a DC-SIGN (CD209) ligand, DECTIN2 (CLEC6A), or a variant or a fragment thereof that capable of binding a DECTIN2 (CLEC6A) ligand, Langerin (CD207,CLC4K), or a variant or a fragment thereof that capable of binding a Langerin (CD207,CLC4K) ligand, And CD69, or a variant or a fragment thereof that capable of binding a CD69 ligand, (b) is a linker adjoining the first and second domains, wherein the linker comprises at least one cysteine residue capable of forming a disulfide bond and/or comprises a hinge-C_{H2}-C_{H3} Fc domain. In embodiments, the TNFR2 ligand is TNFα. In embodiments, the CLEC7a ligand is a beta-1,3-linked and/or beta-1,6-linked glucan. In embodiments, the DC-SIGN (CD209) ligand is a Intercellular Adhesion Molecule 2 (ICAM2) and/or Intercellular Adhesion Molecule 3 (ICAM3). In embodiments, the DECTIN2 (CLEC6A) ligand is an alpha-mannan. In embodiments, the Langerin (CD207,CLC4K) ligand is a sulfated glycan, a mannosylated glycan, a keratan sulfate (KS) and/or a beta-glucan. In embodiments, the CD69 ligand is Galectin-1 (Gal-1) or the S100A8/S100A9 complex. In embodiments, the isolated polynucleotide is or comprises an mRNA. In embodiments, the isolated polynucleotide is or comprises an mRNA that is modified according to any of the embodiments disclosed herein.

An aspect of the present invention is a method of treating an autoimmune disease comprising administering to a subject in need thereof an effective amount of a pharmaceutical composition comprising a pharmaceutically acceptable excipient or carrier, and a therapeutically effective amount of the chimeric protein of any of the embodiments disclosed herein, or the isolated polynucleotide encoding the chimeric protein of any of the embodiments disclosed herein (without limitation, e.g., mmRNA).

In one aspect, the present disclosure provides a method of treating or preventing an ailment caused by inflammation, the method comprising administering to a subject the chimeric protein of any of the embodiments disclosed herein, the isolated polynucleotide of any of the embodiments disclosed herein, the mmRNA of any of the embodiments disclosed herein, or the vector of any of the embodiments disclosed herein, or the host cell of any of the embodiments disclosed herein. In embodiments, the ailment is selected from psoriasis, psoriatic arthritis (PsA), plaque psoriasis, rheumatoid arthritis (RA), juvenile arthritis, ankylosing spondylitis, inflammatory bowel disease (IBD), ulcerative colitis (UC), and Crohn's disease.

In one aspect, the present disclosure provides a method of treating or preventing inflammation of the integumentary system, the method comprising administering to a subject the chimeric protein of any of the embodiments disclosed herein, the isolated polynucleotide of any of the embodiments disclosed herein, the mmRNA of any of the embodiments disclosed herein, or the vector of any of the embodiments disclosed herein, or the host cell of any of the embodiments disclosed herein.

In one aspect, the present disclosure provides a method of treating or preventing inflammation of the integumentary system, the method comprising administering to a subject the pharmaceutical composition of any of the embodiments disclosed herein.

In one aspect, the present disclosure provides a method of treating or preventing inflammation of the integumentary system, the method comprising administering to a subject the nucleic acid, e.g., the mmRNA of any of the embodiments disclosed herein.

In one aspect, the present disclosure provides a method of treating or preventing inflammation of the integumentary system, the method comprising administering to a subject an mmRNA encoding the chimeric protein of any of the embodiments disclosed herein.

In embodiments, the treatment reduces the levels of infiltration of T cells, neutrophils, dendritic cells, macrophages, and/or NK cells in the inflamed tissue compared to the levels of infiltration of T cells, neutrophils, dendritic cells, macrophages, and/or NK cells prior to the treatment. In embodiments, the treatment reduces the levels of TNFα, IL-17 and/or IL-23 in the inflamed tissue compared to the levels of TNFα, IL-17 and/or IL-23 prior to the treatment. In embodiments, the treatment reduces redness, thickening, and flaking of the skin compared to the reduces redness, thickening, and flaking of the skin prior to the treatment.

In one aspect, the present disclosure provides the pharmaceutical composition of any of the embodiments disclosed herein for use in treating or preventing an inflammation.

In one aspect, the present disclosure provides the chimeric protein of any of the embodiments disclosed herein, the isolated polynucleotide of any of the embodiments disclosed herein, the mmRNA of any of the embodiments disclosed herein, or the vector of any of the embodiments disclosed herein, the host cell of any of the embodiments disclosed herein for use in treating or preventing an inflammation.

In one aspect, the present disclosure provides a method of treating or preventing plaque psoriasis, the method comprising administering to a subject the pharmaceutical composition of any of the embodiments disclosed herein.

In one aspect, the present disclosure provides a method of treating or preventing plaque psoriasis, the method comprising administering to a subject the chimeric protein of any of the embodiments disclosed herein, and/or the isolated polynucleotide encoding the chimeric protein of any of the embodiments disclosed herein (without limitation, e.g., mmRNA).

In one aspect, the present disclosure provides a method of treating or preventing plaque psoriasis, the method comprising administering to a subject the pharmaceutical composition of any of the embodiments disclosed herein.

In one aspect, the present disclosure provides a method of treating or preventing plaque psoriasis, the method comprising administering to a subject the chimeric protein of any of the embodiments disclosed herein, and/or the isolated polynucleotide encoding the chimeric protein of any of the embodiments disclosed herein (without limitation, e.g., mmRNA).

In one aspect, the present disclosure provides a method of treating or preventing psoriatic arthritis, the method comprising administering to a subject the pharmaceutical composition of any of the embodiments disclosed herein.

In one aspect, the present disclosure provides a method of treating or preventing psoriatic arthritis, the method comprising administering to a subject the chimeric protein of any of the embodiments disclosed herein, and/or the isolated polynucleotide encoding the chimeric protein of any of the embodiments disclosed herein (without limitation, e.g., mmRNA).

In one aspect, the present disclosure provides a method of treating or preventing rheumatoid arthritis, the method comprising administering to a subject the pharmaceutical composition of any of the embodiments disclosed herein.

In one aspect, the present disclosure provides a method of treating or preventing rheumatoid arthritis, the method comprising administering to a subject the chimeric protein of any of the embodiments disclosed herein, and/or the isolated polynucleotide encoding the chimeric protein of any of the embodiments disclosed herein (without limitation, e.g., mmRNA).

In one aspect, the present disclosure provides a method of treating or preventing juvenile idiopathic arthritis, the method comprising administering to a subject the pharmaceutical composition of any of the embodiments disclosed herein.

In one aspect, the present disclosure provides a method of treating or preventing juvenile idiopathic arthritis, the method comprising administering to a subject the chimeric protein of any of the embodiments disclosed herein, and/or the isolated polynucleotide encoding the chimeric protein of any of the embodiments disclosed herein (without limitation, e.g., mmRNA).

In one aspect, the present disclosure provides a method of treating or preventing ankylosing spondylitis, the method comprising administering to a subject the pharmaceutical composition of any of the embodiments disclosed herein.

In one aspect, the present disclosure provides a method of treating or preventing ankylosing spondylitis, the method comprising administering to a subject the chimeric protein of any of the embodiments disclosed herein, and/or the isolated polynucleotide encoding the chimeric protein of any of the embodiments disclosed herein (without limitation, e.g., mmRNA).

In one aspect, the present disclosure provides a method of treating or preventing juvenile arthritis, the method comprising administering to a subject the chimeric protein of any of the embodiments disclosed herein, and/or the isolated polynucleotide encoding the chimeric protein of any of the embodiments disclosed herein (without limitation, e.g., mmRNA).

In one aspect, the present disclosure provides a method of treating or preventing inflammatory bowel disease (IBD), the method comprising administering to a subject the chimeric protein of any of the embodiments disclosed herein, and/or the isolated polynucleotide encoding the chimeric protein of any of the embodiments disclosed herein (without limitation, e.g., mmRNA).

In one aspect, the present disclosure provides a method of treating or preventing ulcerative colitis (UC), the method comprising administering to a subject the chimeric protein of any of the embodiments disclosed herein, and/or the isolated polynucleotide encoding the chimeric protein of any of the embodiments disclosed herein (without limitation, e.g., mmRNA).

In one aspect, the present disclosure provides a method of treating or preventing inflammatory Crohn's disease, the method comprising administering to a subject the chimeric protein of any of the embodiments disclosed herein, and/or the isolated polynucleotide encoding the chimeric protein of any of the embodiments disclosed herein (without limitation, e.g., mmRNA).

In one aspect, the present disclosure provides a method of treating or preventing an ailment caused by inflammation, the method comprising administering to a subject the chimeric protein of any of the embodiments disclosed herein, the isolated polynucleotide of any of the embodiments disclosed herein, the mmRNA of any of the embodiments disclosed herein, or the vector of any of the embodiments disclosed herein, or the host cell of any of the embodiments disclosed herein. In embodiments, the ailment is selected from psoriasis, psoriatic arthritis (PsA), plaque psoriasis, rheumatoid arthritis (RA), juvenile arthritis, ankylosing spondylitis, inflammatory bowel disease (IBD), ulcerative colitis (UC), Crohn's disease.

In embodiments, the method further comprises administering to the subject an anti-inflammatory drug. In embodiments, the anti-inflammatory drug is a non-steroidal anti-inflammatory or a corticosteroid.

In embodiments, the pharmaceutical composition and the anti-inflammatory drug are provided concurrently. In embodiments, the pharmaceutical composition and the anti-inflammatory drug are provided as two distinct pharmaceutical compositions. In embodiments, the pharmaceutical composition and the anti-inflammatory drug are provided as a single pharmaceutical composition. In embodiments, the pharmaceutical composition is provided after the anti-inflammatory drug is provided. In embodiments, the pharmaceutical composition is provided before the anti-inflammatory drug is provided.

In embodiments, the anti-inflammatory drug is a non-steroidal anti-inflammatory agent selected from acetyl salicylic acid (aspirin), benzyl-2,5-diacetoxybenzoic acid, celecoxib, diclofenac, etodolac, etofenamate, fulindac, glycol salicylate, ibuprofen, indomethacin, ketoprofen, methyl salicylate, nabumetone, naproxen, oxaprozin, phenylbutazone, piroxicam, salicylic acid, salicylmides, and vimovo® (a combination of naproxen and esomeprazole magnesium). In embodiments, the anti-inflammatory drug is a corticosteroid selected from alpha-methyl dexamethasone, amcinafel, amcinafide, beclomethasone dipropionate, beclomethasone dipropionate., betamethasone and the balance of its esters, betamethasone benzoate, betamethasone dipropionate, betamethasone valerate, beta-methyl betamethasone, bethamethasone, chloroprednisone, clescinolone, clobetasol valerate, clocortelone, cortisone, cortodoxone, desonide, desoxymethasone, dexamethasone, dichlorisone, diflorasone diacetate, diflucortolone valerate, difluorosone diacetate, difluprednate, fluadrenolone, flucetonide, fluclorolone acetonide, flucloronide, flucortine butylester, fludrocortisone, flumethasone pivalate, flunisolide, fluocinonide, fluocortolone, fluoromethalone, fluosinolone acetonide, fluperolone, fluprednidene (fluprednylidene) acetate, fluprednisolone, fluradrenolone acetonide, flurandrenolone, halcinonide, hydrocortisone, hydrocortisone acetate, hydrocortisone butyrate, hydroxyltriamcinolone, medrysone, meprednisone, methylprednisolone, paramethasone, prednisolone, prednisone, triamcinolone, and triamcinolone acetonide.

In embodiments, the method further comprises administering to the subject an immunosuppressive agent. In embodiments, the pharmaceutical composition and the immunosuppressive agent are provided concurrently. In embodiments, the pharmaceutical composition and the immunosuppressive agent are provided as two distinct pharmaceutical compositions. In embodiments, the pharmaceutical composition and the immunosuppressive agent are provided as a single pharmaceutical composition. In embodiments, the pharmaceutical composition is provided after the immunosuppressive agent is provided. In embodiments, the pharmaceutical composition is provided before the immunosuppressive agent is provided.

In embodiments, the immunosuppressive agent is selected from an antibody (e.g., basiliximab, daclizumab, and muromonab), an anti-immunophilin (e.g., cyclosporine, tacrolimus, and sirolimus), an antimetabolite (e.g., azathioprine and methotrexate), a cytostatic (such as alkylating agents), a cytotoxic antibiotic, an inteferon, a mycophenolate, an opioid, a small biological agent (e.g., fingolimod and myriocin), and a TNF binding protein.

In embodiments, the method further comprising administering to the subject an anti-inflammatory drug and an immunosuppressive agent. In embodiments, the pharmaceutical composition and the anti- inflammatory drug and the immunosuppressive agent are provided concurrently. In embodiments, the pharmaceutical composition and the anti-inflammatory drug and the immunosuppressive agent are provided as two distinct pharmaceutical compositions. In embodiments, the pharmaceutical composition and the anti-inflammatory drug and the immunosuppressive agent are provided as a single pharmaceutical composition. In embodiments, the pharmaceutical composition is provided after the anti-inflammatory drug and the immunosuppressive agent is provided. In embodiments, the pharmaceutical composition is provided before the anti-inflammatory drug and the immunosuppressive agent is provided.

In embodiments, the method further comprising administering to the subject a second pharmaceutical composition comprising an IL-12/IL-23 inhibitor and/or an IL-17 inhibitor. In embodiments, the pharmaceutical composition and the immunosuppressive agent are provided concurrently. In embodiments, the pharmaceutical composition and the immunosuppressive agent are provided as two distinct pharmaceutical compositions. In embodiments, the pharmaceutical composition and the immunosuppressive agent are provided as a single pharmaceutical composition. In embodiments, the pharmaceutical composition is provided after the immunosuppressive agent is provided. In embodiments, the pharmaceutical composition is provided before the immunosuppressive agent is provided. In embodiments, the IL-17 inhibitor is selected from secukinumab, ixekizumab, bimekizumab, and brodalumab. In embodiments, the IL12/IL-23 inhibitor is selected from utsekinumab, risankizumab, guselkumab, and tildrakizumab.

### Examples

The examples herein are provided to illustrate advantages and benefits of the present technology and to further assist a person of ordinary skill in the art with preparing or using the chimeric proteins and the nucleic acids encoding the same of the present technology. The examples herein are also presented in order to more fully illustrate the preferred aspects of the present technology. The examples should in no way be construed as limiting the scope of the present disclosure, as exemplified by the appended claims. The examples can include or incorporate any of the variations, aspects or embodiments of the present technology described above. The variations, aspects or embodiments described above may also further each include or incorporate the variations of any or all other variations, aspects or embodiments of the present technology.

### Example 1. Construction and Characterization of an Illustrative human TNFR2- and Clec7a-based Chimeric Protein

A construct encoding a human TNFR2- and human Clec7a-based chimeric protein was generated. The “human TNFR2-Fc-Clec7a” construct included the extracellular domain human TNFR2 fused to the extracellular domain of human Clec7a via a hinge-C_{H2}-C_{H3} Fc domain derived from IgG4.

The construct was codon optimized for expression in Chinese Hamster Ovary (CHO) cells, transfected into CHO cells and individual clones are selected for high expression. High expressing clones are then used for small-scale manufacturing in stirred bioreactors in serum-free media and the relevant chimeric fusion proteins are purified with Protein A binding resin columns.

The TNFR2-Fc-Clec7a construct was transiently expressed in 293 cells. The human TNFR2-Fc-Clec7a chimeric protein was purified using protein-A affinity chromatography and subjected to western blot analysis. Briefly, the purified human TNFR2-Fc-Clec7a protein was denatured and resolved using sodium dodecyl sulfate-polyacrylamide gel electrophoresis (SDS-PAGE) in triplicate. The protein was transferred on a membrane, and probed using an anti-human TNFR2 antibody, an anti-human-Fc antibody, or an anti-human

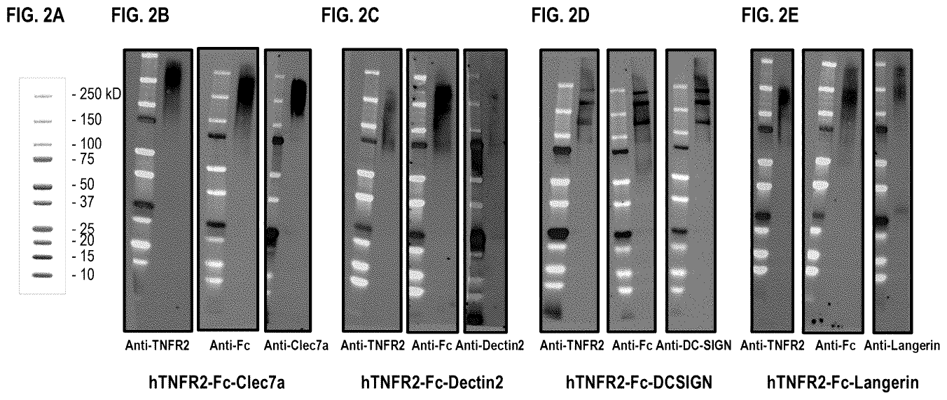

Clec7a antibody to confirm the presence of each domain of the TNFR2-Fc-Clec7a chimeric protein. The blots were compared with molecular weight ladder (FIG. 2A). As shown in FIG. 2B, the western blots exhibited a band of the TNFR2-Fc-Clec7a chimeric protein which was consistent with being a multimer. The detection of the band by each of the anti-human TNFR2 antibody, the anti-human-Fc antibody, or the anti-human Clec7a antibody indicated the existence of all three parts in the chimeric protein.

### Example 2. Construction and Characterization of an Illustrative human TNFR2- and Dectin2-based Chimeric Protein

A construct encoding a human TNFR2- and human Dectin2-based chimeric protein was generated. The “human TNFR2-Fc-Dectin2” construct included the extracellular domain human TNFR2 fused to the extracellular domain of human Dectin2 via a hinge-C_{H2}-C_{H3} Fc domain derived from IgG4.

The construct was codon optimized for expression in Chinese Hamster Ovary (CHO) cells, transfected into CHO cells and individual clones are selected for high expression. High expressing clones are then used for small-scale manufacturing in stirred bioreactors in serum-free media and the relevant chimeric fusion proteins are purified with Protein A binding resin columns.

The TNFR2-Fc-Dectin2 construct was transiently expressed in 293 cells. The human TNFR2-Fc-Dectin2 chimeric protein was purified using protein-A affinity chromatography and subjected to western blot analysis. Briefly, the purified human TNFR2-Fc-Dectin2 protein was denatured and resolved using sodium dodecyl sulfate-polyacrylamide gel electrophoresis (SDS-PAGE) in triplicate. The protein was transferred on a membrane, and probed using an anti-human TNFR2 antibody, an anti-human-Fc antibody, or an anti-human Dectin2 antibody to confirm the presence of each domain of the TNFR2-Fc-Dectin2 chimeric protein. The blots were compared with molecular weight ladder (FIG. 2A). As shown in FIG. 2C, the western blots exhibited a band of the TNFR2-Fc-Dectin2 chimeric protein which was consistent with being a multimer. The detection of the band by each of the anti-human TNFR2 antibody, the anti-human-Fc antibody, or the anti-human Dectin2 antibody indicated the existence of all three parts in the chimeric protein.

### Example 3. Construction and Characterization of an Illustrative human TNFR2- and DC-SIGN-based Chimeric Protein

A construct encoding a human TNFR2- and human DC-SIGN-based chimeric protein was generated. The “human TNFR2-Fc-DC-SIGN” construct included the extracellular domain human TNFR2 fused to the extracellular domain of human DC-SIGN via a hinge-C_{H2}-C_{H3} Fc domain derived from IgG4.

The construct was codon optimized for expression in Chinese Hamster Ovary (CHO) cells, transfected into CHO cells and individual clones are selected for high expression. High expressing clones are then used for small-scale manufacturing in stirred bioreactors in serum-free media and the relevant chimeric fusion proteins are purified with Protein A binding resin columns.

The TNFR2-Fc-DC-SIGN construct was transiently expressed in 293 cells. The human TNFR2-Fc-DC-SIGN chimeric protein was purified using protein-A affinity chromatography and subjected to western blot analysis. Briefly, the purified human TNFR2-Fc-DC-SIGN protein was denatured and resolved using sodium dodecyl sulfate-polyacrylamide gel electrophoresis (SDS-PAGE) in triplicate. The protein was transferred on a membrane, and probed using an anti-human TNFR2 antibody, an anti-human-Fc antibody, or an anti-human DC-SIGN antibody to confirm the presence of each domain of the TNFR2-Fc-DC-SIGN chimeric protein. The blots were compared with molecular weight ladder (FIG. 2A). As shown in FIG. 2D, the western blots exhibited a band of the TNFR2-Fc-DC-SIGN chimeric protein which was consistent with being a multimer. The detection of the band by each of the anti-human TNFR2 antibody, the anti-human-Fc antibody, or the anti-human DC-SIGN antibody indicated the existence of all three parts in the chimeric protein.

### Example 4. Construction and Characterization of an Illustrative human TNFR2- and Langerin-based Chimeric Protein

A construct encoding a human TNFR2- and human Langerin-based chimeric protein was generated. The “human TNFR2-Fc-Langerin” construct included the extracellular domain human TNFR2 fused to the extracellular domain of human Langerin via a hinge-C_{H2}-C_{H3} Fc domain derived from IgG4.

The construct was codon optimized for expression in Chinese Hamster Ovary (CHO) cells, transfected into CHO cells and individual clones are selected for high expression. High expressing clones are then used for small-scale manufacturing in stirred bioreactors in serum-free media and the relevant chimeric fusion proteins are purified with Protein A binding resin columns.

The TNFR2-Fc-Langerin construct was transiently expressed in 293 cells. The human TNFR2-Fc-Langerin chimeric protein was purified using protein-A affinity chromatography and subjected to western blot analysis. Briefly, the purified human TNFR2-Fc-Langerin protein was denatured and resolved using sodium dodecyl sulfate-polyacrylamide gel electrophoresis (SDS-PAGE) in triplicate. The protein was transferred on a membrane, and probed using an anti-human TNFR2 antibody, an anti-human-Fc antibody, or an anti-human Langerin antibody to confirm the presence of each domain of the TNFR2-Fc-Langerin chimeric protein. The blots were compared with molecular weight ladder (FIG. 2A). As shown in FIG. 2E, the western blots exhibited a band of the TNFR2-Fc-Langerin chimeric protein which was consistent with being a multimer. The detection of the band by each of the anti-human TNFR2 antibody, the anti-human-Fc antibody, or the anti-human Langerin antibody indicated the existence of all three parts in the chimeric protein.

### Example 5. The TNFR2- and C-Type-C-type Lectin Receptor (CLR)-Based Chimeric Proteins Disclosed Herein Bind to Their Respective Ligands

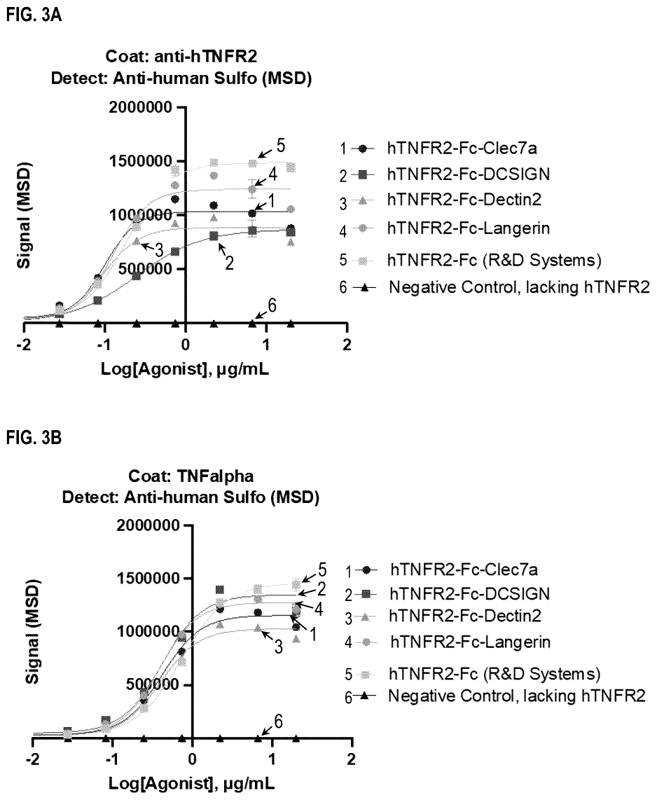

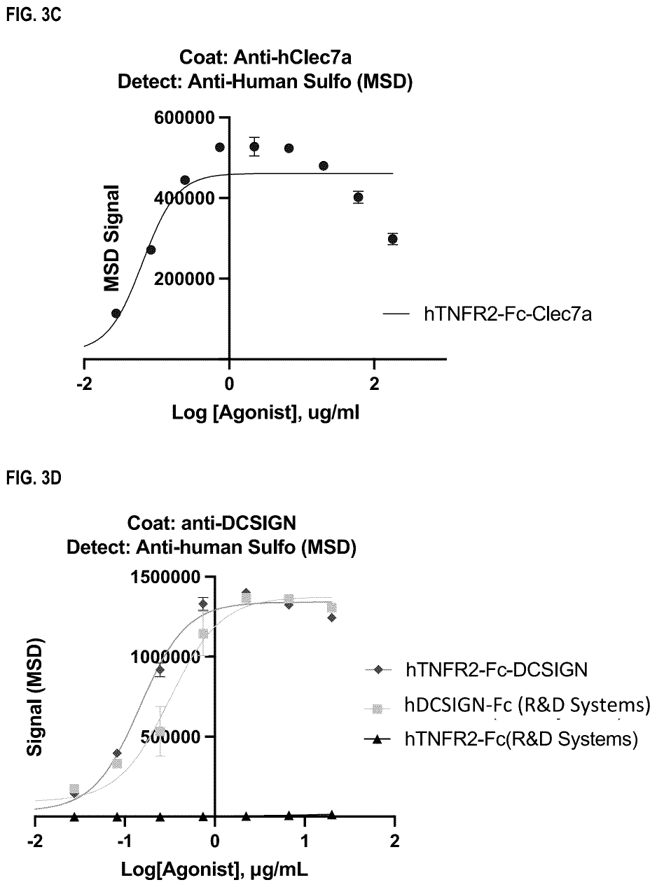

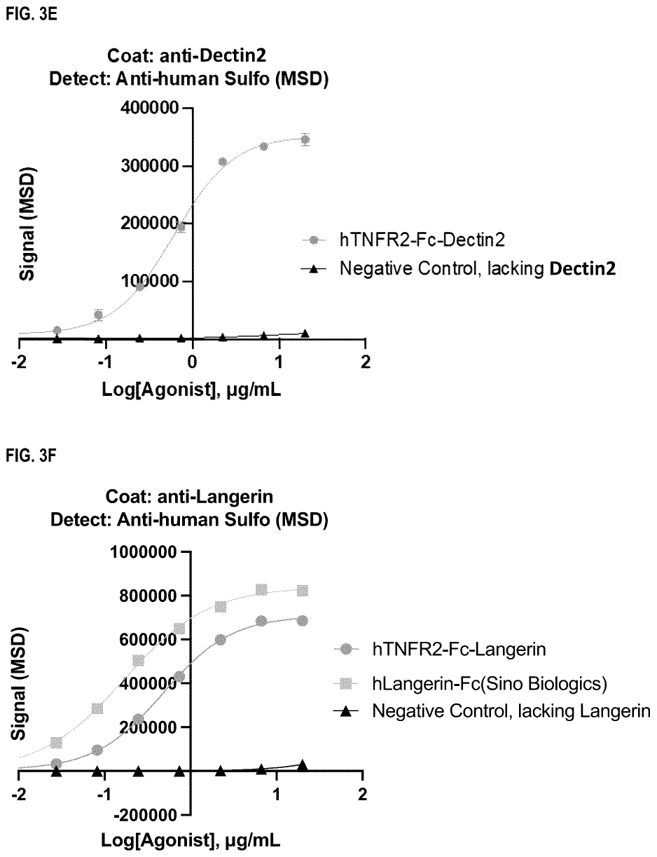

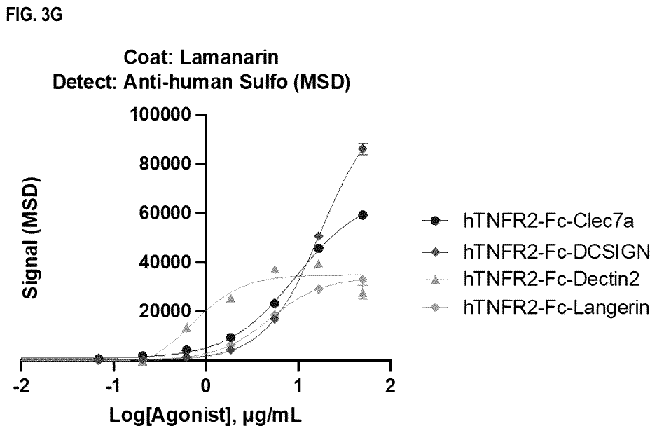

The binding of the chimeric proteins disclosed above to an anti-human TNFR2 antibody was measured using a Meso Scale Discovery (MSD) platform-based ELISA assay. Briefly, the anti-human TNFR2 antibody was coated on a plate. Increasing amounts of the human TNFR2-Fc-Clec7a, TNFR2-Fc-DC-SIGN, TNFR2-Fc-Dectin2, and TNFR2-Fc-Langerin chimeric proteins, a recombinant human TNFR2-Fc protein, or an irrelevant chimeric protein lacking any portion of TNFR2 were added to the plate for capture by the plate-bound the anti-human TNFR2 antibody. The proteins captured by the plate-bound anti-human TNFR2 antibody were detected using an anti-human Fc antibody and a SULFO-TAG conjugated secondary antibody. As shown in FIG. 3A, each of the human TNFR2-Fc-Clec7a, TNFR2-Fc-DC-SIGN, TNFR2-Fc-Dectin2, and TNFR2-Fc-Langerin chimeric proteins, and the recombinant human TNFR2-Fc protein bound to the anti-human TNFR2 antibody in a dose-dependent and saturable manner. In contrast, the irrelevant chimeric protein lacking any portion of TNFR2 showed no evidence of binding. These results indicate, inter alia, that the TNFR2- and C-type-C-type lectin receptor (CLR)-based chimeric proteins disclosed herein contemporaneously bind to a TNFR2 ligand and an Fc ligand.

The binding of the chimeric proteins disclosed above to TNFa was measured using a Meso Scale Discovery (MSD) platform-based ELISA assay. Briefly, TNFa was coated on a plate. Increasing amounts of the human TNFR2-Fc-Clec7a, TNFR2-Fc-DC-SIGN, TNFR2-Fc-Dectin2, and TNFR2-Fc-Langerin chimeric proteins, a recombinant human TNFR2-Fc protein, or an irrelevant chimeric protein lacking any portion of TNFR2 were added to the plate for capture by the plate-bound TNFα. The proteins captured by the plate-bound TNFα were detected using an anti-human Fc antibody and a SULFO-TAG conjugated secondary antibody. As shown in FIG. 3B, each of the human TNFR2-Fc-Clec7a, TNFR2-Fc-DC-SIGN, TNFR2-Fc-Dectin2, and TNFR2-Fc-Langerin chimeric proteins, and the recombinant human TNFR2-Fc protein bound to TNFa in a dose-dependent and saturable manner. In contrast, the irrelevant chimeric protein lacking any portion of TNFR2 showed no evidence of binding. These results indicate, inter alia, that the TNFR2- and C-type-C-type lectin receptor (CLR)-based chimeric proteins disclosed herein contemporaneously bind to the TNFR2 ligand TNFα and an Fc ligand.

The binding of the human TNFR2-Fc-Clec7a chimeric protein to an anti-human Clec7a antibody was measured using a Meso Scale Discovery (MSD) platform-based ELISA assay. Briefly, the anti-human Clec7a antibody was coated on a plate. Increasing amounts of the human TNFR2-Fc-Clec7a chimeric protein were added to the plate for capture by the plate-bound the anti-human Clec7a antibody. The protein captured by the plate-bound anti-human Clec7a antibody was detected using an anti-human Fc antibody and a SULFO-TAG conjugated secondary antibody. As shown in FIG. 3C, the human TNFR2-Fc-Clec7a chimeric protein bound to the anti-human Clec7a antibody in a dose-dependent and saturable manner. These results indicate, inter alia, that the human TNFR2-Fc-Clec7a chimeric protein contemporaneously binds to a Clec7a ligand and an Fc ligand.

The binding of the human TNFR2-Fc-DC-SIGN chimeric protein to an anti-human DC-SIGN antibody was measured using a Meso Scale Discovery (MSD) platform-based ELISA assay. Briefly, the anti-human DC-SIGN antibody was coated on a plate. Increasing amounts of the human TNFR2-Fc-DC-SIGN chimeric protein, recombinant human TNFR2-Fc fusion protein, and human DC-SIGN protein were added to the plate for capture by the plate-bound the anti-human DC-SIGN antibody. Human DC-SIGN-Fc and human TNFR2-Fc proteins were used as positive and negative controls, respectively. The protein captured by the plate-bound anti-human DC-SIGN antibody was detected using an anti-human Fc antibody and a SULFO-TAG conjugated secondary antibody. As shown in FIG. 3D, the human TNFR2-Fc-DC-SIGN chimeric protein bound to the anti-human DC-SIGN antibody in a dose-dependent and saturable manner. As expected, the recombinant DC-SIGN-Fc also bound to the anti-human DC-SIGN antibody in a dose-dependent and saturable manner but the human TNFR2-Fc protein did not bind the anti-human DC-SIGN antibody. These results indicate, inter alia, that the human TNFR2-Fc-DC-SIGN chimeric protein contemporaneously binds to a DC-SIGN ligand and an Fc ligand.

The binding of the human TNFR2-Fc-Dectin2 chimeric protein to an anti-human DC-SIGN antibody was measured using a Meso Scale Discovery (MSD) platform-based ELISA assay. Briefly, the anti-human DC-SIGN antibody was coated on a plate. Increasing amounts of the human TNFR2-Fc-Dectin2 chimeric protein, and an irrelevant chimeric protein lacking DC-SIGN were added to the plate for capture by the plate-bound the anti-human DC-SIGN antibody. The irrelevant chimeric protein was used as a negative control. The protein captured by the plate-bound anti-human DC-SIGN antibody was detected using an anti-human Fc antibody and a SULFO-TAG conjugated secondary antibody. As shown in FIG. 3E, the human TNFR2-Fc-Dectin2 chimeric protein bound to the anti-human Dectin2 antibody in a dose-dependent and saturable manner. As expected, the irrelevant chimeric protein lacking Dectin2 did not bind the anti-human Dectin2 antibody. These results indicate, inter alia, that the human TNFR2-Fc-Dectin2 chimeric protein contemporaneously binds to a Dectin2 ligand and an Fc ligand.

The binding of the human TNFR2-Fc-Langerin chimeric protein to an anti-human Langerin antibody was measured using a Meso Scale Discovery (MSD) platform-based ELISA assay. Briefly, the anti-human Langerin antibody was coated on a plate. Increasing amounts of the human TNFR2-Fc-Langerin chimeric protein, recombinant human Langerin-Fc fusion protein, and an irrelevant chimeric protein lacking Langerin were added to the plate for capture by the plate-bound the anti-human Langerin antibody. Human Langerin-Fc protein and human irrelevant chimeric protein were used as positive and negative controls, respectively. The protein captured by the plate-bound anti-human Langerin antibody was detected using an anti-human Fc antibody and a SULFO-TAG conjugated secondary antibody. As shown in FIG. 3F, the human TNFR2-Fc-Langerin chimeric protein bound to the anti-human Langerin antibody in a dose-dependent and saturable manner. As expected, the recombinant Langerin-Fc also bound to the anti-human Langerin antibody in a dose-dependent and saturable manner but the irrelevant chimeric protein did not bind the anti-human Langerin antibody. These results indicate, inter alia, that the human TNFR2-Fc-Langerin chimeric protein contemporaneously binds to a Langerin ligand and an Fc ligand.

Laminarin, which is a water-soluble polysaccharide that consists of β-(1-3)-glucan with β-(1-6)-linkages, is extracted and isolated from the dry thallus of brown seaweeds like Laminaria japonica, Ecklonia kurome, or Eisenia bicyclis. Many C-type lectins bind Laminarin. The binding of the chimeric proteins disclosed above to Laminarin was measured using a Meso Scale Discovery (MSD) platform-based ELISA assay. Briefly, Laminarin was coated on a plate. Increasing amounts of the human TNFR2-Fc-Clec7a, TNFR2-Fc-DC-SIGN, TNFR2-Fc-Dectin2, and TNFR2-Fc-Langerin chimeric proteins were added to the plate for capture by the plate-bound Laminarin. The proteins captured by the plate-bound Laminarin were detected using an anti-human Fc antibody and a SULFO-TAG conjugated secondary antibody. As shown in FIG. 3G, each of the human TNFR2-Fc-Clec7a, TNFR2-Fc-DC-SIGN, TNFR2-Fc-Dectin2, and TNER2-Fc-Langerin chimeric proteins, and the recombinant human TNFR2-Fc protein bound to Laminarin in a dose-dependent and saturable manner, with differing kinetics. These results indicate, inter alia, that the TNFR2- and C-type-C-type lectin receptor (CLR)-based chimeric proteins disclosed herein bind to Laminarin with differing affinities.

### Example 6. Construction and Characterization of Mouse Surrogates of the TNFR2- and C-Type-C-type Lectin Receptor (CLR)-Based Chimeric Proteins Disclosed Herein

Mouse surrogate of the human TNFR2- and C-Type-C-type lectin receptor (CLR)-based chimeric proteins TNFR2-Fc-Clec7a chimeric protein was constructed for its use in mouse models of disease.

A construct encoding a TNFR2- and Clec7a-based chimeric protein was generated. The construct included the extracellular domain TNFR2 fused to the extracellular domain of Clec7a via a hinge-C_{H2}-C_{H3} Fc domain derived from IgG. The construct was codon optimized for expression in Chinese Hamster Ovary (CHO) cells, transfected into CHO cells and individual clones are selected for high expression. High expressing clones are then used for small-scale manufacturing in stirred bioreactors in serum-free media and the relevant chimeric fusion proteins are purified with Protein A binding resin columns.

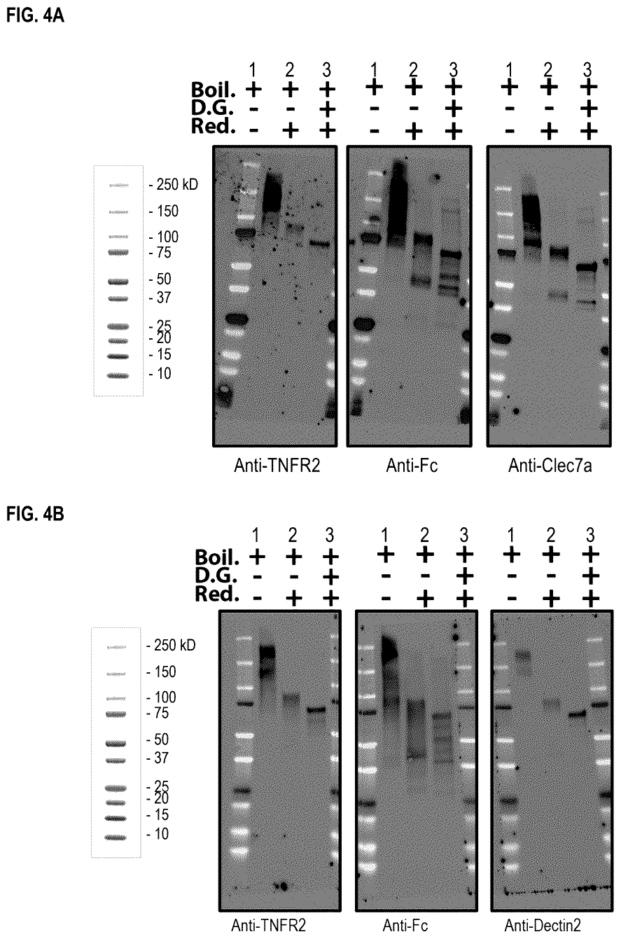

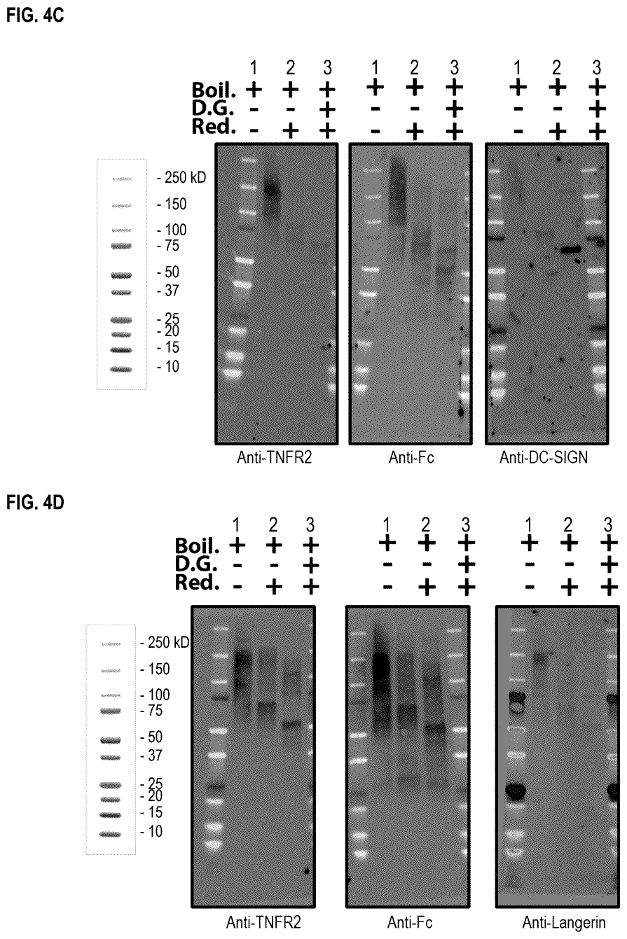

The TNFR2-Fc-Clec7a construct was transiently expressed in 293 cells. The TNFR2-Fc-Clec7a construct was transiently expressed in 293 cells and purified using protein-A affinity chromatography. To understand the native structure of the TNFR2-Fc-Clec7a chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the TNFR2-Fc-Clec7a chimeric protein, the gels were run in triplicates and probed using an anti-TNFR2 antibody (FIG. 4A, left blot), an anti-Fc antibody (FIG. 4A, center blot), or an anti-Clec7a antibody (FIG. 4A, right blot). The Western blots indicated the presence of a dominant dimer band in the non-reduced lanes (FIG. 4A, lane 1 in each blot), which was reduced to a glycosylated monomeric band in the presence of the reducing agent, β-mercaptoethanol (FIG. 4A, lane 2 in each blot). As shown in FIG. 4A, lane 3 in each blot, the chimeric protein ran as a monomer at the predicted molecular weight in the presence of both a reducing agent (β-mercaptoethanol) and a deglycosylation agent.

A construct encoding a TNFR2- and Dectin2-based chimeric protein was generated. The construct included the extracellular domain TNFR2 fused to the extracellular domain of Dectin2 via a hinge-C_{H2}-C_{H3} Fc domain derived from IgG. The construct was codon optimized for expression in Chinese Hamster Ovary (CHO) cells, transfected into CHO cells and individual clones are selected for high expression. High expressing clones are then used for small-scale manufacturing in stirred bioreactors in serum-free media and the relevant chimeric fusion proteins are purified with Protein A binding resin columns.

The TNFR2-Fc-Dectin2 construct was transiently expressed in 293 cells. The TNFR2-Fc-Dectin2 construct was transiently expressed in 293 cells and purified using protein-A affinity chromatography. To understand the native structure of the TNFR2-Fc-Dectin2 chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the TNFR2-Fc-Dectin2 chimeric protein, the gels were run in triplicates and probed using an anti-TNFR2 antibody (FIG. 4B, left blot), an anti-Fc antibody (FIG. 4B, center blot), or an anti-Dectin2 antibody (FIG. 4B, right blot). The Western blots indicated the presence of a dominant dimer band in the non-reduced lanes (FIG. 4B, lane 1 in each blot), which was reduced to a glycosylated monomeric band in the presence of the reducing agent, β-mercaptoethanol (FIG. 4B, lane 2 in each blot). As shown in FIG. 4B, lane 3 in each blot, the chimeric protein ran as a monomer at the predicted molecular weight in the presence of both a reducing agent (β-mercaptoethanol) and a deglycosylation agent.

A construct encoding a TNFR2- and DC-SIGN-based chimeric protein was generated. The construct included the extracellular domain TNFR2 fused to the extracellular domain of DC-SIGN via a hinge-C_{H2}-C_{H3} Fc domain derived from IgG. The construct was codon optimized for expression in Chinese Hamster Ovary (CHO) cells, transfected into CHO cells and individual clones are selected for high expression. High expressing clones are then used for small-scale manufacturing in stirred bioreactors in serum-free media and the relevant chimeric fusion proteins are purified with Protein A binding resin columns.

The TNFR2-Fc-DC-SIGN construct was transiently expressed in 293 cells. The TNFR2-Fc-DC-SIGN construct was transiently expressed in 293 cells and purified using protein-A affinity chromatography. To understand the native structure of the TNFR2-Fc-DC-SIGN chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with-mercaptoethanol and a deglycosylation agent, and boiled in the presence of SDS). In addition, to confirm the presence of each domain of the TNFR2-Fc-DC-SIGN chimeric protein, the gels were run in triplicates and probed using an anti-TNFR2 antibody (FIG. 4C, left blot), an anti-Fc antibody (FIG. 4C, center blot), or an anti-DC-SIGN antibody (FIG. 4C, right blot). The Western blots indicated the presence of a dominant dimer band in the non-reduced lanes (FIG. 4C, lane 1 in each blot), which was reduced to a glycosylated monomeric band in the presence of the reducing agent, β-mercaptoethanol (FIG. 4C, lane 2 in each blot). As shown in FIG. 4C, lane 3 in each blot, the chimeric protein ran as a monomer at the predicted molecular weight in the presence of both a reducing agent (β-mercaptoethanol) and a deglycosylation agent.

A construct encoding a TNFR2- and Langerin-based chimeric protein was generated. The construct included the extracellular domain TNFR2 fused to the extracellular domain of Langerin via a hinge-C_{H2}-C_{H3} Fc domain derived from IgG. The construct was codon optimized for expression in Chinese Hamster Ovary (CHO) cells, transfected into CHO cells and individual clones are selected for high expression. High expressing clones are then used for small-scale manufacturing in stirred bioreactors in serum-free media and the relevant chimeric fusion proteins are purified with Protein A binding resin columns.

The TNFR2-Fc-Langerin construct was transiently expressed in 293 cells. The TNFR2-Fc-Langerin construct was transiently expressed in 293 cells and purified using protein-A affinity chromatography. To understand the native structure of the TNFR2-Fc-Langerin chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the TNFR2-Fc-Langerin chimeric protein, the gels were run in triplicates and probed using an anti-TNFR2 antibody (FIG. 4D, left blot), an anti-Fc antibody (FIG. 4D, center blot), or an anti-Langerin antibody (FIG. 4D, right blot). The Western blots indicated the presence of a dominant dimer band in the non-reduced lanes (FIG. 4D, lane 1 in each blot), which was reduced to a glycosylated monomeric band in the presence of the reducing agent, β-mercaptoethanol (FIG. 4D, lane 2 in each blot). As shown in FIG. 4D, lane 3 in each blot, the chimeric protein ran as a monomer at the predicted molecular weight in the presence of both a reducing agent (β-mercaptoethanol) and a deglycosylation agent.

### Example 7. The TNFR2- and C-Type-C-type Lectin Receptor (CLR)-Based Chimeric Proteins Disclosed Herein Bind to Their Respective Ligands

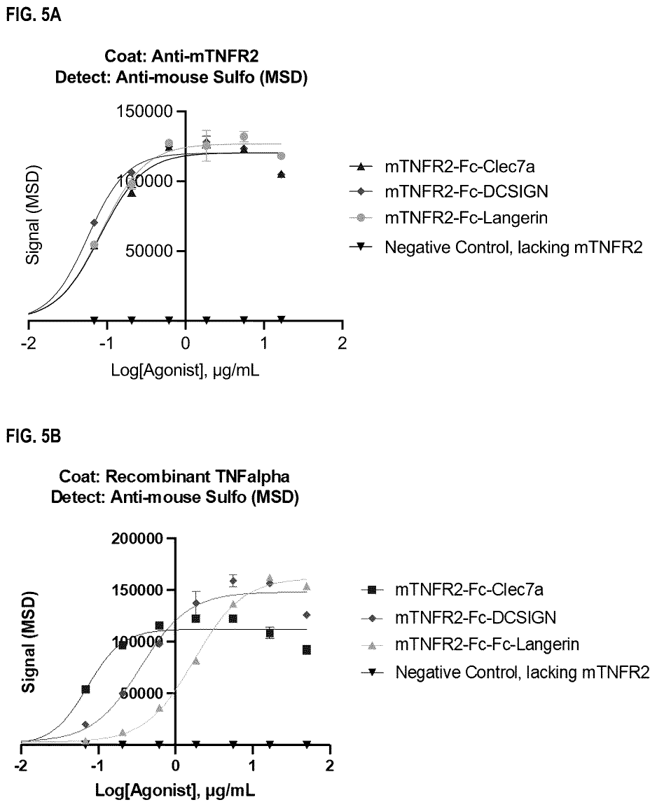

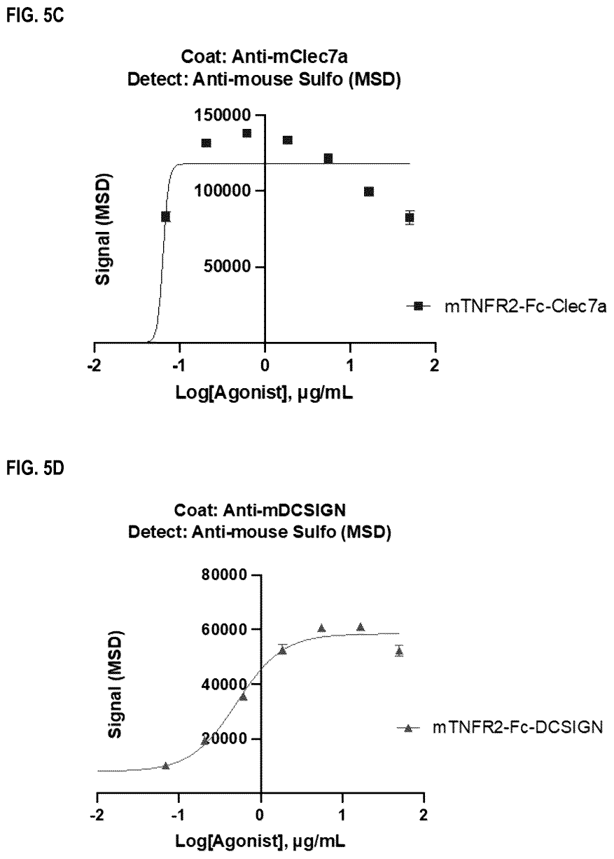

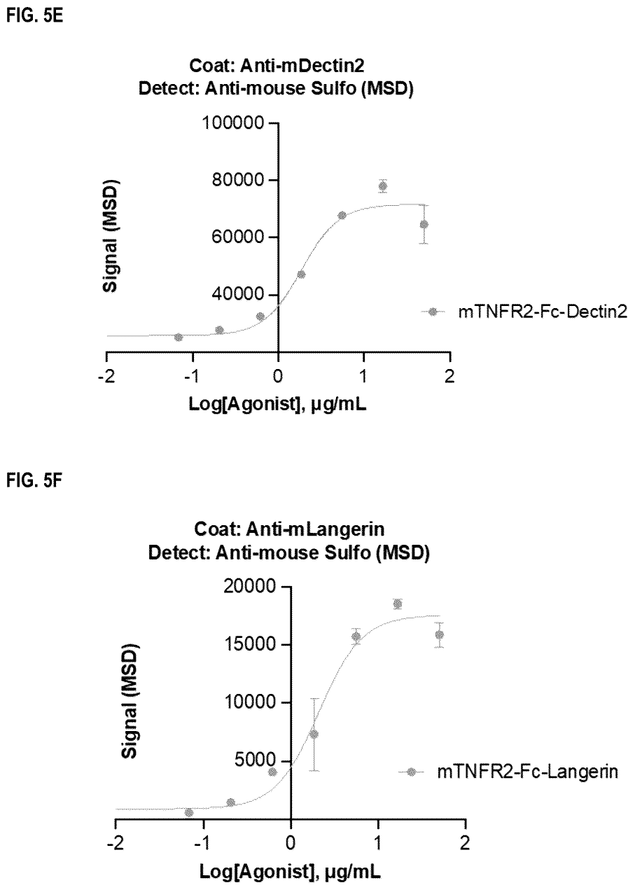

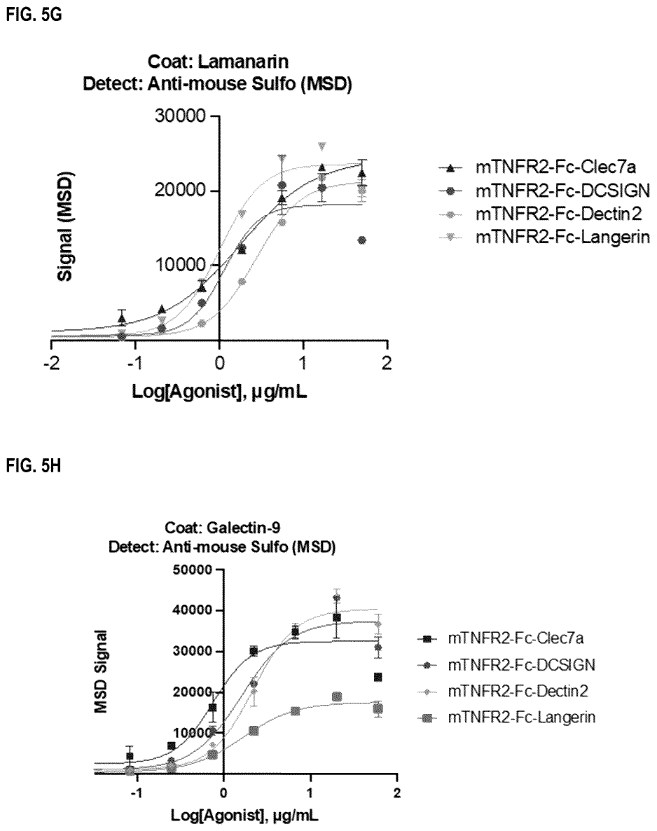

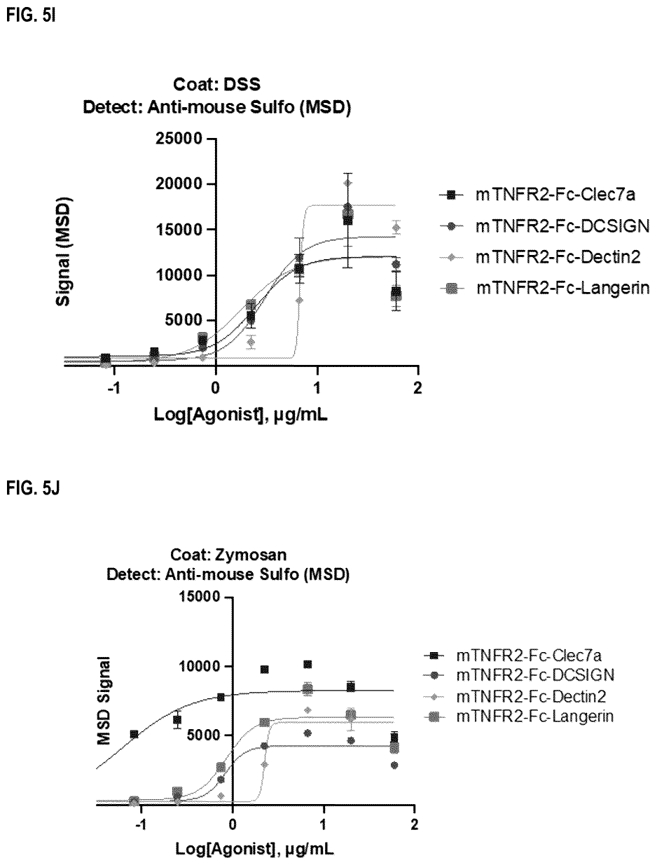

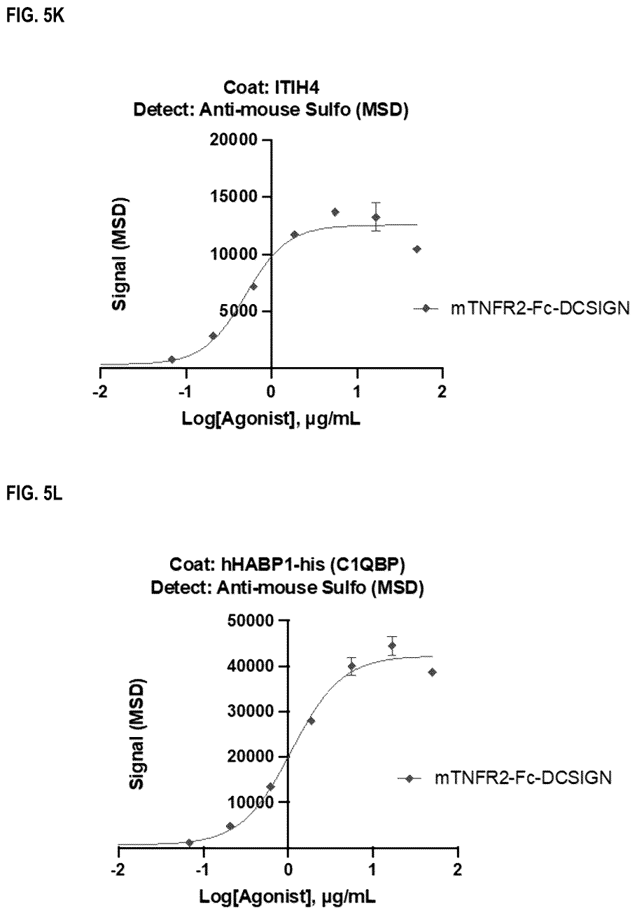

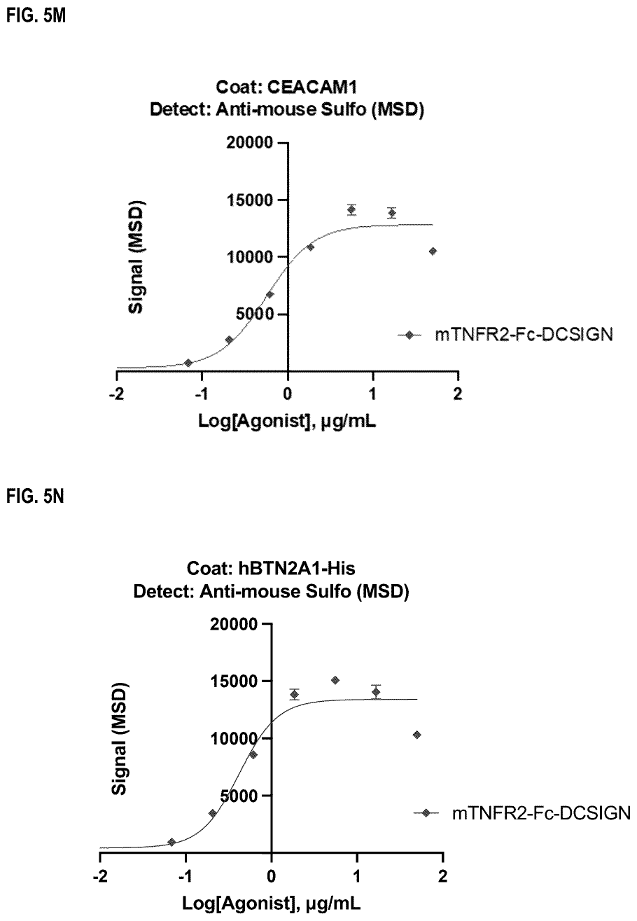

The binding of the chimeric proteins disclosed above to an anti-mouse TNFR2 antibody was measured using a Meso Scale Discovery (MSD) platform-based ELISA assay. Briefly, the anti-TNFR2 antibody was coated on a plate. Increasing amounts of the TNFR2-Fc-Clec7a, TNFR2-Fc-DC-SIGN, TNFR2-Fc-Langerin, or an irrelevant chimeric protein lacking any portion of mouse TNFR2 were added to the plate for capture by the plate-bound the anti-TNFR2 antibody. The proteins captured by the plate-bound anti-TNFR2 antibody were detected using a SULFO-TAG conjugated anti-mouse antibody. As shown in FIG. 5A, each of the mouse TNFR2-Fc-Clec7a, TNFR2-Fc-DC-SIGN, and TNFR2-Fc-Langerin chimeric proteins to the anti-TNFR2 antibody in a dose-dependent and saturable manner. In contrast, the irrelevant chimeric protein lacking any portion of TNFR2 showed no evidence of binding. These results indicate, inter alia, that the mouse TNFR2- and C-type-C-type lectin receptor (CLR)-based chimeric proteins disclosed herein contemporaneously bind to a TNFR2 ligand and an Fc ligand.

The binding of the chimeric proteins disclosed above to recombinant TNFα was measured using a Meso Scale Discovery (MSD) platform-based ELISA assay. Briefly, recombinant TNFα was coated on a plate. Increasing amounts of the TNFR2-Fc-Clec7a, TNFR2-Fc-DC-SIGN, TNFR2-Fc-Langerin, or an irrelevant chimeric protein lacking any portion of mouse TNFR2 were added to the plate for capture by the plate-bound the recombinant TNFα. The proteins captured by the plate-bound recombinant TNFα were detected using a SULFO-TAG conjugated anti-mouse antibody. As shown in FIG. 5B, each of the mouse TNFR2-Fc-Clec7a, TNFR2-Fc-DC-SIGN, and TNFR2-Fc-Langerin chimeric proteins to the recombinant TNFα in a dose-dependent and saturable manner. In contrast, the irrelevant chimeric protein lacking any portion of TNFR2 showed no evidence of binding. These results indicate, inter alia, that the mouse TNFR2- and C-type-C-type lectin receptor (CLR)-based chimeric proteins disclosed herein contemporaneously bind to TNFα and an Fc ligand.

The binding of the mouse TNFR2-Fc-Clec7a chimeric protein to an anti-mouse Clec7a antibody was measured using a Meso Scale Discovery (MSD) platform-based ELISA assay. Briefly, the anti-Clec7a antibody was coated on a plate. Increasing amounts of the TNFR2-Fc-Clec7a chimeric protein were added to the plate for capture by the plate-bound the anti-Clec7a antibody. The protein captured by the plate-bound anti-Clec7a antibody was detected using an anti-Fc antibody and a SULFO-TAG conjugated secondary antibody. As shown in FIG. 5C, the TNFR2-Fc-Clec7a chimeric protein bound to the anti-Clec7a antibody. These results indicate, inter alia, that the TNFR2-Fc-Clec7a chimeric protein contemporaneously binds to a Clec7a ligand and an Fc ligand.

The binding of the mouse TNFR2-Fc-DC-SIGN chimeric protein to an anti-mouse DC-SIGN antibody was measured using a Meso Scale Discovery (MSD) platform-based ELISA assay. Briefly, the anti-DC-SIGN antibody was coated on a plate. Increasing amounts of the TNFR2-Fc-DC-SIGN chimeric protein were added to the plate for capture by the plate-bound the anti-DC-SIGN antibody. The protein captured by the plate-bound anti-DC-SIGN antibody was detected using an anti-Fc antibody and a SULFO-TAG conjugated secondary antibody. As shown in FIG. 5D, the TNFR2-Fc-DC-SIGN chimeric protein bound to the anti-DC-SIGN antibody in a dose-dependent and saturable manner. These results indicate, inter alia, that the TNFR2-Fc-DC-SIGN chimeric protein contemporaneously binds to a DC-SIGN ligand and an Fc ligand.

The binding of the mouse TNFR2-Fc-Dectin2 chimeric protein to an anti-mouse Dectin2 antibody was measured using a Meso Scale Discovery (MSD) platform-based ELISA assay. Briefly, the anti-Dectin2 antibody was coated on a plate. Increasing amounts of the TNFR2-Fc-Dectin2 chimeric protein were added to the plate for capture by the plate-bound the anti-Dectin2 antibody. The protein captured by the plate-bound anti-Dectin2 antibody was detected using an anti-Fc antibody and a SULFO-TAG conjugated secondary antibody. As shown in FIG. 5E, the TNFR2-Fc-Dectin2 chimeric protein bound to the anti-Dectin2 antibody in a dose-dependent and saturable manner. These results indicate, inter alia, that the TNFR2-Fc-Dectin2 chimeric protein contemporaneously binds to a Dectin2 ligand and an Fc ligand.

The binding of the mouse TNFR2-Fc-Langerin chimeric protein to an anti-mouse Langerin antibody was measured using a Meso Scale Discovery (MSD) platform-based ELISA assay. Briefly, the anti-Langerin antibody was coated on a plate. Increasing amounts of the TNFR2-Fc-Langerin chimeric protein were added to the plate for capture by the plate-bound the anti-Langerin antibody. The protein captured by the plate-bound anti-Langerin antibody was detected using an anti-Fc antibody and a SULFO-TAG conjugated secondary antibody. As shown in FIG. 5F, the TNFR2-Fc-Langerin chimeric protein bound to the anti-Langerin antibody in a dose-dependent and saturable manner. These results indicate, inter alia, that the TNFR2-Fc-Langerin chimeric protein contemporaneously binds to a Langerin ligand and an Fc ligand.

The binding of the mouse TNFR2-Fc-Clec7a, TNFR2-Fc-DC-SIGN, TNFR2-Fc-Dectin2, and TNER2-Fc-Langerin chimeric proteins to Laminarin, a water-soluble polysaccharide that consists of β-(1-3)-glucan with β-(1-6)-linkages, was measured using a Meso Scale Discovery (MSD) platform-based ELISA assay. Briefly, Laminarin was coated on a plate. Increasing amounts of the mouse TNFR2-Fc-Clec7a, TNFR2-Fc-DC-SIGN, TNFR2-Fc-Dectin2, and TNER2-Fc-Langerin chimeric proteins were added to the plate for capture by the plate-bound Laminarin. The proteins captured by the plate-bound Laminarin were detected using a SULFO-TAG conjugated anti-mouse antibody. As shown in FIG. 5G, each of the mouse TNFR2-Fc-Clec7a, TNFR2-Fc-DC-SIGN, TNFR2-Fc-Dectin2, and TNFR2-Fc-Langerin chimeric proteins bound to Laminarin in a dose-dependent and saturable manner, with differing kinetics. These results indicate, inter alia, that the mouse surrogate chimeric proteins disclosed herein bind to Laminarin with differing affinities.

Galectin-9, which is a β-galactoside-binding lectin capable of promoting or suppressing the progression of infectious diseases, is known to interact with. C-type lectin receptor within both the human and murine dendritic cell cytosol. Cano et al., Intracellular Galectin-9 Controls Dendritic Cell Function by Maintaining Plasma Membrane Rigidity, iScience. 2019; 22:240-255. The binding of the mouse TNFR2-Fc-Clec7a, TNFR2-Fc-DC-SIGN, TNFR2-Fc-Dectin2, and TNFR2-Fc-Langerin chimeric proteins to Galectin-9 was measured using a Meso Scale Discovery (MSD) platform-based ELISA assay. Briefly, Galectin-9 was coated on a plate. Increasing amounts of the mouse TNFR2-Fc-Clec7a, TNFR2-Fc-DC-SIGN, TNFR2-Fc-Dectin2, and TNFR2-Fc-Langerin chimeric proteins were added to the plate for capture by the plate-bound Galectin-9. The proteins captured by the plate-bound Galectin-9 were detected using a SULFO-TAG conjugated anti-mouse antibody. As shown in FIG. 5H, each of the mouse TNFR2-Fc-Clec7a, TNFR2-Fc-DC-SIGN, TNFR2-Fc-Dectin2, and TNFR2-Fc-Langerin chimeric proteins bound to Galectin-9 in a dose-dependent and saturable manner, with differing kinetics. These results indicate, inter alia, that the mouse surrogate chimeric proteins disclosed herein bind to Galectin-9 with differing affinities.

The binding of the mouse TNFR2-Fc-Clec7a, TNFR2-Fc-DC-SIGN, TNFR2-Fc-Dectin2, and TNFR2-Fc-Langerin chimeric proteins dextran sulphate sodium (DSS) was measured using a Meso Scale Discovery (MSD) platform-based ELISA assay. Briefly, DSS was coated on a plate. Increasing amounts of the mouse TNFR2-Fc-Clec7a, TNFR2-Fc-DC-SIGN, TNFR2-Fc-Dectin2, and TNFR2-Fc-Langerin chimeric proteins were added to the plate for capture by the plate-bound DSS. The proteins captured by the plate-bound DSS were detected using a SULFO-TAG conjugated anti-mouse antibody. As shown in FIG. 5I, each of the mouse TNFR2-Fc-Clec7a, TNFR2-Fc-DC-SIGN, TNFR2-Fc-Dectin2, and TNER2-Fc-Langerin chimeric proteins bound to DSS in a dose-dependent and saturable manner, with differing kinetics. These results indicate, inter alia, that the mouse surrogate chimeric proteins disclosed herein bind to DSS with differing affinities.

The yeast cell wall (zymosan) binds the C-type lectin receptor Dectin-1 and induces the recruitment of the protein tyrosine kinase Syk, which in turn induces cytokine production. Rogers et al., Syk-Dependent Cytokine Induction by Dectin-1 Reveals a Novel Pattern Recognition Pathway for C Type Lectins, Immunity, 2005; 22 507-517. The binding of the mouse TNFR2-Fc-Clec7a, TNFR2-Fc-DC-SIGN, TNFR2-Fc-Dectin2, and TNFR2-Fc-Langerin chimeric proteins to zymosan was measured using a Meso Scale Discovery (MSD) platform-based ELISA assay. Briefly, zymosan was coated on a plate. Increasing amounts of the mouse TNFR2-Fc-Clec7a, TNFR2-Fc-DC-SIGN, TNFR2-Fc-Dectin2, and TNER2-Fc-Langerin chimeric proteins were added to the plate for capture by the plate-bound zymosan. The proteins captured by the plate-bound zymosan were detected using a SULFO-TAG conjugated anti-mouse antibody. As shown in FIG. 5J, each of the mouse TNFR2-Fc-Clec7a, TNFR2-Fc-DC-SIGN, TNFR2-Fc-Dectin2, and TNFR2-Fc-Langerin chimeric proteins bound to zymosan in a dose-dependent and saturable manner, with differing kinetics. These results indicate, inter alia, that the mouse surrogate chimeric proteins disclosed herein bind to zymosan with differing affinities.

The binding of the mouse TNFR2-Fc-DC-SIGN chimeric proteins to Inter-α-inhibitor heavy chain 4 (ITIH4) was measured using a Meso Scale Discovery (MSD) platform-based ELISA assay. Briefly, ITIH4 was coated on a plate. Increasing amounts of the mouse TNFR2-Fc-DC-SIGN chimeric protein was added to the plate for capture by the plate-bound ITIH4. The protein captured by the plate-bound ITIH4 were detected using a SULFO-TAG conjugated anti-mouse antibody. As shown in FIG. 5K, the mouse TNFR2-Fc-DC-SIGN chimeric protein bound to ITIH4 in a dose-dependent and saturable manner.

The binding of the mouse TNFR2-Fc-DC-SIGN chimeric proteins to hyaluronan binding protein 1 (HABP1) was measured using a Meso Scale Discovery (MSD) platform-based ELISA assay. Briefly, HABP1 was coated on a plate. Increasing amounts of the mouse TNFR2-Fc-DC-SIGN chimeric protein was added to the plate for capture by the plate-bound HABP1. The protein captured by the plate-bound HABP1 were detected using a SULFO-TAG conjugated anti-mouse antibody. As shown in FIG. 5L, the mouse TNFR2-Fc-DC-SIGN chimeric protein bound to HABP1 in a dose-dependent and saturable manner.

The binding of the mouse TNFR2-Fc-DC-SIGN chimeric proteins to carcinoembryonic antigen-related cell adhesion molecule 1 (CEACAM1) was measured using a Meso Scale Discovery (MSD) platform-based ELISA assay. Briefly, CAECAM1 was coated on a plate. Increasing amounts of the mouse TNFR2-Fc-DC-SIGN chimeric protein was added to the plate for capture by the plate-bound CAECAM1. The protein captured by the plate-bound CAECAM1 were detected using a SULFO-TAG conjugated anti-mouse antibody. As shown in FIG. 5M, the mouse TNFR2-Fc-DC-SIGN chimeric protein bound to CAECAM1 in a dose-dependent and saturable manner.

The binding of the mouse TNFR2-Fc-DC-SIGN chimeric proteins to Butyrophilin subfamily 2 member A1 (BTN2A1) was measured using a Meso Scale Discovery (MSD) platform-based ELISA assay. Briefly, BTN2A1 was coated on a plate. Increasing amounts of the mouse TNFR2-Fc-DC-SIGN chimeric protein was added to the plate for capture by the plate-bound BTN2A1. The proteins captured by the plate-bound BTN2A1 were detected using a SULFO-TAG conjugated anti-mouse antibody. As shown in FIG. 5N, the mouse TNFR2-Fc-DC-SIGN chimeric protein bound to BTN2A1 in a dose-dependent and saturable manner.

### Example 8. Synthesis of Modified mRNA Encoding the Chimeric Proteins Disclosed Herein

A modified mRNA encoding the TNFR2-Fc-Clec7a, TNFR2-Fc-langerin, TNFR2-Fc-DC-SIGN, and TNFR2-Fc-Dectin-2 chimeric proteins and their mouse surrogates was synthesized. Briefly, genes encoding the chimeric proteins were designed, codon optimized for expression in human cells. The genes comprising open reading frames for the TNFR2-Fc-Clec7a, TNFR2-Fc-langerin, TNFR2-Fc-DC-SIGN, and TNFR2-Fc-Dectin-2 chimeric proteins also included 5′ cap and 3′ poly-A tail. Modifications of the mRNA were then designed. Synthesis was performed using any of the methods known in art. For example, modified mRNA can be synthesized by chemical synthesis or by in vitro transcription using mutant RNA polymerases that incorporate modified nucleotides in mRNA.

The modified mRNA was transfected in cells in vitro and the expression of the TNFR2-Fc-Clec7a, TNFR2-Fc-langerin, TNFR2-Fc-DC-SIGN, and TNFR2-Fc-Dectin-2 chimeric proteins is assessed using Western blots. The samples used for western blots are untreated denatured samples (i.e., boiled in the presence of SDS, without a treatment with a reducing agent or a deglycosylation agent) are compared with (i) reduced samples, which are not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the chimeric proteins, the gels are run in triplicates and probed using an anti-TNFR2 antibody, an anti-IgG+IgM (H+L) antibody, or an anti-Clec7a/langerin/DC-SIGN/Dectin-2 antibody. The western blots are anticipated to show the efficient expression of the TNFR2-Fc-Clec7a, TNFR2-Fc-langerin, TNFR2-Fc-DC-SIGN, and TNFR2-Fc-Dectin-2 chimeric proteins.

### Example 9. Detection of Modified mRNA (mmRNA) Encoding the Chimeric Proteins or the Chimeric Proteins Themselves Following Transfection of the mmRNA in Cells

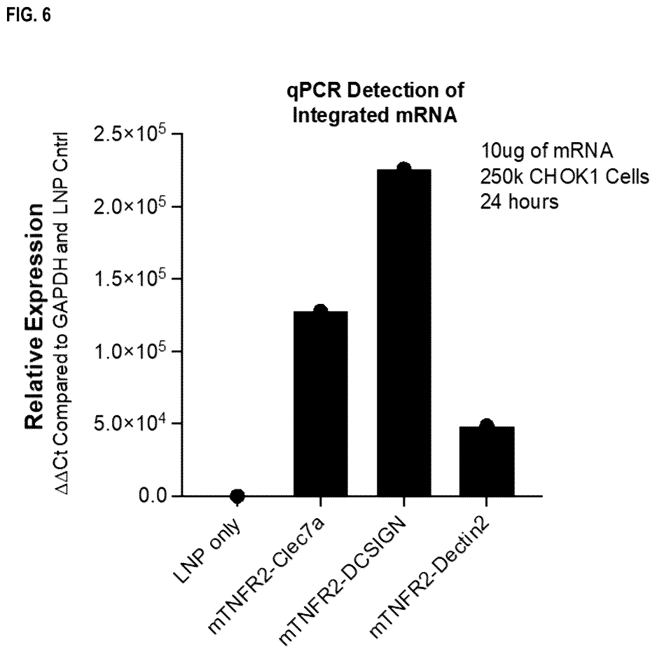

The modified mRNA (mmRNA) encoding the TNFR2-Fc-Clec7a, TNFR2-Fc-DC-SIGN, TNFR2-Fc-Dectin2 or TNFR2-Fc-Langerin chimeric proteins was complexed with Polyplus JetMessenger mRNA reagent to form lipid nanoparticles (LNPs). LNP lacking an mmRNA (LNP only) was also prepared for use as a negative control. LNP lacking an mmRNA (LNP only) was also prepared for use as a negative control. The LNP only or LNP containing 100 μg mmRNA encoding the TNFR2-Fc-Clec7a, TNFR2-Fc-DC-SIGN or TNFR2-Fc-Dectin2 chimeric proteins was transfected into 250,000 CHOK1 cells. After 24 hours, RNA was harvested from cell pellets, reverse transcribed, and amplified using qPCR. The differences in the cycle threshold (Ct) between primers amplifying mTNFR2 and the house-keeping control GAPDH were used to assess the relative expression of the mRNA constructs using the ΔΔCt method. As shown in FIG. 6, mRNA encoding the TNFR2-Fc-Clec7a, TNFR2-Fc-DC-SIGN or TNFR2-Fc-Dectin2 chimeric proteins could be detected after 24 hours. In contrast, the LNP only control showed only a background level of amplification.

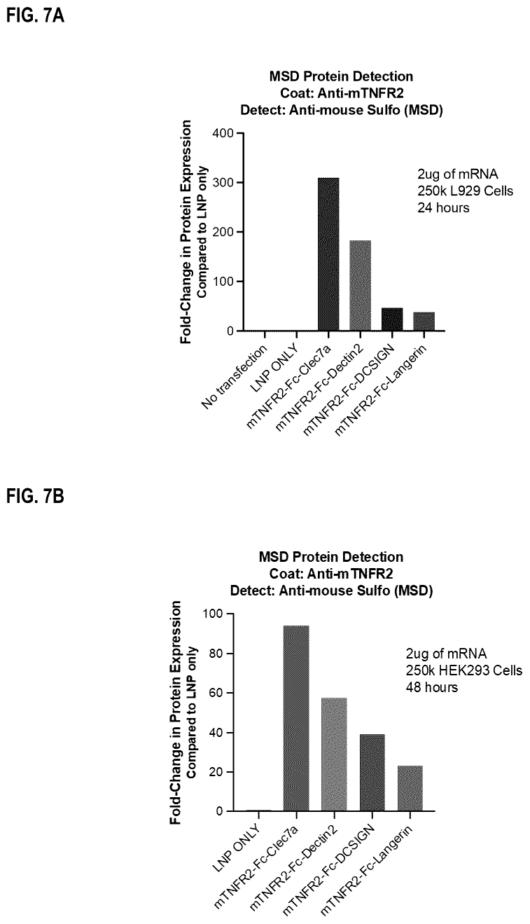

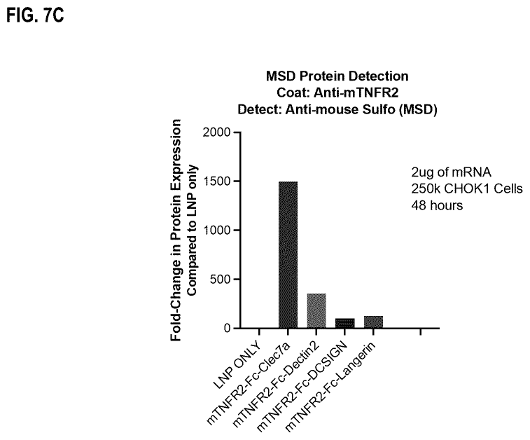

The LNP only or LNP containing 100 μg mmRNA encoding the TNFR2-Fc-Clec7a, TNFR2-Fc-DC-SIGN, TNFR2-Fc-Dectin2 or TNFR2-Fc-Langerin chimeric proteins was transfected into 250,000 L929 cells. After 24 hours, the culture supernatants were collected and the TNFR2-Fc-Clec7a, TNFR2-Fc-DC-SIGN, TNFR2-Fc-Dectin2 or TNFR2-Fc-Langerin chimeric proteins were detected using a Meso Scale Discovery (MSD) platform-based ELISA assay. Briefly, the anti-TNFR2 antibody was coated on a plate. The culture supernatants were added to the plate for capture by the plate-bound the anti-TNFR2 antibody of chimeric proteins secreted by L929 cells. The proteins captured by the plate-bound anti-TNFR2 antibody were detected using a SULFO-TAG conjugated anti-mouse antibody. The MSD signal was normalized with the signal obtained using the culture supernatant of the cells transfected with LNP only. As shown in FIG. 7A, the culture supernatants of the cells transfected with mmRNA encoding TNFR2-Fc-Clec7a, TNFR2-Fc-DC-SIGN, TNFR2-Fc-Dectin2, and TNFR2-Fc-Langerin chimeric proteins produced about 40 to about 300 times more MSD signal compared to that of LNP only-transfected cells. In contrast, the culture supernatants of untransfected cells or LNP-only transfected cells produced a background signal only (FIG. 7A). These results indicate, inter alia, that the mmRNA encoding the TNFR2-Fc-Clec7a, TNFR2-Fc-DC-SIGN, TNFR2-Fc-Dectin2, and TNFR2-Fc-Langerin chimeric proteins was efficiently translated in L929 cells to produce the chimeric proteins.

The LNP only or LNP containing 100 μg mmRNA encoding the TNFR2-Fc-Clec7a, TNFR2-Fc-DC-SIGN, TNFR2-Fc-Dectin2 or TNFR2-Fc-Langerin chimeric proteins was transfected into 250,000 HEK293 cells. After 48 hours, the culture supernatants were collected and the TNFR2-Fc-Clec7a, TNFR2-Fc-DC-SIGN, TNFR2-Fc-Dectin2 or TNFR2-Fc-Langerin chimeric proteins were detected using a Meso Scale Discovery (MSD) platform-based ELISA assay. Briefly, the anti-TNFR2 antibody was coated on a plate. The culture supernatants were added to the plate for capture by the plate-bound the anti-TNFR2 antibody of chimeric proteins secreted by HEK293 cells. The proteins captured by the plate-bound anti-TNFR2 antibody were detected using a SULFO-TAG conjugated anti-mouse antibody. The MSD signal was normalized with the signal obtained using the culture supernatant of the cells transfected with LNP only. As shown in FIG. 7B, the culture supernatants of the cells transfected with mmRNA encoding TNFR2-Fc-Clec7a, TNFR2-Fc-DC-SIGN, TNFR2-Fc-Dectin2, and TNFR2-Fc-Langerin chimeric proteins produced about 20 to about 100 times more MSD signal compared to that of LNP only-transfected cells. In contrast, the culture supernatants of untransfected cells or LNP-only transfected cells produced a background signal only (FIG. 7B). These results indicate, inter alia, that the mmRNA encoding the TNFR2-Fc-Clec7a, TNFR2-Fc-DC-SIGN, TNFR2-Fc-Dectin2, and TNFR2-Fc-Langerin chimeric proteins was efficiently translated in HEK293 cells to produce the chimeric proteins.

The LNP only or LNP containing 100 μg mmRNA encoding the TNFR2-Fc-Clec7a, TNFR2-Fc-DC-SIGN, TNFR2-Fc-Dectin2 or TNFR2-Fc-Langerin chimeric proteins was transfected into 250,000 CHOK 1 cells. After 48 hours, the culture supernatants were collected and the TNFR2-Fc-Clec7a, TNFR2-Fc-DC-SIGN, TNFR2-Fc-Dectin2 or TNFR2-Fc-Langerin chimeric proteins were detected using a Meso Scale Discovery (MSD) platform-based ELISA assay. Briefly, the anti-TNFR2 antibody was coated on a plate. The culture supernatants were added to the plate for capture by the plate-bound the anti-TNFR2 antibody of chimeric proteins secreted by CHOK1 cells. The proteins captured by the plate-bound anti-TNFR2 antibody were detected using a SULFO-TAG conjugated anti-mouse antibody. The MSD signal was normalized with the signal obtained using the culture supernatant of the cells transfected with LNP only. As shown in FIG. 7C, the culture supernatants of the cells transfected with mmRNA encoding TNFR2-Fc-Clec7a, TNFR2-Fc-DC-SIGN, TNFR2-Fc-Dectin2, and TNER2-Fc-Langerin chimeric proteins produced about 100 to about 1500 times more MSD signal compared to that of LNP only-transfected cells. In contrast, the culture supernatants of untransfected cells or LNP-only transfected cells produced a background signal only (FIG. 7C). These results indicate, inter alia, that the mmRNA encoding the TNFR2-Fc-Clec7a, TNFR2-Fc-DC-SIGN, TNFR2-Fc-Dectin2, and TNFR2-Fc-Langerin chimeric proteins was efficiently translated in CHOK1 cells to produce the chimeric proteins.

Collectively, these results demonstrate, inter alia, that mmRNA encoding TNFR2-Fc-Clec7a, TNFR2-Fc-DC-SIGN, TNFR2-Fc-Dectin2, and TNFR2-Fc-Langerin chimeric proteins was stable and showed a sustained expression of TNFR2-Fc-Clec7a, TNFR2-Fc-DC-SIGN, TNFR2-Fc-Dectin2, and TNFR2-Fc-Langerin chimeric proteins in cells harboring the mmRNA.

### Example 10. The Chimeric Proteins Disclosed Herein Block TNFα or Zymosan-Mediated NFκB Induction in Reporter Cells

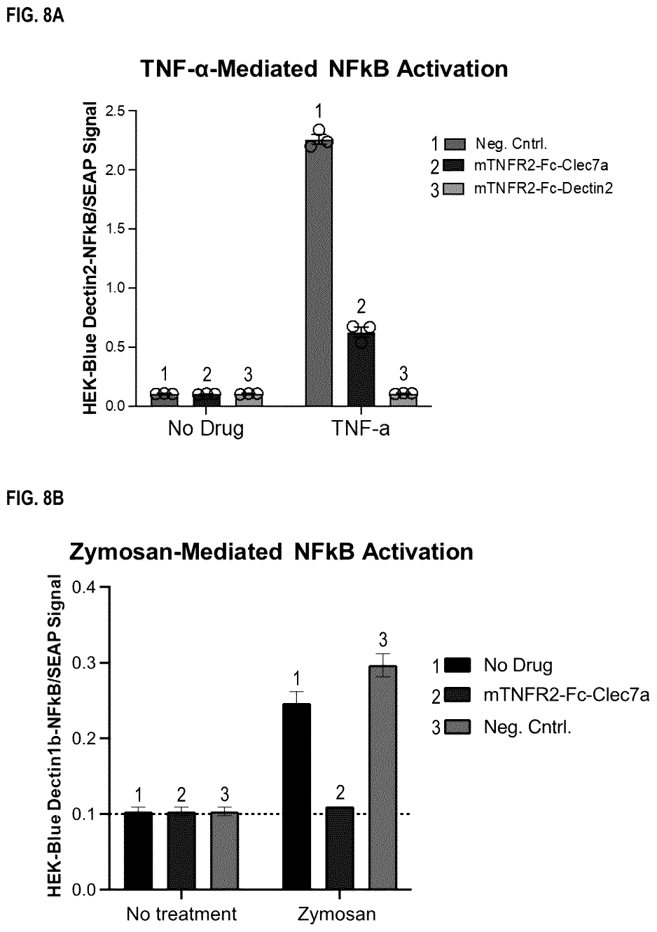

The effect of the chimeric proteins disclosed herein on signal transduction induced by TNFα was performed using the HEK-Blue® Dectin2 cells that carry a secreted alkaline phosphatase (SEAP) reporter gene under the control of a minimal promoter fused to five NFκB and AP-1 binding sites. Briefly, the HEK-Blue® Dectin2 cells that carry the SEAP reporter gene were incubated with the TNFR2-Fc-Clec7a or TNFR2-Fc-Dectin2 chimeric proteins or an irrelevant protein that was used as a negative control in the presence or absence of TNFα. SEAP signal was measured after the incubation. As shown in FIG. 8A, the TNFR2-Fc-Clec7a or TNFR2-Fc-Dectin2 chimeric proteins or the irrelevant protein showed a background level of SEAP activity (reading of about 0.1 in each case). Addition of TNFα in the presence of the irrelevant protein increased the SEAP activity by more than 20 fold (FIG. 8A). Interestingly, compared to the combination of TNFα and the irrelevant protein, the TNFR2-Fc-Clec7a and TNER2-Fc-Dectin2 chimeric proteins, produced an about 4 and more than 20-fold reduction in SEAP activity (FIG. 8A).

These results indicate, inter alia, that the TNFR2-based chimeric proteins disclosed herein sequester TNFα and thereby reduce TNFα-induced NFκB induction and downstream signaling.

The effect of the chimeric proteins disclosed herein on signal transduction induced by zymosan was performed using the HEK-Blue® Dectin1b cells that carry a secreted alkaline phosphatase (SEAP) reporter gene under the control of a minimal promoter fused to five NFκB and AP-1 binding sites. Briefly, the HEK-Blue® Dectin1b cells that carry the SEAP reporter gene were incubated with buffer only, the TNFR2-Fc-Clec7a chimeric protein or an irrelevant protein that was used as a negative control in the presence or absence of zymosan. SEAP signal was measured after the incubation. As shown in FIG. 8B, incubation of the HEK-Blue® Dectin1b cells that carry the SEAP reporter gene with the buffer only, the TNFR2-Fc-Clec7a chimeric protein or the irrelevant protein showed a background level of SEAP activity (reading of about 0.1 in each case). Addition of zymosan in the presence of buffer only increased the SEAP activity by about 2-3 fold (FIG. 8B). Interestingly, compared to the zymosan alone treatment (with buffer only), the co-treatment with the TNFR2-Fc-Clec7a chimeric protein, produced an about 2-3 fold reduction in SEAP activity (FIG. 8B), bringing the activity back to background levels. Coincubation with the irrelevant protein showed no such reduction in SEAP activity (FIG. 8B).

These results indicate, inter alia, that the TNFR2-Fc-Clec7a chimeric protein sequesters zymosan and thereby reduce zymosan-induced NFκB induction and downstream signaling. Collectively, these results indicate, inter alia, that the TNFR2-based chimeric proteins disclosed herein sequester TNFα and C-type lectin ligands, and thereby reduce NFκB induction and downstream signaling.

### Example 11. The Modified mRNA (mmRNA) Encoding the Chimeric Proteins Disclosed Herein or the Purified Chimeric Proteins Block TNFα-Induced Apoptosis of Sensitive Cells

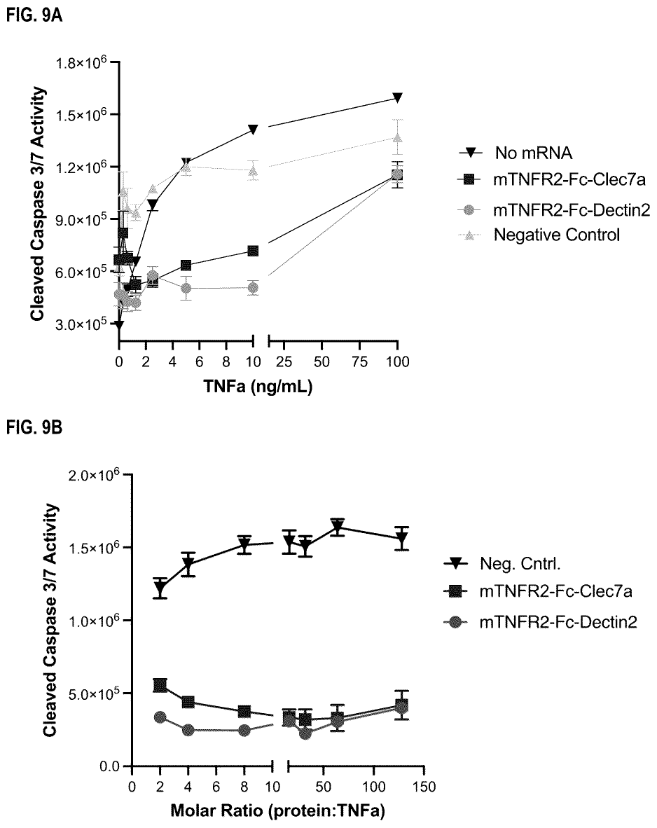

The effect of the purified TNFR2-Fc-Clec7a and TNFR2-Fc-Dectin2 chimeric proteins on apoptosis induced by TNFα in L929 fibroblast cells was analyzed. Briefly, L929 fibroblast cells were co-incubated with the purified TNFR2-Fc-Clec7a and TNFR2-Fc-Dectin2 chimeric proteins or an irrelevant protein, which was used as a negative control, in the presence of the increasing molar ratios of TNFα. After the incubation, the extent of apoptosis, as measured by cleaved caspase 3/7 activity, was plotted as a function of molar ratio of the chimeric protein/irrelevant protein to TNFα. As shown in FIG. 9B, the L929 fibroblast cells treated with irrelevant protein and TNFα exhibited a dose-dependent apoptosis with increasing concentration of TNFα. In contrast, as shown in FIG. 9B, the L929 fibroblast cells treated with the TNFR2-Fc-Clec7a and TNFR2-Fc-Dectin2 chimeric proteins exhibited a protection from the TNFα-mediated apoptosis.

These results demonstrate, inter alia, that the TNFR2-based chimeric proteins disclosed herein sequester TNFα and thereby protect cells from TNFα-mediated apoptosis.

The effect of the modified mRNA (mmRNA) encoding the TNFR2-Fc-Clec7a and TNFR2-Fc-Dectin2 chimeric proteins on apoptosis induced by TNFα in L929 fibroblast cells was analyzed. Briefly, the modified mRNA (mmRNA) encoding the TNFR2-Fc-Clec7a and TNFR2-Fc-Dectin2 chimeric proteins was complexed with Polyplus JetMessenger mRNA reagent to form lipid nanoparticles (LNPs). Empty LNP lacking an mmRNA (no mRNA) or LNPs harboring mmRNA encoding an irrelevant protein, which was used as a negative control, were also prepared for use as a negative control. L929 fibroblast cells were transfected using the LNP and then incubated with increasing amounts of TNFα. After the incubation, the extent of apoptosis, as measured by cleaved caspase 3/7 activity, was plotted as a function of amount of TNFα. As shown in FIG. 9A, the L929 fibroblast cells transfected with empty LNP (no mRNA) exhibited a dose-dependent apoptosis with increasing concentration of TNFα. The L929 fibroblast cells transfected with LNP containing mRNA encoding the irrelevant protein (negative control) also exhibited a dose-dependent apoptosis with increasing concentration of TNFα (FIG. 9A). In contrast, as shown in FIG. 9A, the L929 fibroblast cells transfected with LNP containing mRNA encoding the encoding the TNFR2-Fc-Clec7a and TNFR2-Fc-Dectin2 chimeric proteins exhibited a protection from the TNFα-mediated apoptosis.

These results demonstrate, inter alia, that cells transfected with the mmRNA encoding TNFR2-based chimeric proteins disclosed herein produce sufficient levels of the functional TNFR2-based chimeric proteins to effectively sequester TNFα and to protect the cells themselves from TNFα-mediated apoptosis.

### Example 12. In Vivo Efficacy of the Chimeric Proteins Disclosed Herein in Mouse Model of Colitis

This Dextran sulfate sodium (DSS)-induced colitis model was used to evaluate the in vivo efficacy of the mmRNA encoding the chimeric proteins disclosed herein. DSS is a polysaccharide, which when administered in the drinking water of mice, induces colitis, associated with weight loss, increased TNFα production, and the stimulation of innate and adaptive inflammatory responses.

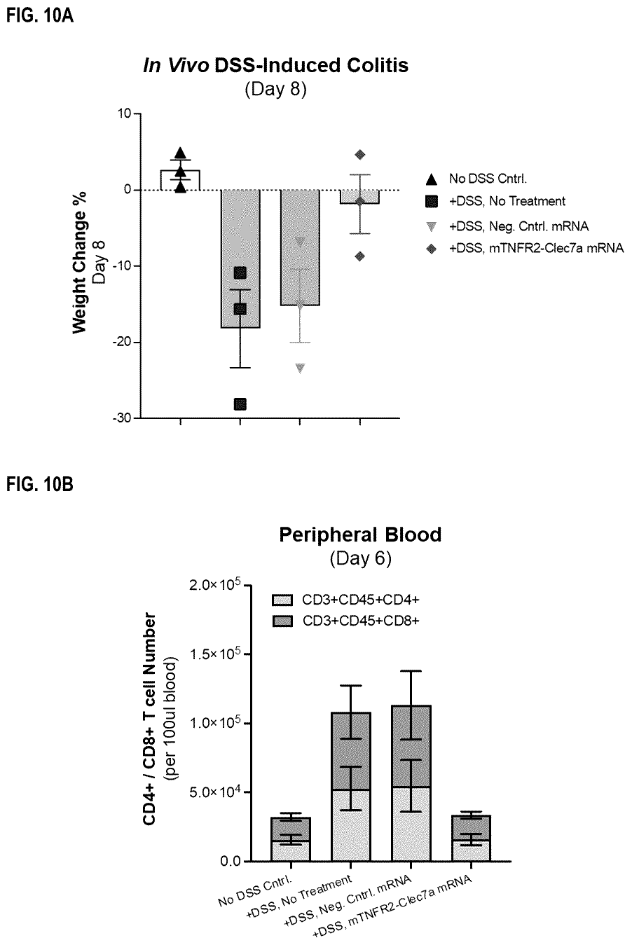

Briefly, the modified mRNA (mmRNA) encoding the TNFR2-Fc-Clec7a chimeric protein or an irrelevant protein, which was used as a negative control, were complexed with Polyplus JetMessenger mRNA reagent to form lipid nanoparticles (LNPs). Mice were weighed and sorted into the following treatment groups: (1) No DSS, (2) DSS+no LNP (no treatment), (3) DSS+LNP containing the mmRNA encoding the irrelevant protein, and (4) DSS+LNP containing the mmRNA encoding the TNFR2-Fc-Clec7a chimeric protein. On day 0, experimental treatment group animals were administered 3% dextran sodium sulfate (DSS) in their drinking water, ad libitum for 8 days. Control animals (Group 1, No DSS control) were administered unmodified drinking water. On day 1, animals of Group 3 were administered single IV infusion of LNP containing the 0.5 mg/kg mmRNA encoding the irrelevant protein, and animals of Group 3 were administered single IV infusion of LNP containing the 0.5 mg/kg mmRNA encoding the TNFR2-Fc-Clec7a chimeric protein. The animals were monitored daily for signs of distress and weighed. FIG. 10A is a bar graph showing the change in body weight on day 8. As shown in FIG. 10A, compared to the no DSS control, the DSS-treated mice that received no further treatment showed a reduction in weight by about 15% (compare first bar and second bar in FIG. 10A). The mice that received LNP containing the 0.5 mg/kg mmRNA encoding the irrelevant protein also exhibited a reduction in weight by about 15%, similar to that in the DSS-exposed, untreated mice (compare second bar and third bar in FIG. 10A). The mice that received LNP containing the 0.5 mg/kg mmRNA encoding the TNFR2-Fc-Clec7a chimeric protein exhibited minimal weight loss, compared to the DSS-exposed, untreated mice (compare second bar and fourth bar in FIG. 10A). These results indicate, inter alia, that a single injection with mmRNA encoding the chimeric proteins disclosed herein reverse the colitis induced by DSS.

On Day 6, peripheral blood was extracted from the animals, stained with a panel of antibodies that included anti-CD3, anti-CD4, anti-CD8, and anti-CD45 antibodies and analyzed by flow cytometry. As shown in FIG. 10B, compared to the mice that did not receive DSS, the mice that received DSS but no further treatment exhibited increased CD3+CD45+CD4+ and CD3+CD45+CD8+ cells out of total CD4+/CD8+ cells, indicating an increase in inflammation (FIG. 10B). The mice that received LNP containing the 0.5 mg/kg mmRNA encoding the irrelevant protein also exhibited an increased CD3+CD45+CD4+ and CD3+CD45+CD8+ cells out of total CD4+/CD8+ cells, similar to that in the DSS-exposed, untreated mice (compare second bar and third bar in FIG. 10B). The mice that received LNP containing the 0.5 mg/kg mmRNA encoding the TNFR2-Fc-Clec7a chimeric protein exhibited a complete reversal of the increase in CD3+CD45+CD4+ and CD3+CD45+CD8+ cells out of total CD4+/CD8+ cells (compare second bar and fourth bar in FIG. 10B), resulting the restoration of the levels of CD3+CD45+CD4+ and CD3+CD45+CD8+ cells out of total CD4+/CD8+ cells similar to those in the mice that did not receive DSS (compare first bar and fourth bar in FIG. 10A). These results indicate, inter alia, that a single injection with mmRNA encoding the chimeric proteins disclosed herein the mmRNA provides sustained biological effect after administration and reverse the cellular changes brought about by colitis. Accordingly, inter alia, the mmRNA encoding the chimeric proteins disclosed herein may be used in therapeutic methods where suppressing inflammation is beneficial.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present technology is not entitled to antedate such publication by virtue of prior disclosure.

As used herein, all headings are simply for organization and are not intended to limit the disclosure in any manner. The content of any individual section may be equally applicable to all sections.

### EQUIVALENTS

While the disclosure has been disclosed in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A chimeric protein having a general structure of: 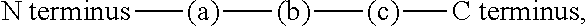
wherein:
(a) is a first domain comprising a portion of TNF receptor (TNFR2) that is capable of binding TNFα and/or capable of oligomerizing with a cellular TNF receptor;
(b) is a linker adjoining the first domain and a second domain, optionally comprising a hinge-C_{H2}-C_{H3} Fc domain, and
(c) is a second domain comprising a portion of a C-type lectin receptor (CLR) capable of binding a ligand.

2. The chimeric protein of claim 1, wherein the portion of TNFR2 comprises the extracellular domain of TNFR2, or a fragment thereof.

3. The chimeric protein of claim 1 or claim 2, wherein the portion of TNFR2 comprises an amino acid sequence that is at least about 90%, or at least about 95%, or at least about 97%, or at least about 98%, or at least about 99% identical to the amino acid sequence of SEQ ID NO: 57.

4. The chimeric protein of any one of claims 1 to 3, wherein the CLR is selected from C-type lectin domain containing 7A(Clec7A), langerin, dendritic cell-specific intercellular adhesion molecule-3-grabbing non-integrin (DC-SIGN), and dendritic cell-associated C-type lectin-2 (Dectin-2).

5. The chimeric protein of any one of claims 1 to 4, wherein the second domain comprises a portion of Clec7a.

6. The chimeric protein of claim 5, wherein the portion of Clec7a comprises the extracellular domain of Clec7a, or a fragment thereof capable of binding a beta-1,3-linked and/or beta-1,6-linked glucan.

7. The chimeric protein of claim 5 or claim 6, wherein the portion of Clec7a comprises an amino acid sequence that is at least about 90%, or at least about 95%, or at least about 97%, or at least about 98%, or at least about 99% identical to the amino acid sequence of SEQ ID NO: 58 or SEQ ID NO: 59.

8. The chimeric protein of any one of claims 5 to 7, wherein the chimeric protein comprises:
an extracellular domain of TNFR2 comprising an amino acid sequence that is at least about 90%, or at least about 95%, or at least about 97%, or at least about 98%, or at least about 99% identical to the amino acid sequence of SEQ ID NO: 57;
a portion of Clec7a comprising an amino acid sequence that is at least about 90%, or at least about 95%, or at least about 97%, or at least about 98%, or at least about 99% identical to the amino acid sequence of SEQ ID NO: 58 or SEQ ID NO: 59; and
a linker adjoining the extracellular domains.

9. The chimeric protein of any one of claims 1 to 4, wherein the second domain comprises a portion of langerin.

10. The chimeric protein of claim 9, wherein the portion of langerin comprises the extracellular domain of langerin, or a fragment thereof capable of binding a sulfated glycan, a mannosylated glycan, a keratan sulfate (KS) and/or a beta-glucan.

11. The chimeric protein of claim 9 or claim 10, wherein the portion of langerin comprises an amino acid sequence that is at least about 90%, or at least about 95%, or at least about 97%, or at least about 98%, or at least about 99% identical to the amino acid sequence of SEQ ID NO: 60 or SEQ ID NO: 61.

12. The chimeric protein of any one of claims 9 to 11, wherein the chimeric protein comprises:
an extracellular domain of TNFR2 comprising an amino acid sequence that is at least about 90%, or at least about 95%, or at least about 97%, or at least about 98%, or at least about 99% identical to the amino acid sequence of SEQ ID NO: 57;
a portion of langerin comprising an amino acid sequence that is at least about 90%, or at least about 95%, or at least about 97%, or at least about 98%, or at least about 99% identical to the amino acid sequence of SEQ ID NO: 60 or SEQ ID NO: 61; and
a linker adjoining the extracellular domains.

13. The chimeric protein of any one of claims 1 to 4, wherein the second domain comprises a portion of DC-SIGN.

14. The chimeric protein of claim 13, wherein the portion of DC-SIGN comprises the extracellular domain of DC-SIGN, or a fragment thereof capable of binding Intercellular Adhesion Molecule 2 (ICAM2) and/or Intercellular Adhesion Molecule 3 (ICAM3).

15. The chimeric protein of claim 13 or claim 14, wherein the portion of DC-SIGN comprises an amino acid sequence that is at least about 90%, or at least about 95%, or at least about 97%, or at least about 98%, or at least about 99% identical to the amino acid sequence of SEQ ID NO: 62 or SEQ ID NO: 63.

16. The chimeric protein of any one of claims 13 to 15, wherein the chimeric protein comprises:
an extracellular domain of TNFR2 comprising an amino acid sequence that is at least about 90%, or at least about 95%, or at least about 97%, or at least about 98%, or at least about 99% identical to the amino acid sequence of SEQ ID NO: 57;
a portion of DC-SIGN comprising an amino acid sequence that is at least about 90%, or at least about 95%, or at least about 97%, or at least about 98%, or at least about 99% identical to the amino acid sequence of SEQ ID NO: 62 or SEQ ID NO: 63; and
a linker adjoining the extracellular domains.

17. The chimeric protein of any one of claims 1 to 4, wherein the second domain comprises a portion of Dectin-2.

18. The chimeric protein of claim 17, wherein the portion of Dectin-2 comprises the extracellular domain of Dectin-2, or a fragment thereof capable of binding an alpha-mannan.

19. The chimeric protein of claim 17 or claim 18, wherein the portion of Dectin-2 comprises an amino acid sequence that is at least about 90%, or at least about 95%, or at least about 97%, or at least about 98%, or at least about 99% identical to the amino acid sequence of SEQ ID NO: 64 or SEQ ID NO: 65.

20. The chimeric protein of any one of claims 17 to 19, wherein the chimeric protein comprises:
an extracellular domain of TNFR2 comprising an amino acid sequence that is at least about 90%, or at least about 95%, or at least about 97%, or at least about 98%, or at least about 99% identical to the amino acid sequence of SEQ ID NO: 57;
a portion of Dectin-2 comprising an amino acid sequence that is at least about 90%, or at least about 95%, or at least about 97%, or at least about 98%, or at least about 99% identical to the amino acid sequence of SEQ ID NO: 64 or SEQ ID NO: 65; and
a linker adjoining the extracellular domains.

21. The chimeric protein of any one of claims 1 to 20, wherein the hinge-C_{H2}-C_{H3} Fc domain is derived from lgG1 or lgG4, e.g., human lgG1 or lgG4.

22. The chimeric protein of claim 21, wherein the hinge-C_{H2}-C_{H3} Fc domain comprises an amino acid sequence that is at least about 95% identical to the amino acid sequence of SEQ ID NO: 1, SEQ ID NO: 2, SEQ ID NO: 3, or SED ID NO: 73.

23. The chimeric protein of claim 21 or claim 22, wherein the linker further comprises the linker comprises one or more joining linkers, such joining linkers independently selected from SEQ ID NOs: 4 to 50.

24. The chimeric protein of any one of claims 21 to 23, wherein the linker comprises two or more joining linkers each joining linker independently selected from SEQ ID NOs: 4 to 50; wherein one joining linker is N terminal to the hinge-C_{H2}-C_{H3}-Fc domain and another joining linker is C terminal to the hinge-C_{H2}-C_{H3}-Fc domain.

25. An isolated polynucleotide encoding the chimeric protein of any one of claims 1 to 24.

26. The isolated polynucleotide of claim 25, wherein the polynucleotide is or comprises an mRNA or a modified mRNA (mmRNA).

27. The isolated polynucleotide of claim 25 or claim 26, wherein the polynucleotide is or comprises an mmRNA.

28. The isolated polynucleotide of claim 27, wherein the mmRNA comprises one or more nucleoside modifications.

29. The isolated polynucleotide of claim 28, wherein the nucleoside modifications are selected from pyridin-4-one ribonucleoside, 5-aza-uridine, 2-thio-5-aza-uridine, 2-thiouridine, pseudouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6, N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2, N2-dimethyl-6-thio-guanosine, and combinations thereof.

30. The isolated polynucleotide of any one of claims 27 to 29, wherein the mmRNA further comprises a 5′-cap and/or a poly A tail.

31. The isolated polynucleotide of claim 25, wherein the polynucleotide is DNA.

32. The isolated polynucleotide of claim 31, wherein the polynucleotide comprises a skin-specific control element.

33. The isolated polynucleotide of claim 32, wherein the skin-specific control element is a skin-specific promoter selected from a keratin 5 (K5) promoter, a keratin 6 (K6) promoter, a keratin 14 (K14) promoter, a keratin 16 (K16) promoter, an alpha-1 (I) collagen promoter, a filaggrin promoter, a loricrin promoter, an involucrin promoter, a tyrosinase promoter, and an V integrin promoter.

34. The isolated polynucleotide of claim 25 or claim 26, wherein the polynucleotide is or comprises an mRNA.

35. A vector comprising the polynucleotide of any one of claims 31 to 34.

36. A host cell comprising the polynucleotide of any one of claims 25 to 34.

37. A host cell comprising the vector of claim 35.

38. A pharmaceutical composition comprising a pharmaceutically acceptable excipient or carrier, and the chimeric protein of any one of claims 1 to 24, the isolated polynucleotide of any one of claims 25 to 34, the mmRNA of any one of claims 26 to 30, the vector of claim 35, or the host cell of claim 37.

39. The pharmaceutical composition of claim 38, wherein the pharmaceutical composition comprises the mmRNA of any one of claims 26 to 30.

40. The pharmaceutical composition of claim 38 or claim 39, wherein the carrier is a lipidoid, a liposome, a lipoplex, a lipid nanoparticle, a polymeric nanoparticle, a peptide, a protein, a cell, a nanoparticle mimic, a nanotube, or a conjugate.

41. The pharmaceutical composition of any one of claims 38 to 40, wherein the pharmaceutical composition is formulated as a lipid nanoparticle (LNP), a lipoplex, or a liposome.

42. The pharmaceutical composition of claim 41, wherein the pharmaceutical composition is formulated as a lipid nanoparticle (LNP).

43. The pharmaceutical composition of claim 42, wherein the lipid nanoparticles comprise lipids selected from an ionizable lipid (e.g. an ionizable cationic lipid selected from DLin-DMA, DLin-K-DMA, DLin-KC2-DMA, DLin-MC3-DMA, 98N12-5, and C12-200); a structural lipid (e.g. distearoylphosphatidylcholine (DSPC));
cholesterol, and a polyethyleneglycol (PEG)-lipid (e.g. a PEG-diacylglycerol (DAG), a PEG-dialkyloxypropyl (DAA), a PEG-phospholipid, a PEG-ceramide (Cer), or a mixture thereof, or a PEG-dilauryloxypropyl (C12, a PEG-dimyristyloxypropyl (C14), a PEG-dipalmityloxypropyl (C16), or a PEG-distearyloxypropyl (C18)); 1,2-dioleoyl-3-trimethylammoniumpropane (DOTAP); dioleoylphosphatidylethanolamine (DOPE).

44. The pharmaceutical composition of claim 42 or claim 43, wherein the lipid nanoparticles comprise (a) a cationic lipid comprising from 50 mol % to 85 mol % of the total lipid present in the particle; (b) a non-cationic lipid comprising from 13 mol % to 49.5 mol % of the total lipid present in the particle; and (c) a conjugated lipid that inhibits aggregation of particles comprising from 0.5 mol % to 2 mol % of the total lipid present in the particle.

45. The pharmaceutical composition of any one of claims 42 to 44, wherein the lipid nanoparticles comprise a lipid selected from SM-102, DLin-DMA, DLin-K-DMA, DLin-KC2-DMA, DLin-MC3-DMA, 98N12-5, and C12-200; a cholesterol; and a PEG-lipid.

46. The pharmaceutical composition of any one of claims 38 to 45, wherein the pharmaceutical composition is formulated for parenteral administration.

47. The pharmaceutical composition of any one of claims 38 to 45, wherein the pharmaceutical composition is formulated for topical, dermal, intradermal, intramuscular, intraperitoneal, intraarticular, intravenous, subcutaneous, intraarterial or transdermal administration.

48. The pharmaceutical composition of any one of claims 38 to 45, wherein the pharmaceutical composition is formulated for topical administration.

49. A method of treating or preventing inflammation of the integumentary system, the method comprising administering to a subject the chimeric protein of any one of claims 1 to 24, the isolated polynucleotide of any one of claims 25 to 34, the mmRNA of any one of claims 26 to 30, the vector of claim 35, or the host cell of claim 37.

50. A method of treating or preventing inflammation of the integumentary system, the method comprising administering to a subject the pharmaceutical composition of any one of claims 38 to 48.

51. The method of claim 49 or claim 50, wherein the inflammation is caused by or associated with a disease or disorder of the integumentary system.

52. The method of any one of claims 49 to 51, wherein the inflammation is caused by or associated with a disease or disorder of the skin.

53. The method of claim 52, wherein the disease or disorder of the skin is psoriasis, pemphigus vulgaris, scleroderma, atopic dermatitis, sarcoidosis, erythema nodosum, hidradenitis suppurativa, lichen planus, Sweet's syndrome, vitiligo, chronic paronychia, eczema, seborrheic dermatitis, and/or hives.

54. The method of claim 52 or claim 53, wherein the disease or disorder of the skin is a psoriasis.

55. The method of claim 54, wherein the psoriasis is plaque psoriasis.

56. The method of claim 54, wherein the psoriasis is psoriatic arthritis.

57. The method of any one of claims 49 to 56, wherein the inflammation is mediated by macrophages and/or dendritic cells.

58. The method of any one of claims 49 to 57, wherein the treatment reduces:
the levels of infiltration of T cells, neutrophils, dendritic cells, macrophages, and/or NK cells in the inflamed tissue compared to the levels of infiltration of T cells, neutrophils, dendritic cells, macrophages, and/or NK cells prior to the treatment; and/or
the levels of TNFα, IL-17 and/or IL-23 in the inflamed tissue compared to the levels of TNFα, IL-17 and/or IL-23 prior to the treatment.

59. The method of any one of claims 54 to 58, wherein the treatment reduces redness, thickening, and flaking of the skin compared to the reduces redness, thickening, and flaking of the skin prior to the treatment.

60. The method of any one of claims 54 to 59, further comprising administering to the subject an anti-inflammatory drug.

61. The method of claim 60, wherein the anti-inflammatory drug is a non-steroidal anti-inflammatory or a corticosteroid.

62. The method of claim 60 or claim 61, wherein the pharmaceutical composition and the anti-inflammatory drug are provided concurrently.

63. The method of claim 62, wherein the pharmaceutical composition and the anti-inflammatory drug are provided as two distinct pharmaceutical compositions.

64. The method of claim 62, wherein the pharmaceutical composition and the anti-inflammatory drug are provided as a single pharmaceutical composition.

65. The method of claim 60 or claim 61, wherein the pharmaceutical composition is provided after the anti-inflammatory drug is provided.

66. The method of claim 60 or claim 61, wherein the pharmaceutical composition is provided before the anti-inflammatory drug is provided.

67. The method of any one of claims 61 to 66, wherein the anti-inflammatory drug is a non-steroidal anti-inflammatory agent selected from acetyl salicylic acid (aspirin), benzyl-2,5-diacetoxybenzoic acid, celecoxib, diclofenac, etodolac, etofenamate, fulindac, glycol salicylate, ibuprofen, indomethacin, ketoprofen, methyl salicylate, nabumetone, naproxen, oxaprozin, phenylbutazone, piroxicam, salicylic acid, salicylmides, and esomeprazole, or a combination of any two or more thereof.

68. The method of any one of claims 61 to 66, wherein the anti-inflammatory drug is a corticosteroid selected from alpha-methyl dexamethasone, amcinafel, amcinafide, beclomethasone dipropionate, beclomethasone dipropionate., betamethasone and the balance of its esters, betamethasone benzoate, betamethasone dipropionate, betamethasone valerate, beta-methyl betamethasone, bethamethasone, chloroprednisone, clescinolone, clobetasol valerate, clocortelone, cortisone, cortodoxone, desonide, desoxymethasone, dexamethasone, dichlorisone, diflorasone diacetate, diflucortolone valerate, difluorosone diacetate, difluprednate, fluadrenolone, flucetonide, fluclorolone acetonide, flucloronide, flucortine butylester, fludrocortisone, flumethasone pivalate, flunisolide, fluocinonide, fluocortolone, fluoromethalone, fluosinolone acetonide, fluperolone, fluprednidene (fluprednylidene) acetate, fluprednisolone, fluradrenolone acetonide, flurandrenolone, halcinonide, hydrocortisone, hydrocortisone acetate, hydrocortisone butyrate, hydroxyltriamcinolone, medrysone, meprednisone, methylprednisolone, paramethasone, prednisolone, prednisone, triamcinolone, and triamcinolone acetonide.

69. The method of any one of claims 54 to 59, further comprising administering to the subject an immunosuppressive agent.

70. The method of claim 69, wherein the pharmaceutical composition and the immunosuppressive agent are provided concurrently.

71. The method of claim 69, wherein the pharmaceutical composition and the immunosuppressive agent are provided as two distinct pharmaceutical compositions.

72. The method of claim 69, wherein the pharmaceutical composition and the immunosuppressive agent are provided as a single pharmaceutical composition.

73. The method of claim 69, wherein the pharmaceutical composition is provided after the immunosuppressive agent is provided.

74. The method of claim 69, wherein the pharmaceutical composition is provided before the immunosuppressive agent is provided.

75. The method of any one of claims 69 to 74, wherein the immunosuppressive agent is selected from an antibody (e.g., basiliximab, daclizumab, and muromonab), an anti-immunophilin (e.g., cyclosporine, tacrolimus, and sirolimus), an antimetabolite (e.g., azathioprine and methotrexate), a cytostatic (such as alkylating agents), a cytotoxic antibiotic, an inteferon, a mycophenolate, an opioid, a small biological agent (e.g., fingolimod and myriocin), and a TNF binding protein.

76. The method of any one of claims 54 to 59, further comprising administering to the subject an anti-inflammatory drug and an immunosuppressive agent.

77. The method of any one of claims 54 to 59, further comprising administering to the subject a second pharmaceutical composition comprising an IL-12/IL-23 inhibitor and/or an IL-17 inhibitor.

78. The method of claim 77, wherein the pharmaceutical composition and the immunosuppressive agent are provided concurrently.

79. The method of claim 77, wherein the pharmaceutical composition and the immunosuppressive agent are provided as two distinct pharmaceutical compositions.

80. The method of claim 77, wherein the pharmaceutical composition and the immunosuppressive agent are provided as a single pharmaceutical composition.

81. The method of claim 77, wherein the pharmaceutical composition is provided after the immunosuppressive agent is provided.

82. The method of claim 77, wherein the pharmaceutical composition is provided before the immunosuppressive agent is provided.

83. The method of any one of claims 69 to 74, wherein the IL-17 inhibitor is selected from secukinumab, ixekizumab, bimekizumab, and brodalumab.

84. The method of any one of claims 69 to 74, wherein the IL12/IL-23 inhibitor is selected from utsekinumab, risankizumab, guselkumab, and tildrakizumab.

85. The pharmaceutical composition of any one of claims 38 to 48 for use in treating or preventing an inflammation.

86. The chimeric protein of any one of claims 1 to 24, the isolated polynucleotide of any one of claims 25 to 34, the mmRNA of any one of claims 26 to 30, the vector of claim 35, or the host cell of claim 37 for use in treating or preventing an inflammation.
